(19) United States
(12) Patent Application Publication
(10) Pub. No.: US 2007/0265281 A1
(43) Pub. Date: Nov. 15, 2007

(54) Organic Compounds and Their Uses

(76) Inventors: Sylvain Cottens, Witterswil (CH); Claus Ehrhardt, Lorrach (DE); Jiping Fu, Arlington, MA (US); David Thomas Parker, Lexington, MA (US); Michael Patane, Reading, MA (US); Branko Radetich, Boston, MA (US); Prakash Raman, Acton, MA (US); Stefan Andreas Randl, Basel (CH); Pascal Rigollier, Mulhouse (FR); Mohindra Seepersaud, Belmont, MA (US); Oliver Simic, Basel (CH); Dongpeng Wan, North Quincy, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/697,901

(22) Filed: Apr. 9, 2007

Related U.S. Application Data
(60) Provisional application No. 60/791,578, filed on Apr. 11, 2006.

(51) Int. Cl. A61K 31/4965; A61K 31/16; A61K 31/18; C07C 233/00; C07D 209/04; C07D 241/02; C07C 303/00; A61P 31/00; A61K 31/405

(52) U.S. Cl. 514/255.06; 514/415; 514/602; 514/616; 544/406; 548/491; 564/153; 564/80

(57) ABSTRACT

The present application describes organic compounds that are useful for the treatment, prevention and/or amelioration of human diseases.

### BACKGROUND

Hepatitis C virus (HCV) is a (+)-sense single-stranded RNA virus that has been implicated as the major causative agent in non-A, non-B hepatitis (NANBH), particularly in blood-associated NANBH (BB-NANBH). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

Recently, an HCV protease necessary for polypeptide processing and viral replication has been identified, cloned and expressed. (See, e.g., U.S. Pat. No. 5,712,145). This approximately 3000 amino acid polyprotein contains, from the amino terminus to the carboxy terminus, a nucleocapsid protein (C), envelope proteins (E1 and E2) and several non-structural proteins (NS1, 2, 3, 4a, 5a and 5b). NS3 is an approximately 68 kda protein, encoded by approximately 1893 nucleotides of the HCV genome, and has two distinct domains: (a) a serine protease domain consisting of approximately 200 of the N-terminal amino acids; and (b) an RNA-dependent ATPase domain at the C-terminus of the protein. The NS3 protease is considered a member of the chymotrypsin family because of similarities in protein sequence, overall three-dimensional structure and mechanism of catalysis. The HCV NS3 serine protease is responsible for proteolysis of the polypeptide (polyprotein) at the NS3/NS4a, NS4a/NS4b, NS4b/NS5a and NS5a/NS5b junctions and is thus responsible for generating four viral proteins during viral replication. This has made the HCV NS3 serine protease an attractive target for antiviral chemotherapy.

It has been determined that the NS4a protein, an approximately 6 kda polypeptide, is a co-factor for the serine protease activity of NS3. Autocleavage of the NS3/NS4a junction by the NS3/NS4a serine protease occurs intramolecularly (i.e., cis) while the other cleavage sites are processed intermolecularly (i.e., trans).

HCV has been implicated in cirrhosis of the liver and in induction of hepatocellular carcinoma. The prognosis for patients suffering from HCV infection is currently poor. HCV infection is more difficult to treat than other forms of hepatitis due to the lack of immunity or remission associated with HCV infection. Current data indicates a less than 50% survival rate at four years post cirrhosis diagnosis. Patients diagnosed with localized resectable hepatocellular carcinoma have a five-year survival rate of 10-30%, whereas those with localized unresectable hepatocellular carcinoma have a five-year survival rate of less than 1%.

Current therapies for hepatitis C include interferon-α (INF_{α}) and combination therapy with ribavirin and interferon. See, e.g., Beremguer et al. (1998) Proc. Assoc. Am. Physicians 110(2):98-112. These therapies suffer from a low sustained response rate and frequent side effects. See, e.g., Hoofnagle et al. (1997) N. Engl. J. Med. 336:347. Currently, no vaccine is available for HCV infection.

### SUMMARY OF THE INVENTION

There remains a need for new treatments and therapies for HCV infection, as well as HCV-associated disorders. There is also a need for compounds useful in the treatment or prevention or amelioration of one or more symptoms of HCV, as well as a need for methods of treatment or prevention or amelioration of one or more symptoms of HCV. Furthermore, there is a need for methods for modulating the activity of HCV-serine proteases, particularly the HCV NS3/NS4a serine protease, using the compounds provided herein.

In one aspect, the invention provides compounds of the Formula I: 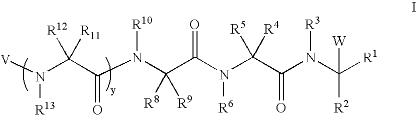
and pharmaceutically acceptable salts and stereoisomers thereof.

In one embodiment, the invention provides a method of treating an HCV-associated disorder comprising administering to a subject in need thereof a pharmaceutically acceptable amount of a compound of the invention, such that the HCV-associated disorder is treated.

In another embodiment, the invention provides a method of treating an HIV infection comprising administering to a subject in need thereof a pharmaceutically acceptable amount of a compound of the invention.

In still another embodiment, the invention provides a method of treating, inhibiting or preventing the activity of HCV in a subject in need thereof, comprising administering to the subject a pharmaceutically acceptable amount of a compound of the invention. In one embodiment, the compounds of the invention inhibit the activity of the NS2 protease, the NS3 protease, the NS3 helicase, the NS5a protein, and/or the NS5b polymerase. In another embodiment, the interaction between the NS3 protease and NS4A cofactor is disrupted. In yet another embodiment, the compounds of the invention prevent or alter the severing of one or more of the NS4A-NS4B, NS4B-NS5A and NS5A-NS5B junctions of the HCV. In another embodiment, the invention provides a method of inhibiting the activity of a serine protease, comprising the step of contacting said serine protease with a compound of the invention. In another embodiment, the invention provides a method of treating, inhibiting or preventing the activity of HCV in a subject in need thereof, comprising administering to the subject a pharmaceutically acceptable amount of a compound of the invention, wherein the compound interacts with any target in the HCV life cycle. In one embodiment, the target of the HCV life cycle is selected from the group consisting of NS2 protease, NS3 protease, NS3 helicase, NS5a protein and NS5b polymerase.

In another embodiment, the invention provides a method of decreasing the HCV RNA load in a subject in need thereof comprising administering to the subject a pharmaceutically acceptable amount of a compound of the invention.

In another embodiment, the compounds of the invention exhibit HCV protease activity. In one embodiment, the compounds are an HCV NS3-4A protease inhibitor.

In another embodiment, the invention provides a method of treating an HCV-associated disorder in a subject, comprising administering to a subject in need thereof a pharmaceutically acceptable amount of a compound of the invention, and a pharmaceutically acceptable carrier, such that the HCV-associated disorder is treated.

In still another embodiment, the invention provides a method of treating an HCV-associated disorder comprising administering to a subject in need thereof a pharmaceutically effective amount of a compound of the invention, in combination with a pharmaceutically effective amount of an additional HCV-modulating compound, such as interferon or derivatized interferon, or a cytochrome P450 monooxygenase inhibitor, such that the HCV-associated disorder is treated. In one embodiment, the additional HCV-modulating compound is selected from the group consisting of Sch 503034 and VX-950.

In another embodiment, the invention provides a method of inhibiting hepatitis C virus replication in a cell, comprising contacting said cell with a compound of the invention.

In yet another embodiment, the invention provides a packaged HCV-associated disorder treatment, comprising an HCV-modulating compound of the invention, packaged with instructions for using an effective amount of the HCV-modulating compound to treat an HCV-associated disorder.

In certain embodiments, the HCV-associated disorder is selected from the group consisting of HCV infection, liver cirrhosis, chronic liver disease, hepatocellular carcinoma, cryoglobulinaemia, non-Hodgkin's lymphoma, and a suppressed innate intracellular immune response.

In another embodiment, the invention provides a method of treating HCV infection, liver cirrhosis, chronic liver disease, hepatocellular carcinoma, cryoglobulinaemia, non-Hodgkin's lymphoma, and/or a suppressed innate intracellular immune response in subject in need thereof comprising administering to the subject a pharmaceutically acceptable amount of a compound of the invention.

In one embodiment, the HCV to be treated is selected of any HCV genotype. In another embodiment, the HCV is selected from HCV genotype 1, 2 and/or 3.

### DETAILED DESCRIPTION OF THE INVENTION

This invention is directed to compounds, e.g., peptide compounds, and intermediates thereto, as well as pharmaceutical compositions containing the compounds for use in treatment of HCV infection. This invention is also directed to the compounds of the invention or compositions thereof as protease inhibitors, particularly as serine protease inhibitors, and more particularly as HCV NS3 protease inhibitors. The compounds are particularly useful in interfering with the life cycle of the hepatitis C virus and in treating or preventing an HCV infection or physiological conditions associated therewith. The present invention is also directed to methods of combination therapy for inhibiting HCV replication in cells, or for treating or preventing an HCV infection in patients using the compounds of the invention or pharmaceutical compositions, or kits thereof.

In one aspect, the invention provides compounds of the Formula I: 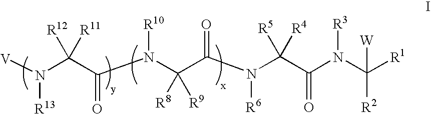
and pharmaceutically acceptable salts and stereoisomers thereof, wherein

x is 0 or 1;

y is 0 or 1;

R¹, R², R⁴, R⁵, R⁶, W, R¹³ and V are each, independently, selected from hydrogen or from the group consisting of alkyl, aralkylaralkyl, heteroalkyl, heterocyclyl, heteroaryl, aryl-heteroaryl, alkyl-heteroaryl, cycloalkyl, alkyloxy, aralkylaralkyloxy, aryloxy, heteroaryloxy, heterocyclyloxy, cycloalkyloxy, amino, mono-and di-alkylamino, arylamino, aralkylamino, heteroarylamino, cycloalkylamino, carboxyalkylamino, arlylalkyloxy and heterocyclylamino; each of which may be further independently substituted one or more times with X¹ and X²; wherein X¹ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl; wherein X¹ can be independently substituted with one or more of X² moieties which can be the same or different and are independently selected; wherein X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;

W is also selected from the group consisting of C(O)OH, C(O)OR²⁴, C(O)-amine, C(O)—C(O)OH, C(═N—O—R²⁴)—C(O)-amine, C(O)N(H)S(O)₂R²⁴, C(O)—C(O)-amine, CON(H)SO₂-amine and C(O)—[C(O)]ₐ-heterocycle, wherein the heterocycle may be substituted or unsubstituted, wherein a is 0 or 1, wherein each R²⁴ is independently selected from the group consisting of H, halogen, —O—, C(O), amino, substituted or unsubstituted-C₁₋₄-alkyl, substituted or unsubstituted-C₃₋₆-cycloalkylC₀₋₄alkyl, substituted or unsubstituted aryl and substituted or unsubstituted heterocycle, and any combination thereof,

V is also selected from the group consisting of -Q¹-Q², wherein Q¹ is absent, C(O), N(H), N(C₁₋₄-alkyl), C═N(CN), C═N(SO₂CH₃), or C═N—COH, and Q² is H or is selected from the group consisting of C₁₋₄-alkyl, O—C₁₋₄-alkyl, NH₂, N(H)—C₁₋₄-alkyl, N(C₁₋₄-alkyl)₂, SO₂-aryl, SO₂-C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄-alkyl substituted by one or more halogen atoms, or C₃₋₆-cycloalkyl;

R³, R⁸, R⁹, R¹⁰, R¹¹ and R¹³ are each, independently, selected from the group consisting of H, C₁₋₄-alkyl and C₃₋₆-cycloalkylC₀₋₄alkyl; and

R¹² is selected from the group consisting of H, C₁₋₄-alkyl and C₃₋₆-cycloalkylC₀₋₄alkyl and aryl;

or R¹ and R² may together form a 3, 4, 5, 6 or 7-membered ring that is aromatic or non-aromatic and may contain one or more heteroatoms, wherein the ring may be further substituted one or more times;

or R¹¹ and V may together form a 3, 4, 5, 6 or 7-membered ring that is aromatic or non-aromatic and may contain one or more additional heteroatoms, wherein the ring may be further substituted one or more times;

or when x and y are 0, R⁶ and V may together form a 3, 4, 5, 6 or 7-membered ring that is aromatic or non-aromatic and may contain one or more additional heteroatoms, wherein the ring may be further substituted one or more times.

In one embodiment of Formula I, y is 0 or 1;

R¹ is selected from the group consisting of H and C₁₋₄-alkyl;

R² is selected from the group consisting of C₁₋₄-alkyl, C(O)C₁₋₄-alkyl, C(O)OC₁₋₄-alkyl, and C₃₋₆cycloalkylC₀₋₄alkyl;

or R¹ and R² may together form a 3, 4, 5, 6 or 7-membered ring that is aromatic or non-aromatic and may contain one or more heteroatoms, wherein the ring may be further substituted one or more times;

W is also selected from the group consisting of C(O)OH, C(O)OR²⁴, C(O)-amine, C(O)—C(O)OH, C(═N—O—R²⁴)—C(O)-amine, C(O)N(H)S(O)₂R²⁴, C(O)—C(O)-amine, CON(H)SO₂-amine and C(O)—[C(O)]ₐ-heterocycle, wherein the heterocycle may be substituted or unsubstituted, wherein a is 0 or 1, wherein each R²⁴ is independently selected from the group consisting of H, halogen, hydroxy, COOH, amino, C(O)NH₂, C₁₋₄-alkyl, C₃₋₆-cycloalkylC₀₋₄alkyl, C₃₋₆-cycloalkylC₀₋₄alkoxy, mono- and diC₁₋₄alkylamino, aryl, aryloxy, aralkyl, aralkyloxy, heterocycleC₀₋₄alkyl, and heterocycleC₀₋₄alkoxy;

R³ is selected from the group consisting of H and C₁₋₄-alkyl;

R⁴ and R⁶ are each, independently, selected from hydrogen or from the group consisting of C₁₋₄-alkyl, C₃₋₆-cycloalkyl, C₃₋₆cycloalkylC₀₋₄alkyl, aryl, aralkylaralkyl and heterocycle, each of which may be independently substituted one or more times;

R⁵ is H;

R⁸, R¹⁰ and R¹¹ are each, independently, selected from the group consisting of H and C₁₋₄-alkyl;

R¹³ are H;

R⁹ is selected from the group consisting of H, C₁₋₄-alkyl and C₃₋₆-cycloalkyl;

R¹² is selected from the group consisting of H, C₁₋₄-alkyl, C₃₋₆-cycloalkyl and aryl; and

V is selected from the group consisting of -Q¹-Q², wherein Q¹ is absent, C(O), S(O)₂, N(H), N(C₁₋₄-alkyl), C═N(CN), C═N(SO₂CH₃), C═N—COH, or C═N—COC₁₋₄alkyl, and Q² is H or is selected from the group consisting of C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄-alkyl substituted by one or more halogen atoms, or C₃₋₆-cycloalkyl;

or R¹¹ and V form the following 5-membered ring which may be further substituted:

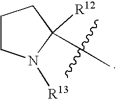

In another embodiment of Formula I, R¹¹ and V form the following structure:

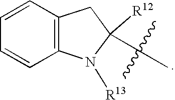

In yet another embodiment of Formula I, R¹⁰ is C(O)C₁₋₄-alkyl. In still another embodiment of Formula I, R¹² is

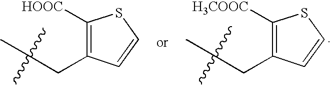

In another embodiment of Formula I, R⁶ is selected from the group consisting of H, cyclopentylmethyl, cyclopropylmethyl, cyclopentyl, cyclopropyl and benzyl. In another embodiment, R¹² is selected from the group consisting of t-butyl and cyclohexyl. In still another embodiment, R⁸ is selected from the group consisting of H and t-butyl.

In another embodiment, Formula I is represented by a compound of the Formula II: 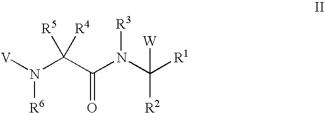
wherein R¹, R², R³, R⁴, R⁵, R⁶, W and V have the meanings set forth for Formula I.

In one embodiment of Formula II, R⁴ and R⁵ are H. In another embodiment of Formula II, V is —C(O)CH₃ or

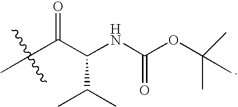

In another embodiment of Formula II, R⁶ is CH₂-cyclopentyl or CH₂-naphthyl. In another embodiment, R⁶ and V form together the following 6-membered ring:

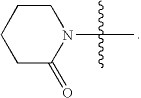

In another embodiment of Formula II, R² is selected from the group consisting of pentyl and CH₂-cyclobutyl.

In one embodiment of the compounds of the invention, R² is selected from the group consisting of propyl and 2-cyclobutyl-ethyl. In another embodiment, R¹¹ is H and R¹² is C₃₋₆-cycloalkyl.

In another embodiment of the compounds of the invention, W, R¹ and R² form a substituent of the following formulas: 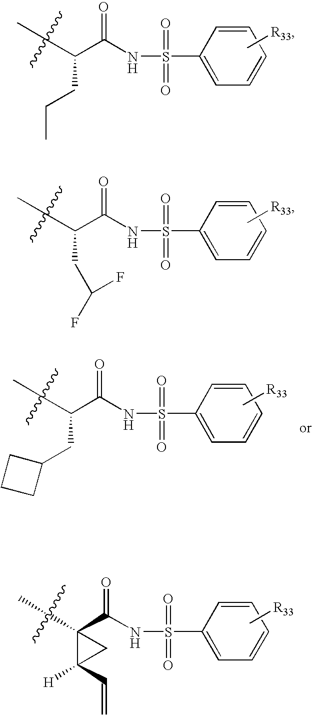
wherein R³³ is selected from the group consisting of H, phenyl, methyl, CF₃, tBu, NO₂, Cl, CN, NH₂, OH, NHCH₃, NHCH₂CH₃, NHCH(CH₃)₂, OCH₃, NHPh, OPh, NHCOCH₃, NHCOPh, OCH2Ph, COCH₃, CO₂Et, CO₂CH₃, CONHPh and CONHCH₃, or R³³ can be a ring fused which taken in combination with the phenyl ring form a naphthyl ring system or a indolyl ring system.

In yet another embodiment of the compounds of the invention, W, R¹ and R² form substituents selected from the group consisting of

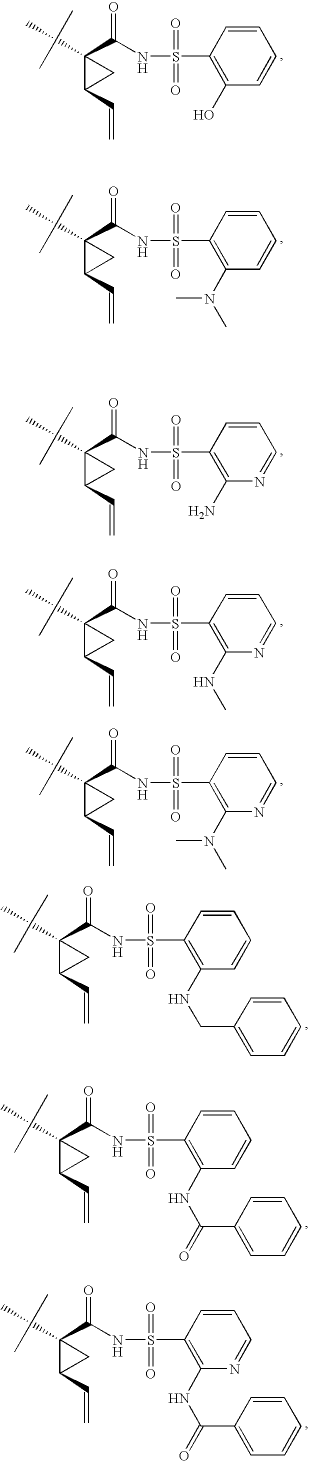

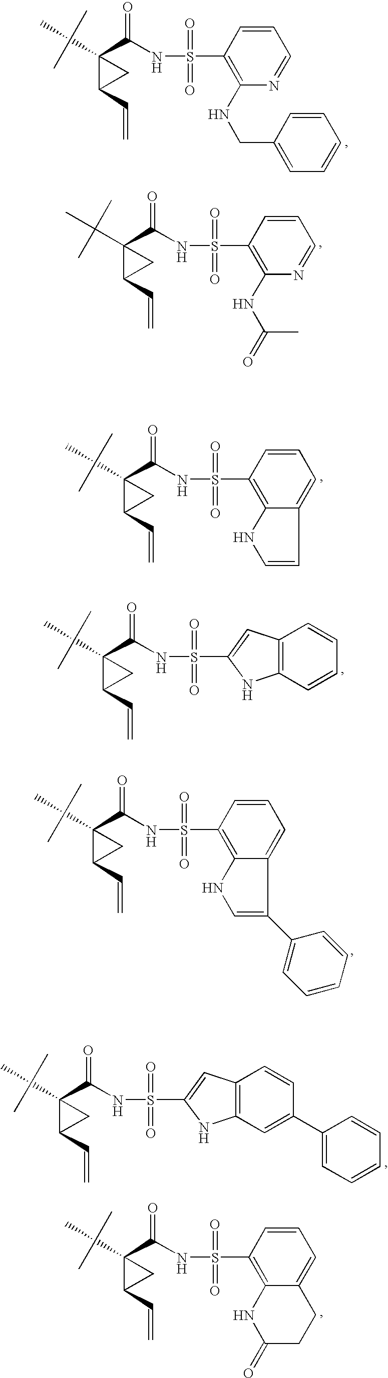

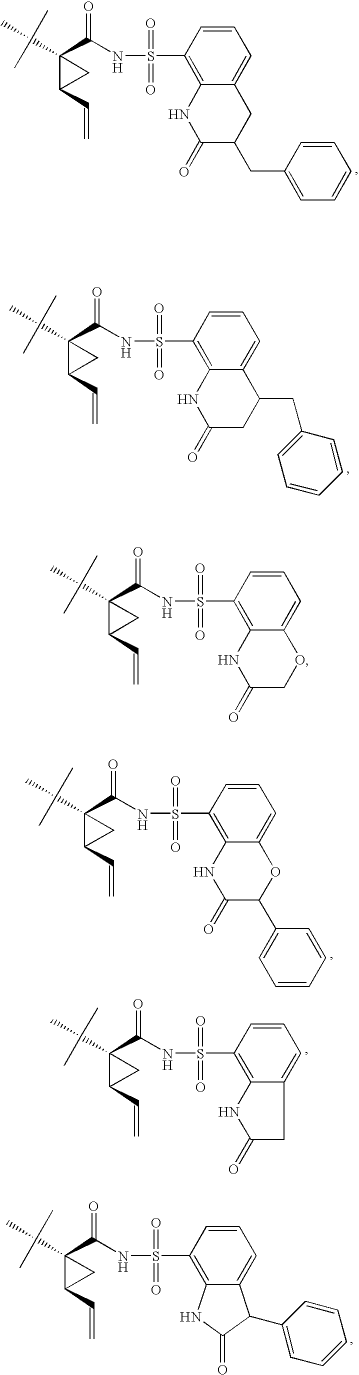

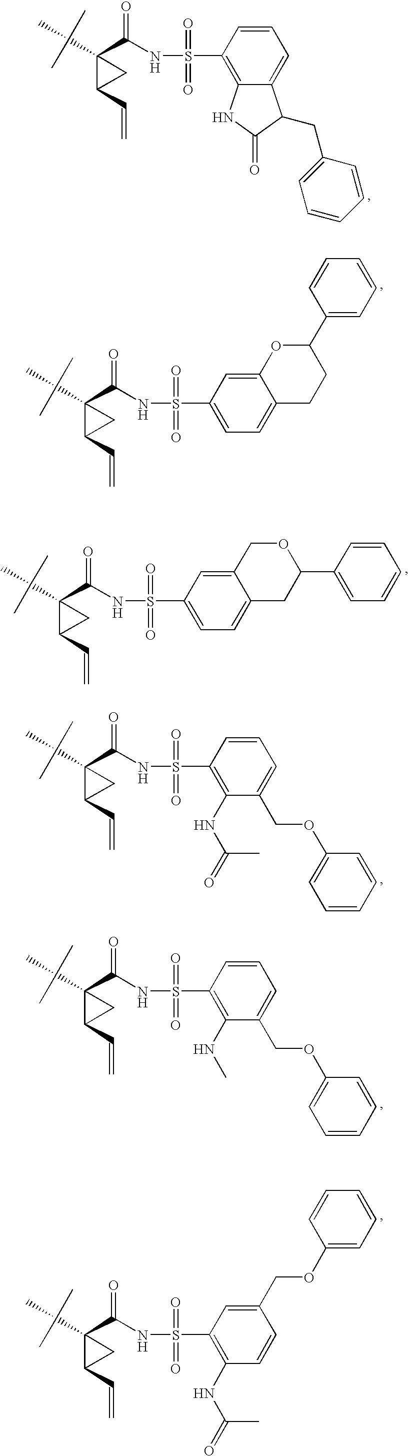

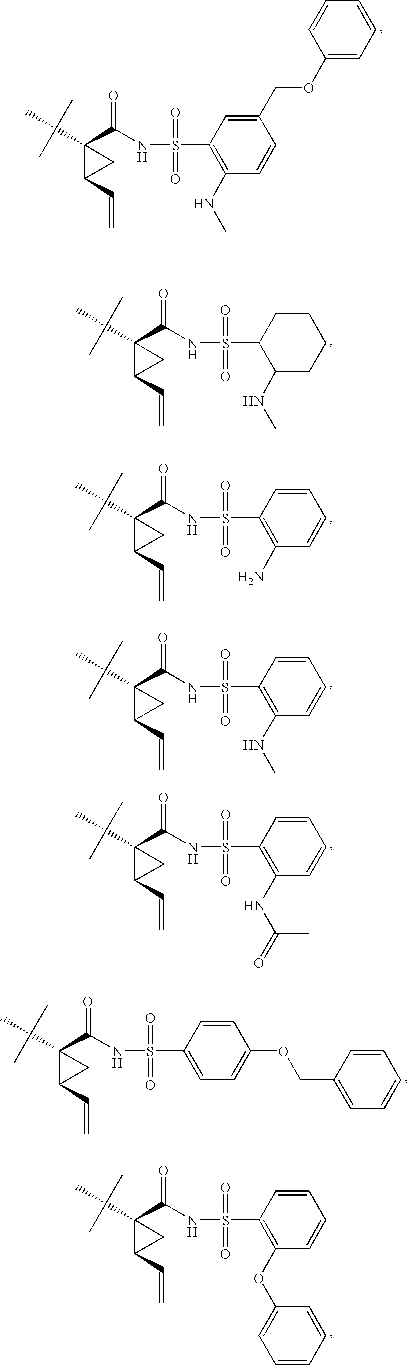

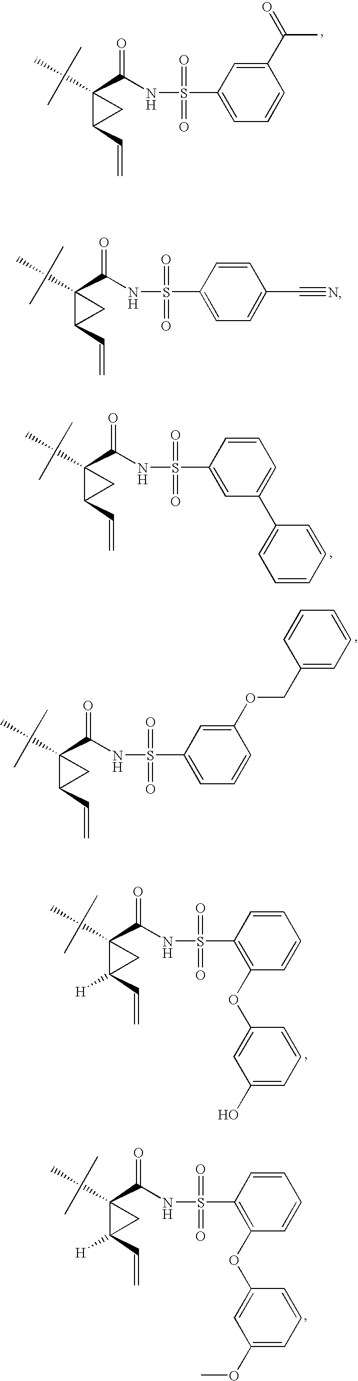

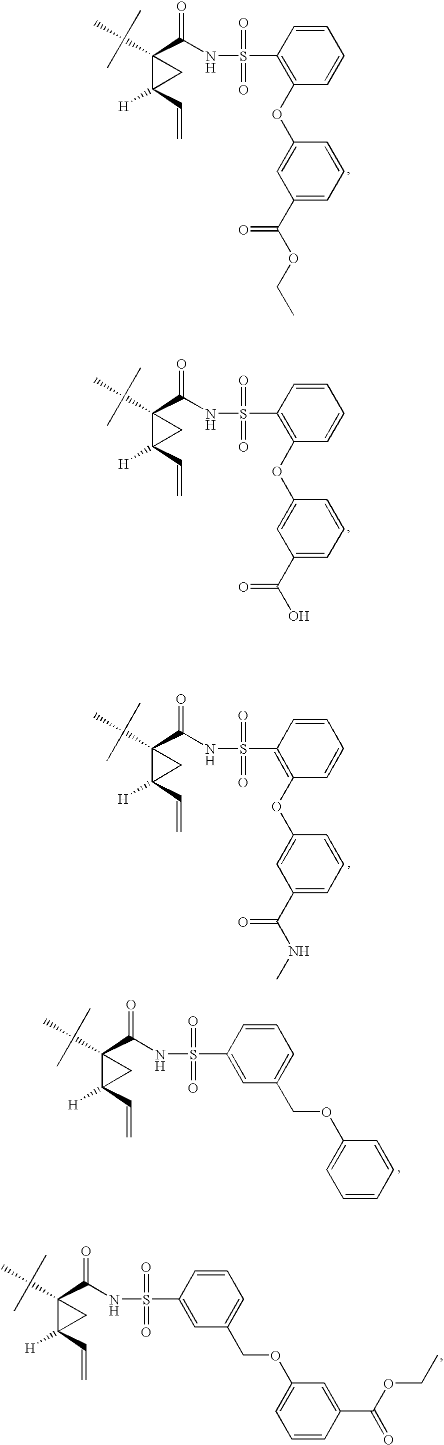

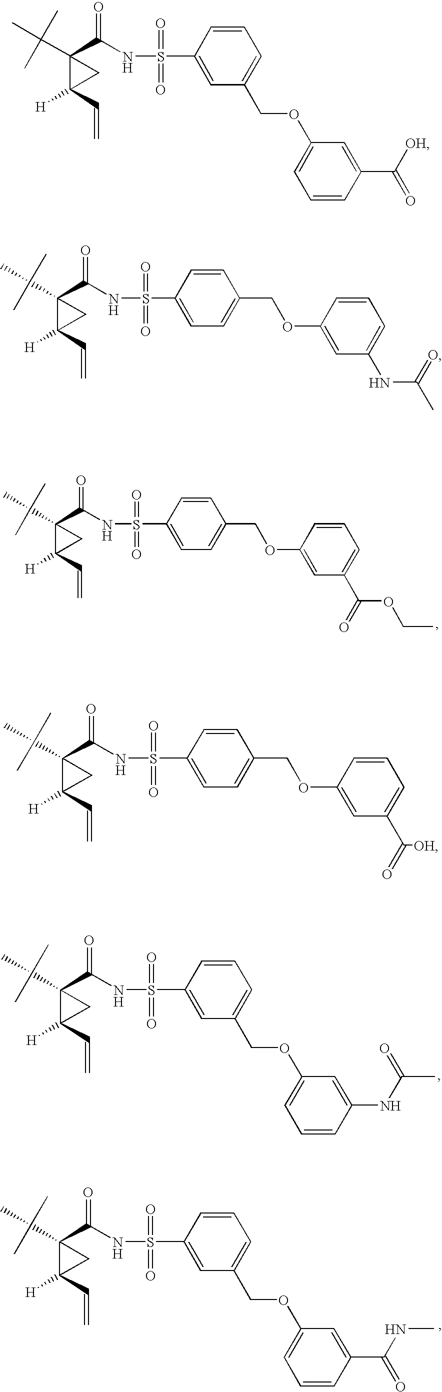

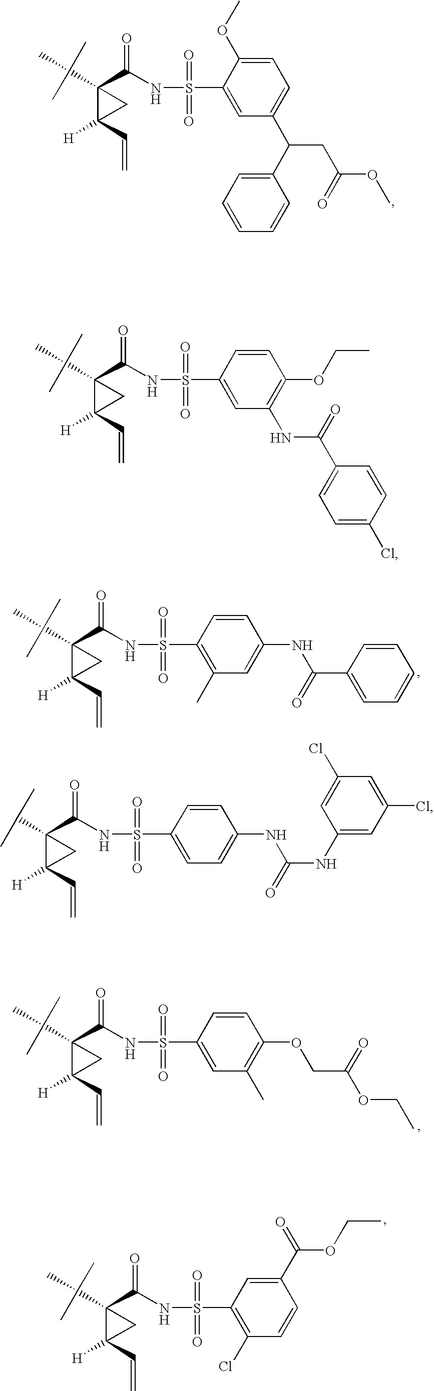

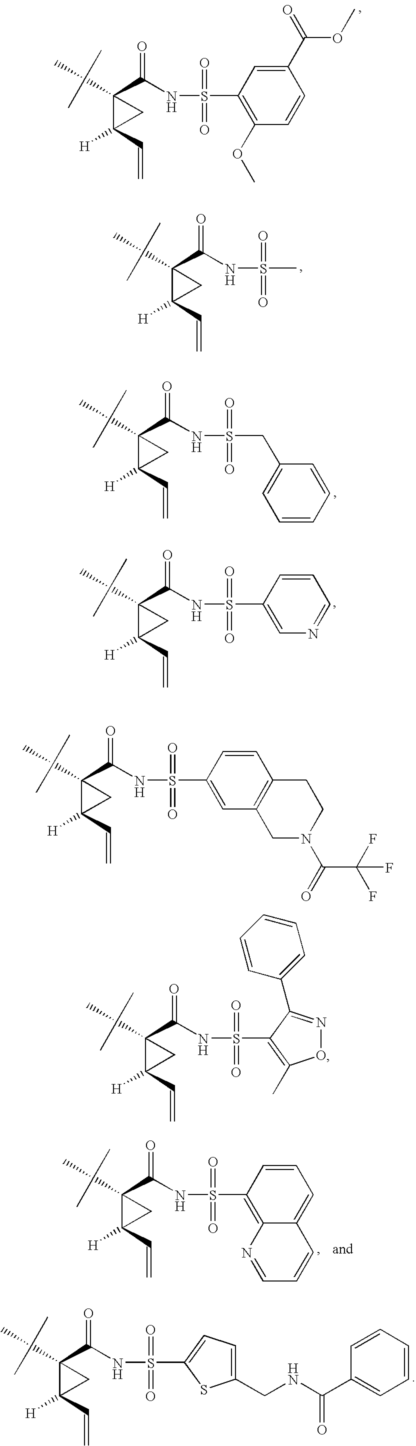

In another embodiment of the compounds of the invention, any of the heterocycle groups are independently selected from the group consisting of acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, each of which may be independently further substituted one or more times with a halogen atom, C₁₋₄-alkyl, C₁₋₄-alkyl substituted by one or more halogen atoms, or C₃₋₆-cycloalkyl.

In another embodiment of the compounds of the invention, W is C(O)—C(O)—N(H)-cyclopropyl. In yet another embodiment of the compounds of the invention, V is selected from the group consisting of C(O)R²⁴, C(O)N(H)R²⁴ and C(O)OR²⁴, wherein each R²⁴ is independently selected from hydrogen or from the group consisting of R²⁴ is independently selected from the group consisting of H, halogen, hydroxy, COOH, amino, C(O)NH₂, C₁₋₄-alkyl, C₃₋₆-cycloalkylC₀₋₄alkyl, C₃₋₆-cycloalkylC₀₋₄alkoxy, mono- an diC₁₋₄alkylamino, aryl, aryloxy, aralkyl, aralkyloxy, heterocycleC₀₋₄alkyl, and heterocycleC₀₋₄alkoxy.

In still another embodiment of the compounds of the invention, V is selected from the group consisting of benzyl, substituted benzyl, naphthyl, C₁₋₄-alkyl, and

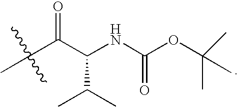

In another embodiment of the compounds of the invention, any of the C₃₋₆-cycloalkyl groups may be independently substituted one or more times with a halogen atom, aryl, trihalomethyl, or C₁₋₄-alkyl. In another embodiment, W is selected from the group consisting of C(O)—C(O)N(R²³)₂, wherein R²³ is independently selected from hydrogen or from the group consisting of C₁₋₄-alkyl, C₃₋₆-cycloalkylC₀₋₄alkyl, aryl and heterocycle, each of which may be independently substituted one or more times with a halogen atom or C₁₋₄-alkyl. In yet another embodiment, W is selected from the group consisting of C(O)—C(O)NH₂, C(O)—C(O)N(H)-cyclopropyl, C(O)-benzothiazole, C(O)-benzoimidazole, C(O)-oxazole, C(O)-imidazole, and C(O)-oxadiazole, wherein the benzothiazole, benzoimidazole, oxazole and oxadiazole groups may be independently substituted one or more times with a halogen atom, aryl, trihalomethyl, C₃₋₆-cycloalkylC₀₋₄alkyl or C₁₋₄-alkyl.

In another embodiment of the compounds of the invention, W is selected from the group consisting of 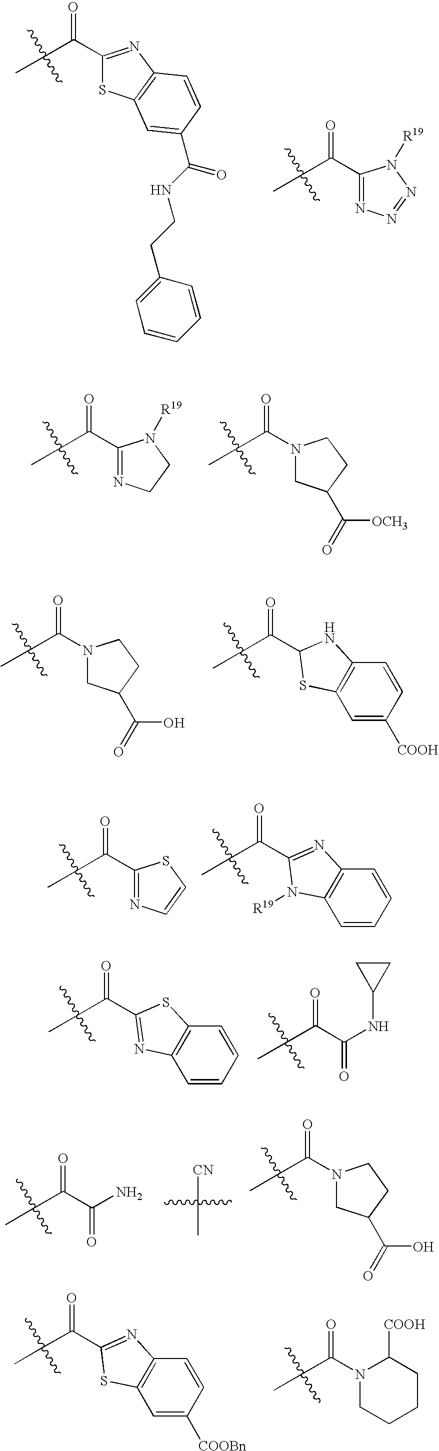 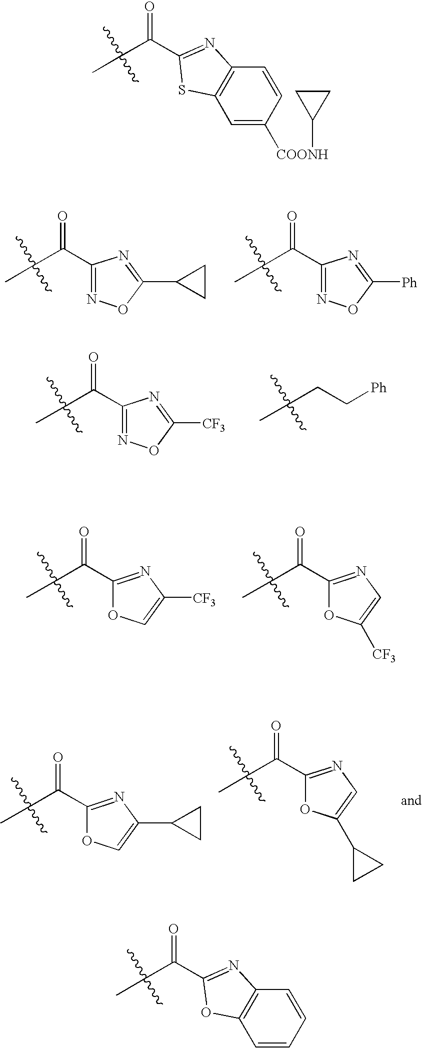
wherein R¹⁹ is selected from the group consisting of hydrogen, a halogen atom, aryl, trihalomethyl, and C₁₋₄-alkyl.

In another embodiment of the compounds of the invention, R² is selected from the group consisting of 2,2-difluoroethyl, propyl, cyclobutyl-methyl and 2-cyclobutyl-ethyl. In another embodiment, R¹¹ is H and R¹² is C₃₋₆-cycloalkyl. In still another embodiment, R¹² is cyclohexyl.

In yet another embodiment of the compounds of the invention, V is selected from the group consisting of C(O)—N(H)-t-butyl. In another embodiment, V is C(O)—R²⁰, wherein R²⁰ is selected from the group consisting of C₃₋₆-cycloalkyl, phenyl, pyrazine, benzooxazole, 4,4-dimethyl-4,5-dihydro-oxazole, benzoimidazole, pyrimidine, benzothiazole 1,1-dioxide and quinazoline, each of which may be further independently substituted with a halogen atom, CF₃, C₁₋₄-alkyl or C₃₋₆-cycloalkyl. In another embodiment, V is C(O)—R²⁰, wherein R²⁰ is selected from the group consisting of 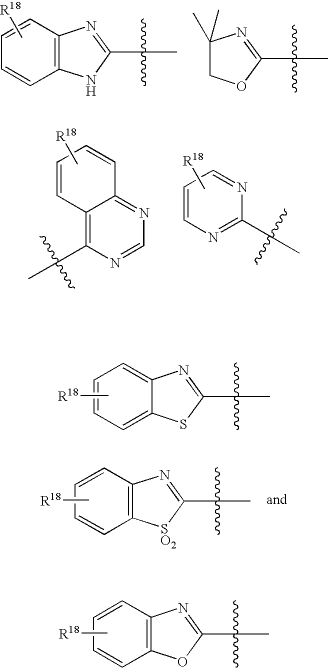
wherein R¹⁸ is selected from the group consisting of hydrogen, a halogen atom, aryl, trihalomethyl, and C₁₋₄-alkyl In still another embodiment, V is C(O)—R²⁰, wherein R²⁰ is selected from the group consisting of 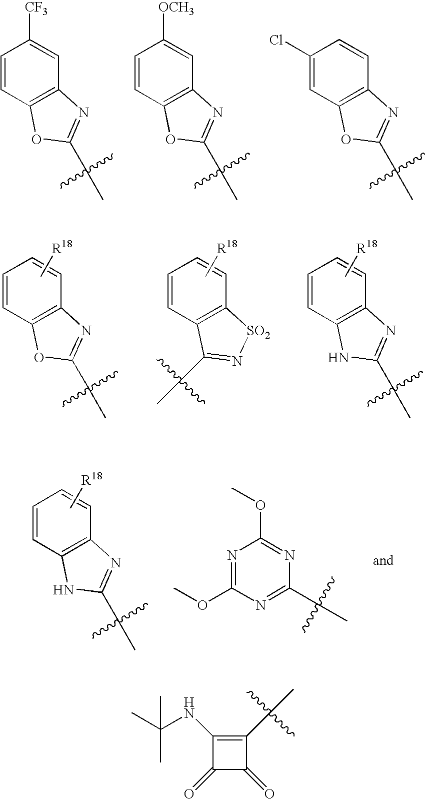
wherein R¹⁸ is selected from the group consisting of hydrogen, a halogen atom, aryl, trihalomethyl, and C₁₋₄-alkyl.

In another embodiment of the compounds of the invention, V is selected from the group consisting of C₃₋₆-cycloalkyl, phenyl, pyrazine, benzooxazole, 4,4-dimethyl-4,5-dihydro-oxazole, benzoimidazole, pyrimidine, benzothiazole 1,1-dioxide and quinazoline, each of which may be further independently substituted with a halogen atom, CF₃, C₁₋₄-alkyl or C₃₋₆-cycloalkyl. In another embodiment, V is selected from the group consisting of 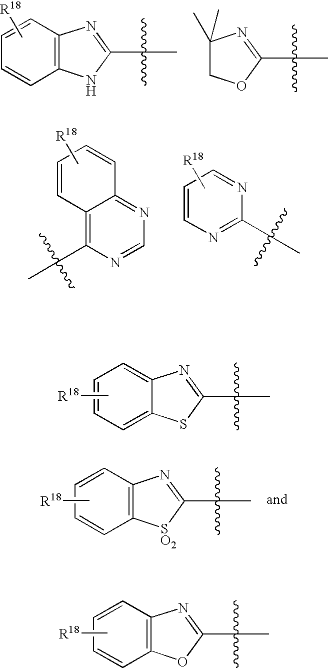
wherein R¹⁸ is selected from the group consisting of hydrogen, a halogen atom, aryl, trihalomethyl, and C₁₋₄-alkyl.

In another embodiment of the compounds of the invention, V is selected from the group consisting of 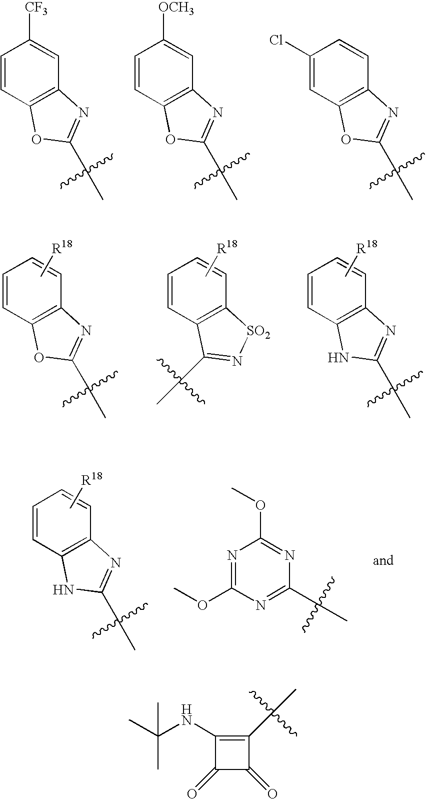
wherein R¹⁸ is selected from the group consisting of hydrogen, a halogen atom, aryl, trihalomethyl, and C₁₋₄-alkyl.

In yet another embodiment of the compounds of the invention, W is C(O)—C(O)-amino. In still another embodiment of the compounds of the invention, R¹³ is H and V is selected from the group consisting of C═N(H)NH₂, C═N(CN)NH₂ and C(O)NH₂. In another embodiment, W is C(O)N(H)S(O)₂R²⁴, wherein R²⁴ is hydrogen or is selected from the group consisting of C₁₋₄-alkyl, C₃₋₆-cycloalkylC₀₋₄alkyl, substituted or unsubstituted aryl and substituted or unsubstituted heterocycle, each of which may be independently substituted one or more times with a halogen atom or C₁₋₄-alkyl. In still another embodiment, W is COOH, R¹ is H, and R² is selected from the group consisting of propyl, 2,2-difluoroethyl and CH₂-cyclobutyl, or R¹ and R² form together a cyclopropyl group that may be further substituted with a vinyl group.

In another embodiment of the compounds of the invention, R¹ and R² form a substituent of the following Formula:

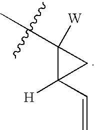

In another embodiment of the compounds of the invention, W, R¹ and R² form a substituent of the following formula:

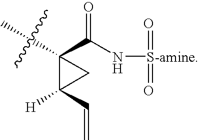

In still another embodiment of the compounds of the invention, W, R¹ and R² form a substituent of the following formula: 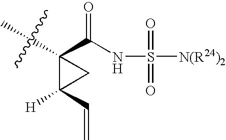
wherein each R²⁴ is independently selected from the group consisting of H, substituted or unsubstituted-C₁₋₄-alkyl, substituted or unsubstituted C₃₋₆-cycloalkylC₀₋₄alkyl, substituted or unsubstituted aryl and substituted or unsubstituted heterocycle.

In another embodiment of the compounds of the invention, R²⁴ is selected from the group consisting of

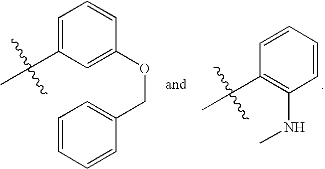

In yet another embodiment of the compounds of the invention, W, R¹ and R² form a substituent selected from the group consisting of:

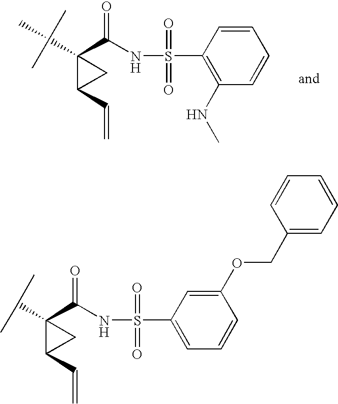

In still another embodiment of the compounds of the invention, V is selected from the group consisting of acyl, SO₂—R²⁴, C(O)N(R²⁴ )₂, C(O)O(R²⁴)₂, and N(H)R²⁴, wherein each R²⁴ is independently selected from the group consisting of H, halogen, hydroxy, COOH, amino, C(O)NH₂, C₁₋₄-alkyl, C₃₋₆-cycloalkylC₀₋₄alkyl, C₃₋₆-cycloalkylC₀₋₄alkoxy, mono- and diC₁₋₄alkylamino, aryl, aryloxy, aralkyl, aralkyloxy, heterocycleC₀₋₄alkyl, and heterocycleC₀₋₄alkoxy.

Preferred embodiments of the compounds of the invention (including pharmaceutically acceptable salts thereof, as well as enantiomers, stereoisomers, rotamers, tautomers, diastereomers, or racemates thereof) are shown below in Table A, Table B, and Table C, and are also considered to be “compounds of the invention.”

The Rest of This Page Intentionally Left Blank

TABLE A

| | Compound |
|---|---|
| Structure | No. |
| 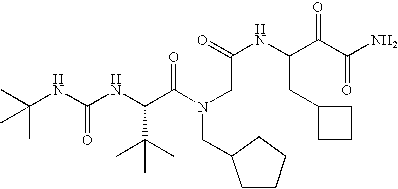 | A-1 |
| 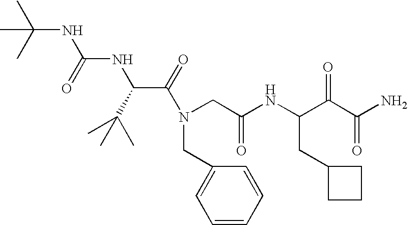 | A-2 |
| 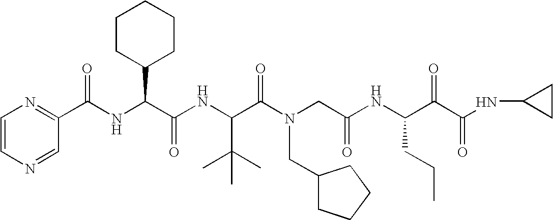 | A-3 |
| 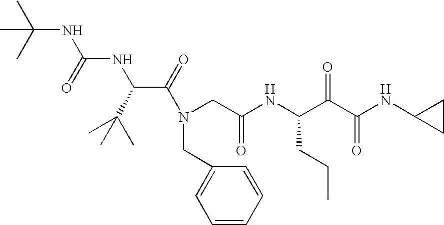 | A-4 |
| 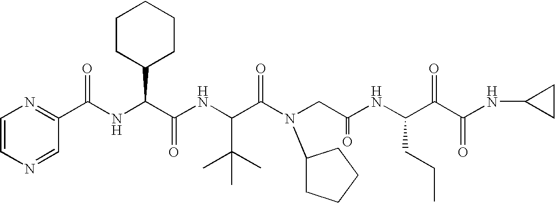 | A-5 |
| 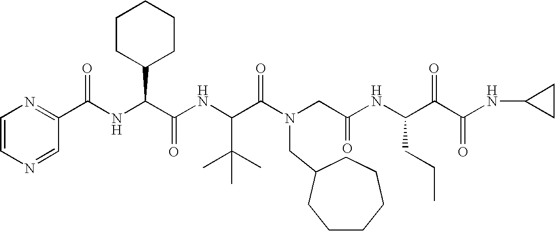 | A-6 |
| 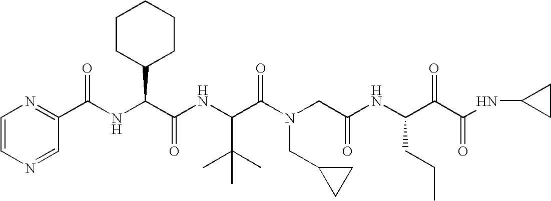 | A-7 |
| 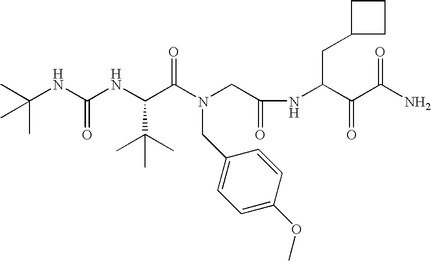 | A-8 |
| 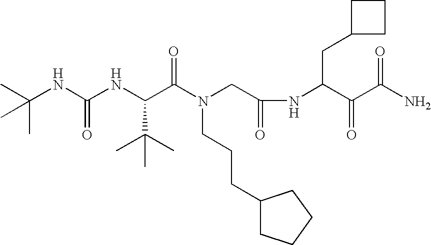 | A-9 |
| 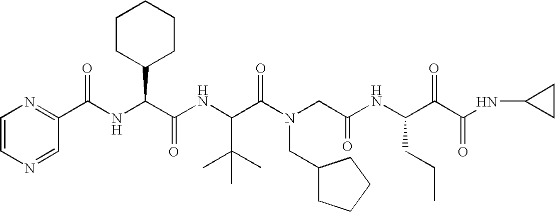 | A-10 |
| 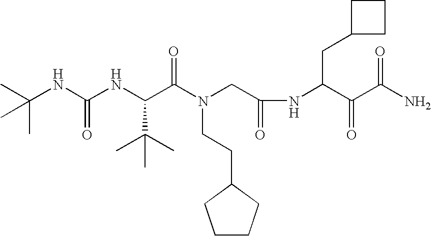 | A-11 |
| 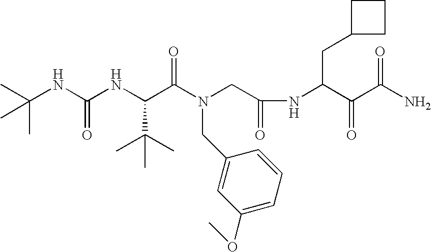 | A-12 |
| 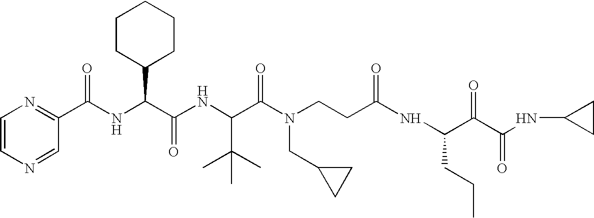 | A-13 |
| 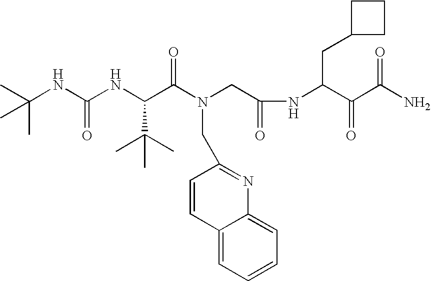 | A-14 |
| 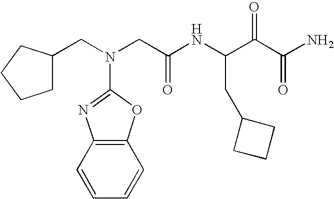 | A-15 |
| 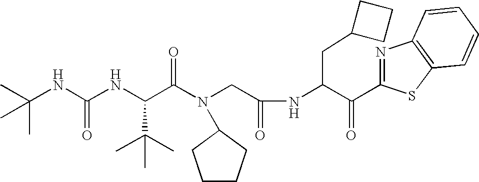 | A-16 |
| 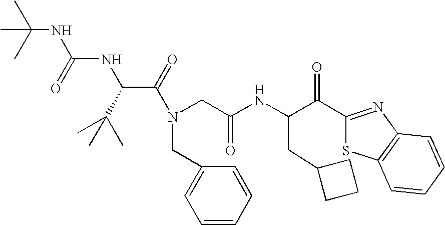 | A-17 |
| 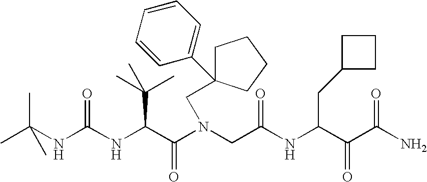 | A-18 |
| 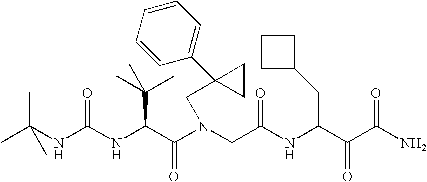 | A-19 |
| 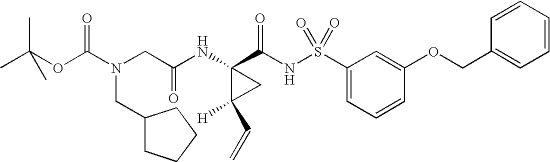 | A-20 |
| 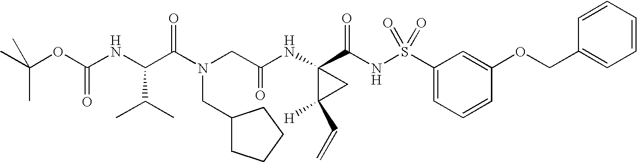 | A-21 |
| 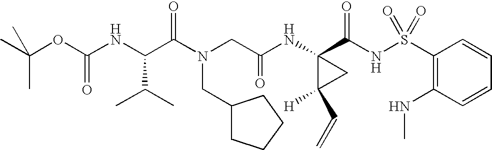 | A-22 |
| 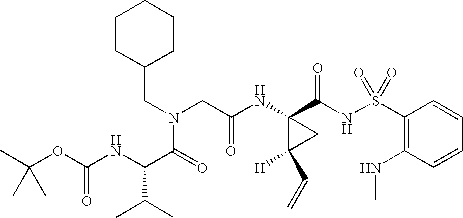 | A-23 |
| 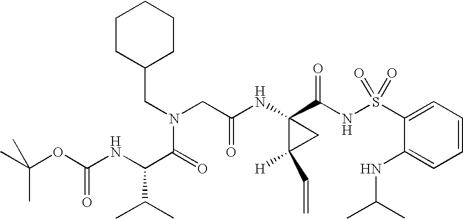 | A-24 |
| 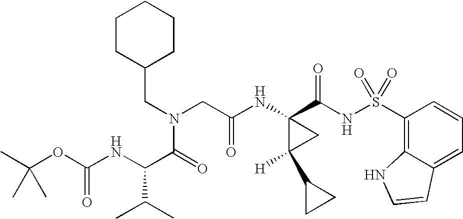 | A-25 |
| 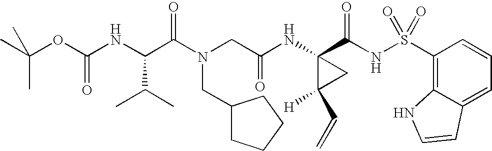 | A-26 |

TABLE B
(R2 = alkyl, aryl)

| | |
|---|---|
| 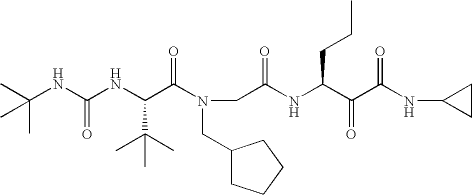 | B-1 |
| 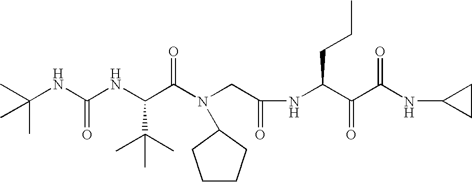 | B-2 |
| 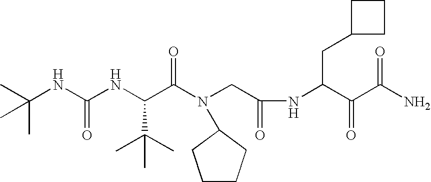 | B-3 |
| 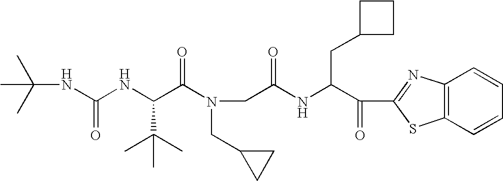 | B-4 |
| 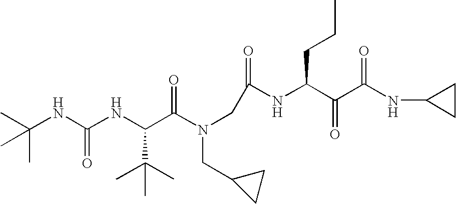 | B-5 |
| 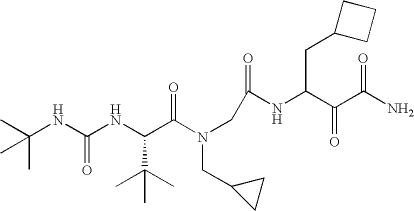 | B-6 |
| 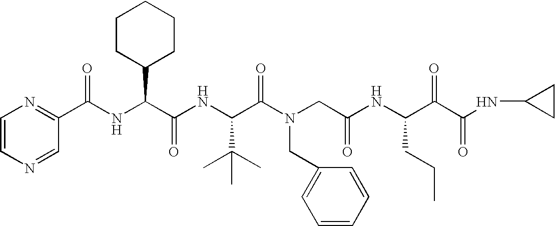 | B-7 |
| 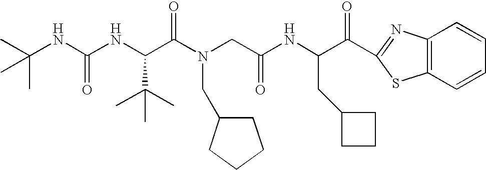 | B-8 |
| 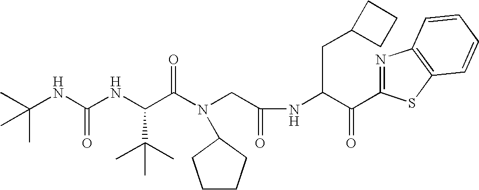 | B-9 |
| 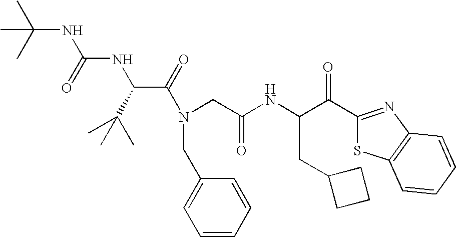 | B-10 |
| 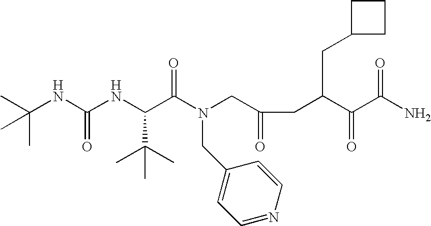 | B-11 |
| 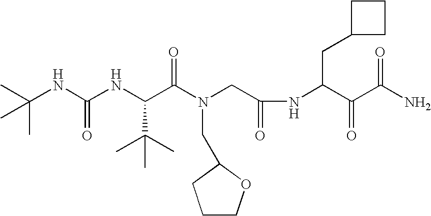 | B-12 |
| 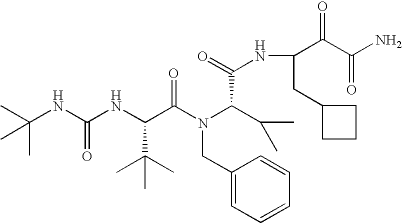 | B-13 |
| 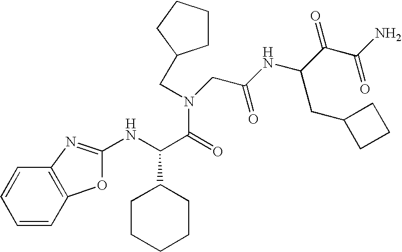 | B-14 |
| 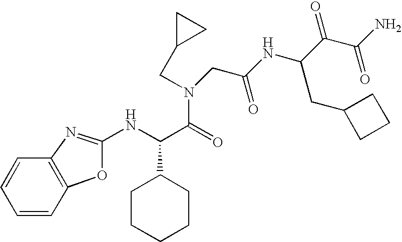 | B-15 |
| 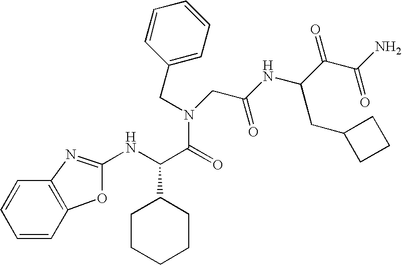 | B-16 |
| 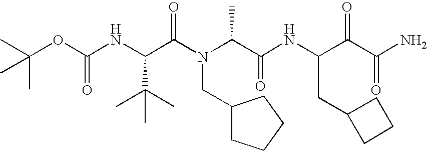 | B-17 |
| 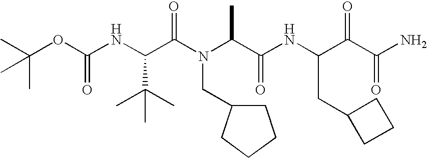 | B-18 |
| 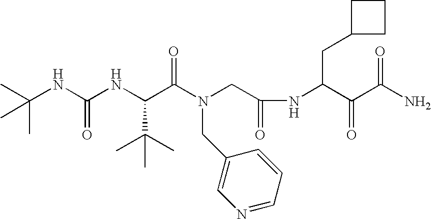 | B-19 |
| 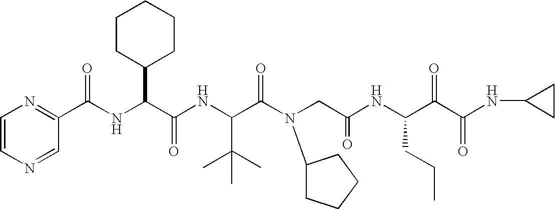 | B-20 |
| 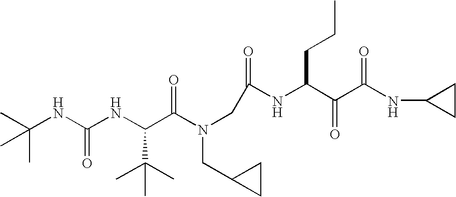 | B-21 |
| 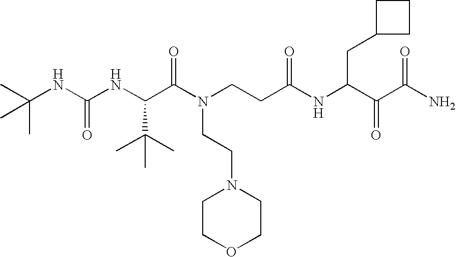 | B-22 |
| 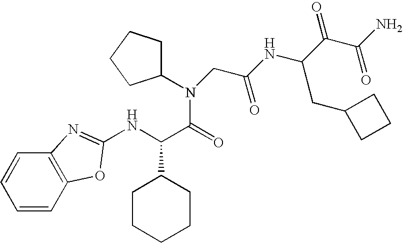 | B-23 |
| 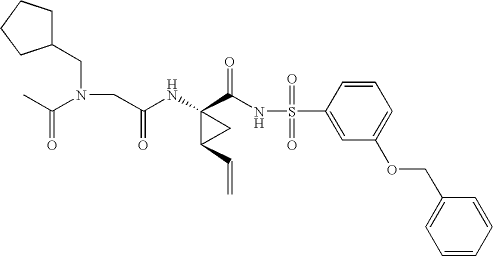 | B-24 |
| 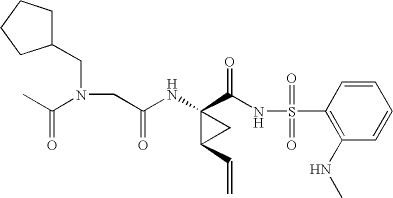 | B-25 |
| 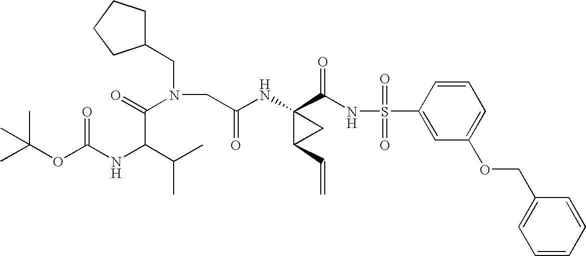 | B-26 |
| 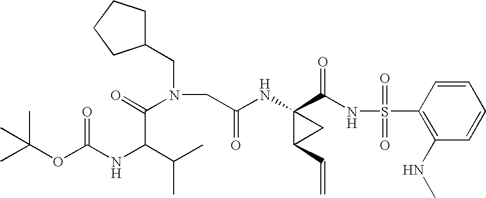 | B-27 |
| 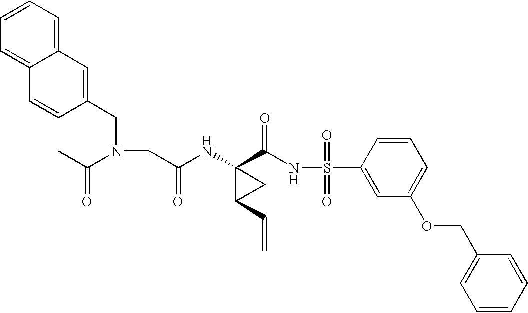 | B-28 |
| 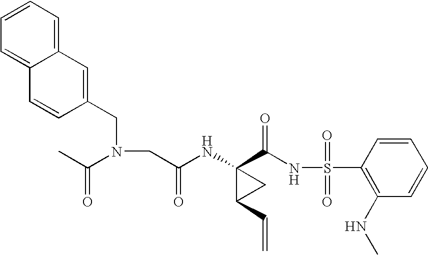 | B-29 |
| 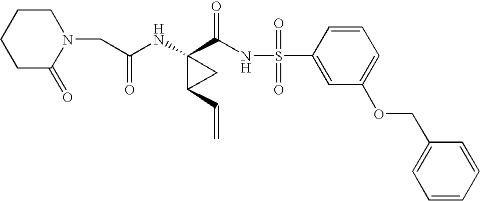 | B-30 |
| 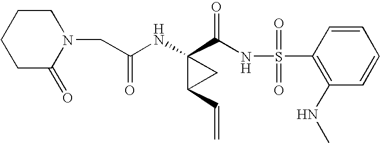 | B-31 |
| 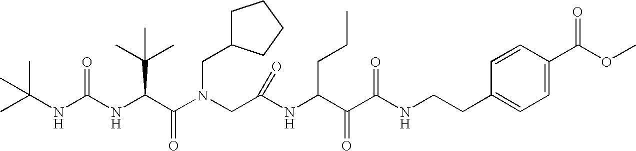 | B-32 |
| 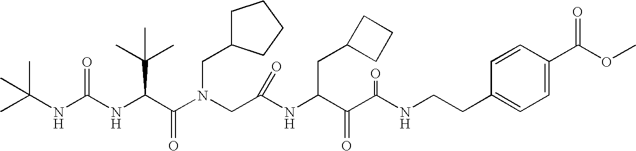 | B-33 |

Using the HCV NS3-4A protease and Luciferase-HCV replicon assays described in the exemplification section below, the compounds of the invention (including compounds of Table A depicted above) are found to show IC₅₀ values for HCV inhibition in the range from 10 to more than 100 μM, or 5.0 to 30 μM, including, for example, the range from 2.0 to 10 μM or less.

In certain embodiments, a compound of the present invention is further characterized as a modulator of HCV, including a mammalian HCV, and especially including a human HCV. In a preferred embodiment, the compound of the invention is an HCV inhibitor.

In certain embodiments, the compound of the invention is not VX-950 or Sch 503034 (see, e.g. Curr. Med. Chem., 2005, 12, 2317-2342; and Antimicrob Agents Chemother. March 2006;50(3): 1013-20, both of which are incorporated herein by reference in their entirety).

In other embodiments, the compounds of the invention are not the species described in International Patent Application Nos. WO 2005/058821, WO/2005/021584, WO/01/18369, WO/03/062265, WO/02/18369, WO/2003/087092 and U.S. Pat. App. No. 2002/0032175.

The terms “HCV-associated state” or “HCV-associated disorder” include disorders and states (e.g., a disease state) that are associated with the activity of HCV, e.g., infection of HCV in a subject. HCV-associated states include HCV-infection, liver cirrhosis, chronic liver disease, hepatocellular carcinoma, cryoglobulinaemia, non-Hodgkin's lymphoma, and a suppressed innate intracellular immune response.

HCV-associated states are often associated with the NS3 serine protease of HCV, which is responsible for several steps in the processing of the HCV polyprotein into smaller functional proteins. NS3 protease forms a heterodimeric complex with the NS4A protein, an essential cofactor that enhances enzymatic activity, and is believed to help anchor HCV to the endoplasmic reticulum. NS3 first autocatalyzes hydrolysis of the NS3-NS4A juncture, and then cleaves the HCV polyprotein intermolecularly at the NS4A-NS4B, NS4B-NS5A and NS5A-NS5B intersections. This process is associated with replication of HCV in a subject. Inhibiting or modulating the activity of one or more of the NS3, NS4A, NS4B, NS5A and NS5B proteins will inhibit or modulate replication of HCV in a subject, thereby preventing or treating the HCV-associated state. In a particular embodiment, the HCV-associated state is associated with the activity of the NS3 protease. In another particular embodiment, the HCV-associated state is associated with the activity of NS3-NS4A heterodimeric complex.

In one embodiment, the compounds of the invention are NS3/NS4A protease inhibitors. In another embodiment, the compounds of the invention are NS2/NS3 protease inhibitors.

Without being bound by theory, it is believed that the disruption of the above protein-protein interactions by the compounds of the invention will interfere with viral polyprotein processing by the NS3 protease and thus viral replication.

HCV-associated disorders also include HCV-dependent diseases. HVC-dependent diseases include, e.g., any disease or disorder that depend on or related to activity or misregulation of at least one strain of HCV.

The present invention includes treatment of HCV-associated disorders as described above, but the invention is not intended to be limited to the manner by which the compound performs its intended function of treatment of a disease. The present invention includes treatment of diseases described herein in any manner that allows treatment to occur, e.g. HCV infection.

In a related embodiment, the compounds of the invention can be useful for treating diseases related to HIV, as well as HIV infection and AIDS (Acquired Immune Deficiency Syndrome).

In certain embodiments, the invention provides a pharmaceutical composition of any of the compounds of the present invention. In a related embodiment, the invention provides a pharmaceutical composition of any of the compounds of the present invention and a pharmaceutically acceptable carrier or excipient of any of these compounds. In certain embodiments, the invention includes the compounds as novel chemical entities.

In one embodiment, the invention includes a packaged HCV-associated disorder treatment. The packaged treatment includes a compound of the invention packaged with instructions for using an effective amount of the compound of the invention for an intended use.

The compounds of the present invention are suitable as active agents in pharmaceutical compositions that are efficacious particularly for treating HCV-associated disorders. The pharmaceutical composition in various embodiments has a pharmaceutically effective amount of the present active agent along with other pharmaceutically acceptable excipients, carriers, fillers, diluents and the like. The phrase, “pharmaceutically effective amount” as used herein indicates an amount necessary to administer to a host, or to a cell, issue, or organ of a host, to achieve a therapeutic result, especially an anti-HCV effect, e.g., inhibition of proliferation of the HCV virus, or of any other HCV-associated disease.

In one embodiment, the diseases to be treated by compounds of the invention include, for example, HCV infection, liver cirrhosis, chronic liver disease, hepatocellular carcinoma, cryoglobulinaemia, non-Hodgkin's lymphoma, and a suppressed innate intracellular immune response.

In other embodiments, the present invention provides a method for inhibiting the activity of HCV. The method includes contacting a cell with any of the compounds of the present invention. In a related embodiment, the method further provides that the compound is present in an amount effective to selectively inhibit the activity of one or more of the NS3, NS4A, NS4B, NS5A and NS5B proteins. In another related embodiment, the method provides that the compound is present in an amount effective to diminish the HCV RNA load in a subject.

In other embodiments, the present invention provides a use of any of the compounds of the invention for manufacture of a medicament to treat HCV infection in a subject.

In other embodiments, the invention provides a method of manufacture of a medicament, including formulating any of the compounds of the present invention for treatment of a subject.

### DEFINITIONS

The term “treat,” “treated,” “treating” or “treatment” includes the diminishment or alleviation of at least one symptom associated or caused by the state, disorder or disease being treated. In certain embodiments, the treatment comprises the induction of an HCV-inhibited state, followed by the activation of the HCV-modulating compound, which would in turn diminish or alleviate at least one symptom associated or caused by the HCV-associated state, disorder or disease being treated. For example, treatment can be diminishment of one or several symptoms of a disorder or complete eradication of a disorder.

The term “subject” is intended to include organisms, e.g., prokaryotes and eukaryotes, which are capable of suffering from or afflicted with an HCV-associated disorder. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from an HCV-associated disorder, and for diseases or conditions described herein, e.g. HCV infection. In another embodiment, the subject is a cell.

The language “HCV-modulating compound,” “modulator of HCV” or “HCV inhibitor” refers to compounds that modulate, e.g., inhibit, or otherwise alter, the activity of HCV. Similarly, an “NS3/NS4A protease inhibitor,” or an “NS2/NS3 protease inhibitor” refers to a compound that modulates, e.g., inhibits, or otherwise alters, the interaction of these proteases with one another. Examples of HCV-modulating compounds include compounds of Formula I, as well as Table A and Table B (including pharmaceutically acceptable salts thereof, as well as enantiomers, stereoisomers, rotamers, tautomers, diastereomers, or racemates thereof).

Additionally, the method includes administering to a subject an effective amount of an HCV-modulating compound of the invention, e.g., HCV-modulating compounds of Formula I, as well as Table A and Table B (including pharmaceutically acceptable salts thereof, as well as enantiomers, stereoisomers, rotamers, tautomers, diastereomers, or racemates thereof).

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term “alkyl” also includes alkenyl groups and alkynyl groups. Furthermore, the expression “Cₓ-C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl and isobutyl. Moreover, the term C₃₋₆-cycloalkyl includes, but is not limited to, cyclopropyl, cyclopentyl, and cyclohexyl. As discussed below, these alkyl groups, as well as cycloalkyl groups, may be further substituted.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer carbons. Likewise, preferred cycloalkyls have from 4-7 carbon atoms in their ring structure, and more preferably have 5 or 6 carbons in the ring structure.

Moreover, alkyl (e.g., methyl, ethyl, propyl, butyl, pentyl, hexyl, etc.) include both “unsubstituted alkyl” and “substituted alkyl”, the latter of which refers to alkyl moieties having substituents replacing a hydrogen on one or more carbons of the hydrocarbon backbone, which allow the molecule to perform its intended function.

The term “substituted” is intended to describe moieties having substituents replacing a hydrogen on one or more atoms, e.g. C, O or N, of a molecule. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, morpholino, phenol, benzyl, phenyl, piperizine, cyclopentane, cyclohexane, pyridine, 5H-tetrazole, triazole, piperidine, or an aromatic or heteroaromatic moiety.

Further examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g. benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g. —CN), —NO₂, halogen (e.g., —F, —Cl, —Br, or —I), (CR′R″)₀₋₃C(halogen)₃ (e.g. —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g. —OH), (CR′R″)₀₋₃COR′, (CR′R″)₀₋₃ (substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. In certain embodiments, a carbonyl moiety (C═O) may be further derivatized with an oxime moiety, e.g. an aldehyde moiety may be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

The term “alkenyl” includes unsaturated aliphatic groups analogous in length and possible substitution to the alkyls described above, but which contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g. ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “amine” or “amino” should be understood as being broadly applied to both a molecule, or a moiety or functional group, as generally understood in the art, and may be primary, secondary, or tertiary. The term “amine” or “amino” includes compounds where a nitrogen atom is covalently bonded to at least one carbon, hydrogen or heteroatom. The terms include, for example, but are not limited to, “alkylamino,” “arylamino,” “diarylamino,” “alkylarylamino,” “alkylaminoaryl,” “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

The term “aryl” includes groups, including 5- and 6-membered single-ring aromatic groups that may include from zero to four heteroatoms, for example, phenyl, pyrrole, furan, thiophene, thiazole, isothiaozole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, anthryl, phenanthryl, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “carbonyl” or “carboxy” includes compounds and moieties which contain a carbon connected with a double bond to an oxygen atom, and tautomeric forms thereof. Examples of moieties that contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g. ureas) are also include as well as other combinations of carbonyl groups bound to heteroatoms (e.g. nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “thiocarbonyl” or “thiocarboxy” includes compounds and moieties which contain a carbon connected with a double bond to a sulfur atom. The term “thiocarbonyl moiety” includes moieties that are analogous to carbonyl moieties. For example, “thiocarbonyl” moieties include aminothiocarbonyl, wherein an amino group is bound to the carbon atom of the thiocarbonyl group, furthermore other thiocarbonyl moieties include, oxythiocarbonyls (oxygen bound to the carbon atom), aminothiocarbonylamino groups, etc.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom that is covalently bonded to another alkyl group.

The term “ester” includes compounds and moieties that contain a carbon or a heteroatom bound to an oxygen atom that is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom that is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” include moieties with two or more rings (e.g., cycloalkyls, cycloalkenyls, cycloalkynyls, aryls and/or heterocyclyls) in which two or more carbons are common to two adjoining rings, e.g. the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

Additionally, the phrase “any combination thereof” implies that any number of the listed functional groups and molecules may be combined to create a larger molecular architecture. For example, the terms “phenyl,” “carbonyl” (or “═O”), “—O—,” “—OH,” and C₁₋₆ (i.e., —CH₃ and —CH₂CH₂CH₂—) can be combined to form a 3-methoxy-4-propoxybenzoic acid substituent. It is to be understood that when combining functional groups and molecules to create a larger molecular architecture, hydrogens can be removed or added, as required to satisfy the valence of each atom.

It is to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two bonds.

It will be noted that the structures of some of the compounds of this invention include asymmetric carbon atoms. It is to be understood accordingly that the isomers arising from such asymmetry (e.g., all enantiomers, stereoisomers, rotamers, tautomers, diastereomers, or racemates) are included within the scope of this invention. Such isomers can be obtained in substantially pure form by classical separation techniques and by stereochemically controlled synthesis. Furthermore, the structures and other compounds and moieties discussed in this application also include all tautomers thereof. Compounds described herein may be obtained through art recognized synthesis strategies.

It will also be noted that the substituents of some of the compounds of this invention include isomeric cyclic structures. It is to be understood accordingly that constitutional isomers of particular substituents are included within the scope of this invention, unless indicated otherwise. For example, the term “tetrazole” includes tetrazole, 2H-tetrazole, 3H-tetrazole, 4H-tetrazole and 5H-tetrazole.

Use in HCV-Associated Disorders

The compounds of the present invention have valuable pharmacological properties and are useful in the treatment of diseases. In certain embodiments, compounds of the invention are useful in the treatment of HCV-associated disorders, e.g., as drugs to treat HCV infection.

The term “use” includes any one or more of the following embodiments of the invention, respectively: the use in the treatment of HCV-associated disorders; the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise. In particular, diseases to be treated and are thus preferred for use of a compound of the present invention are selected from HCV-associated disorders, including those corresponding to HCV-infection, as well as those diseases that depend on the activity of one or more of the NS3, NS4A, NS4B, NS5A and NS5B proteins, or a NS3-NS4A, NS4A-NS4B, NS4B-NS5A or NS5A-NS5B complex. The term “use” further includes embodiments of compositions herein which bind to an HCV protein sufficiently to serve as tracers or labels, so that when coupled to a fluor or tag, or made radioactive, can be used as a research reagent or as a diagnostic or an imaging agent.

In certain embodiments, a compound of the present invention is used for treating HCV-associated diseases, and use of the compound of the present invention as an inhibitor of any one or more HCVs. It is envisioned that a use can be a treatment of inhibiting one or more strains of HCV.

Assays

The inhibition of HCV activity may be measured as using a number of assays available in the art. An example of such an assay can be found in Anal Biochem. 1996 240(1): 60-7; which is incorporated by reference in its entirety. Assays for measurement of HCV activity are also described in the experimental section below.

Pharmaceutical Compositions

The language “effective amount” of the compound is that amount necessary or sufficient to treat or prevent an HCV-associated disorder, e.g. prevent the various morphological and somatic symptoms of an HCV-associated disorder, and/or a disease or condition described herein. In an example, an effective amount of the HCV-modulating compound is the amount sufficient to treat HCV infection in a subject. In another example, an effective amount of the HCV-modulating compound is the amount sufficient to treat HCV infection, liver cirrhosis, chronic liver disease, hepatocellular carcinoma, cryoglobulinaemia, non-Hodgkin's lymphoma, and a suppressed innate intracellular immune response in a subject. The effective amount can vary depending on such factors as the size and weight of the subject, the type of illness, or the particular compound of the invention. For example, the choice of the compound of the invention can affect what constitutes an “effective amount.” One of ordinary skill in the art would be able to study the factors contained herein and make the determination regarding the effective amount of the compounds of the invention without undue experimentation.

The regimen of administration can affect what constitutes an effective amount. The compound of the invention can be administered to the subject either prior to or after the onset of an HCV-associated state. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation.

Compounds of the invention may be used in the treatment of states, disorders or diseases as described herein, or for the manufacture of pharmaceutical compositions for use in the treatment of these diseases. Methods of use of compounds of the present invention in the treatment of these diseases, or pharmaceutical preparations having compounds of the present invention for the treatment of these diseases.

The language “pharmaceutical composition” includes preparations suitable for administration to mammals, e.g. humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof, and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats an HCV-associated disorder.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

Synthetic Procedure

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group,” unless the context indicates otherwise. The protection of functional groups by such protecting groups, the protecting groups themselves, and their cleavage reactions are described for example in standard reference works, such as e.g. Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known per se. For example, salts of compounds of the present invention having acid groups may be formed, for example, by treating the compounds with metal compounds, such as alkali metal salts of suitable organic carboxylic acids, e.g., the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g., by treating the compounds with an acid or a suitable anion exchange reagent. Internal salts of compounds of the present invention containing acid and basic salt-forming groups, e.g. a free carboxy group and a free amino group, may be formed, e.g., by the neutralisation of salts, such as acid addition salts, to the isoelectric point, e.g. with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by, e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like.

General Process Conditions

The following applies in general to all processes mentioned throughout this disclosure.

The process steps to synthesize the compounds of the invention can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g., in the H⁻ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described in Science of Synthesis: Houben-Weyl Methods of Molecular Transformation. Georg Thieme Verlag, Stuttgart, Germany. 2005.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitrites, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

Pro-Drugs

The present invention also relates to pro-drugs of a compound of the present invention that are converted in vivo to the compounds of the present invention as described herein. Any reference to a compound of the present invention is therefore to be understood as referring also to the corresponding pro-drugs of the compound of the present invention, as appropriate and expedient.

Combinations

A compound of the present invention may also be used in combination with other agents, e.g., an additional HCV-modulating compound that is or is not of the formula I, for treatment of and HCV-associated disorder in a subject.

By the term “combination”, is meant either a fixed combination in one dosage unit form, or a kit of parts for the combined administration where a compound of the present invention and a combination partner may be administered independently at the same time or separately within time intervals that especially allow that the combination partners show a cooperative, e.g. synergistic, effect, or any combination thereof.

For example, WO 2005/042020, incorporated herein by reference in its entirety, describes the combination of various HCV inhibitors with a cytochrome P450 (“CYP”) inhibitor. Any CYP inhibitor that improves the pharmacokinetics of the relevant NS3/4A protease may be used in combination with the compounds of this invention. These CYP inhibitors include, but are not limited to, ritonavir (WO 94/14436, incorporated herein by reference in its entirety), ketoconazole, troleandomycin, 4-methyl pyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methyl pyrazole, cyclosporin, and clomethiazole.

Methods for measuring the ability of a compound to inhibit CYP activity are known (see, e.g. U.S. Pat. No. 6,037,157 and Yun, et al. Drug Metabolism & Disposition, vol. 21, pp. 403-407 (1993); incorporated herein by reference). For example, a compound to be evaluated may be incubated with 0.1, 0.5, and 1.0 mg protein/ml, or other appropriate concentration of human hepatic microsomes (e.g., commercially available, pooled characterized hepatic microsomes) for 0, 5, 10, 20, and 30 minutes, or other appropriate times, in the presence of an NADPH-generating system. Control incubations may be performed in the absence of hepatic microsomes for 0 and 30 minutes (triplicate). The samples may be analyzed for the presence of the compound. Incubation conditions that produce a linear rate of compound metabolism will be used a guide for further studies. Experiments known in the art can be used to determine the kinetics of the compound metabolism (Kₘ and Vₘₐₓ). The rate of disappearance of compound may be determined and the data analyzed according to Michaelis-Menten kinetics by using Lineweaver-Burk, Eadie-Hofstee, or nonlinear regression analysis.

Inhibition of metabolism experiments may then be performed. For example, a compound (one concentration, <Kₘ) may be incubated with pooled human hepatic microsomes in the absence or presence of a CYP inhibitor (such as ritonavir) under the conditions determined above. As would be recognized, control incubations should contain the same concentration of organic solvent as the incubations with the CYP inhibitor. The concentrations of the compound in the samples may be quantitated, and the rate of disappearance of parent compound may be determined, with rates being expressed as a percentage of control activity.

Methods for evaluating the influence of co-administration of a compound of the invention and a CYP inhibitor in a subject are also known (see, e.g., US2004/0028755; incorporated herein by reference). Any such methods could be used in connection with this invention to determine the pharmacokinetic impact of a combination. Subjects that would benefit from treatment according to this invention could then be selected.

Accordingly, one embodiment of this invention provides a method for administering an inhibitor of CYP3A4 and a compound of the invention. Another embodiment of this invention provides a method for administering an inhibitor of isozyme 3A4 (“CYP3A4”), isozyme 2C19 (“CYP2C19”), isozyme 2D6 (“CYP2D6”), isozyme 1A2 (“CYP1A2”), isozyme 2C9 (“CYP2C9”), or isozyme 2E1 (“CYP2E1”). In embodiments where the protease inhibitor is VX-950 (or a sterereoisomer thereof), the CYP inhibitor preferably inhibits CYP3A4.

As would be appreciated, CYP3A4 activity is broadly observed in humans. Accordingly, embodiments of this invention involving inhibition of isozyme 3A4 would be expected to be applicable to a broad range of patients.

Accordingly, this invention provides methods wherein the CYP inhibitor is administered together with the compound of the invention in the same dosage form or in separate dosage forms.

The compounds of the invention (e.g., compound of Formula I or subformulae thereof) may be administered as the sole ingredient or in combination or alteration with other antiviral agents, especially agents active against HCV. In combination therapy, effective dosages of two or more agents are administered together, whereas in alternation or sequential-step therapy, an effective dosage of each agent is administered serially or sequentially. In general, combination therapy is typically preferred over alternation therapy because it induces multiple simultaneous stresses on the virus. The dosages given will depend on absorption, inactivation and excretion rate of the drug as well as other factors. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. The efficacy of a drug against the viral infection can be prolonged, augmented, or restored by administering the compound in combination or alternation with a second, and perhaps third antiviral compound that induces a different gene mutation than that caused by the principle drug in a drug resistant virus. Alternatively, the pharmacokinetic, biodistribution or other parameters of the drug can be altered by such combination or alternation therapy.

Daily dosages required in practicing the method of the present invention will vary depending upon, for example, the compound of the invention employed, the host, the mode of administration, the severity of the condition to be treated. A preferred daily dosage range is about from 1 to 50 mg/kg per day as a single dose or in divided doses. Suitable daily dosages for patients are on the order of from e.g. 1 to 20 mg/kg p.o or i.v. Suitable unit dosage forms for oral administration comprise from ca. 0.25 to 10 mg/kg active ingredient, e.g. compound of Formula I or any subformulae thereof, together with one or more pharmaceutically acceptable diluents or carriers therefor. The amount of co-agent in the dosage form can vary greatly, e.g., 0.00001 to 1000 mg/kg active ingredient.

Daily dosages with respect to the co-agent used will vary depending upon, for example, the compound employed, the host, the mode of administration and the severity of the condition to be treated. For example, lamivudine may be administered at a daily dosage of 100 mg. The pegylated interferon may be administered parenterally one to three times per week, preferably once a week, at a total weekly dose ranging from 2 to 10 million IU, more preferable 5 to 10 million IU, most preferable 8 to 10 million IU. Because of the diverse types of co-agent that may be used, the amounts can vary greatly, e.g., 0.0001 to 5,000 mg/kg per day.

The current standard of care for treating hepatitis C is the combination of pegylated interferon alpha with ribavirin, of which the recommended doses are 1.5 pg/kg/wk peginterferon alfa-2b or 180 μg/wk peginterferon alfa-2a, plus 1,000 to 1,200 mg daily of ribavirin for 48 weeks for genotype I patients, or 800 mg daily of ribavirin for 24 weeks for genotype ⅔ patients.

The compound of the invention (e.g., compound of Formula I or subformulae thereof) and co-agents of the invention may be administered by any conventional route, in particular enterally, e.g. orally, for example in the form of solutions for drinking, tablets or capsules or parenterally, for example in the form of injectable solutions or suspensions. Certain preferred pharmaceutical compositions may be e.g. those based on microemulsions as described in UK 2,222,770 A.

The compound of the invention (e.g., compound of Formula I or subformulae thereof) are administered together with other drugs (co-agents) e.g. a drug which has anti-viral activity, especially anti-Flaviviridae activity, most especially anti-HCV activity, e.g. an interferon, e.g. interferon-α-2a or interferon-α-2b, e.g. Intron^{R} A, Roferon^{R}, Avonex^{R}, Rebif^{R} or Betaferon^{R}, or an interferon conjugated to a water soluble polymer or to human albumin, e.g. albuferon, an anti-viral agent, e.g. ribavirin, lamivudine, the compounds disclosed in U.S. Pat. No. 6,812,219 and WO 2004/002422 A2 (the disclosures of which are incorporated herein by reference in their entireties), an inhibitor of the HCV or other Flaviviridae virus encoded factors like the NS3/4A protease, helicase or RNA polymerase or a prodrug of such an inhibitor, an anti-fibrotic agent, e.g. a N-phenyl-2-pyrimidine-amine derivative, e.g. imatinib, an immune modulating agent, e.g. mycophenolic acid, a salt or a prodrug thereof, e.g. sodium mycophenolate or mycophenolate mofetil, or a SIP receptor agonist, e.g. FTY720 or an analogue thereof optionally phosphorylated, e.g. as disclosed in EP627406A1, EP778263A1, EP1002792A1, WO02/18395, WO02/76995, WO 02/06268, JP2002316985, WO03/29184, WO03/29205, WO03/62252 and WO03/62248, the disclosures of which are incorporated herein by reference in their entireties.

Conjugates of interferon to a water-soluble polymer are meant to include especially conjugates to polyalkylene oxide homopolymers such as polyethylene glycol (PEG) or polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Such interferon-polymer conjugates are described in U.S. Pat. Nos. 4,766,106, 4,917,888, European Patent Application No. 0 236 987, European Patent Application No. 0 510 356 and International Application Publication No. WO 95/13090, the disclosures of which are incorporated herein by reference in their entireties. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

Especially preferred conjugates of interferon are pegylated alfa-interferons, for example pegylated interferon-α-2a, pegylated interferon-α-2b; pegylated consensus interferon or pegylated purified interferon-α product. Pegylated interferon-α-2a is described e.g. in European Patent 593,868 (incorporated herein by reference in its entirety) and commercially available e.g. under the tradename PEGASYS® (Hoffmann-La Roche). Pegylated interferon-α-2b is described, e.g. in European Patent 975,369 (incorporated herein by reference in its entirety) and commercially available e.g. under the tradename PEG-INTRON A® (Schering Plough). Pegylated consensus interferon is described in WO 96/11953 (incorporated herein by reference in its entirety). The preferred pegylated α-interferons are pegylated interferon-α-2a and pegylated interferon-α-2b. Also preferred is pegylated consensus interferon.

Other preferred co-agents are fusion proteins of an interferon, for example fusion proteins of interferon-α-2a, interferon-α-2b; consensus interferon or purified interferon-α product, each of which is fused with another protein. Certain preferred fusion proteins comprise an interferon (e.g., interferon-α-2b) and an albumin as described in U.S. Pat. No. 6,973,322 and international publications WO02/6007 1, WO05/003296 and WO05/077042 (Human Genome Sciences). A preferred interferon conjugated to a human albumin is Albuferon (Human Genome Sciences).

Cyclosporins which bind strongly to cyclophilin but are not immunosuppressive include those cyclosporins recited in U.S. Pat. Nos. 5,767,069 and 5,981,479 and are incorporated herein by reference. Melle⁴-Cyclosporin is a preferred non-immunosuppressive cyclosporin. Certain other cyclosporin derivatives are described in WO2006039668 (Scynexis) and WO2006038088 (Debiopharm SA) and are incorporated herein by reference. A cyclosporin is considered to be non-immunosuppressive when it has an activity in the Mixed Lymphocyte Reaction (MLR) of no more than 5%, preferably no more than 2%, that of cyclosporin A. The Mixed Lymphocyte Reaction is described by T. Meo in “Immunological Methods”, L. Lefkovits and B. Peris, Eds., Academic Press, N.Y. pp. 227-239 (1979). Spleen cells (0.5×10⁶) from Balb/c mice (female, 8-10 weeks) are co-incubated for 5 days with 0.5×10⁶ irradiated (2000 rads) or mitomycin C treated spleen cells from CBA mice (female, 8-10 weeks). The irradiated allogeneic cells induce a proliferative response in the Balb/c spleen cells which can be measured by labeled precursor incorporation into the DNA. Since the stimulator cells are irradiated (or mitomycin C treated) they do not respond to the Balb/c cells with proliferation but do retain their antigenicity. The IC₅₀ found for the test compound in the MLR is compared with that found for cyclosporin A in a parallel experiment. In addition, non-immunosuppressive cyclosporins lack the capacity of inhibiting CN and the downstream NF-AT pathway. [Melle]⁴-ciclosporin is a preferred non-immunosuppressive cyclophilin-binding cyclosporin for use according to the invention.

Ribavirin (1-β-D-ribofuranosyl-1-1,2,4-triazole-3-caroxamide) is a synthetic, non-interferon-inducing, broad spectrum antiviral nucleoside analog sold under the trade name, Virazole (The Merk Index, 11^{th} edition, Editor: Budavar, S, Merck & Co., Inc., Rahway, N.J., p1304,1989). U.S. Pat. No. 3,798,209 and RE29,835 (incorporated herein by reference in their entireties) disclose and claim ribavirin. Ribavirin is structurally similar to guanosine, and has in vitro activity against several DNA and RNA viruses including Flaviviridae (Gary L. Davis, Gastroenterology 118:S 104-S114, 2000).

Ribavirin reduces serum amino transferase levels to normal in 40% of patients, but it does not lower serum levels of HCV-RNA (Gary L. Davis, Gastroenterology 118:S104-S114, 2000). Thus, ribavirin alone is not effective in reducing viral RNA levels. Additionally, ribavirin has significant toxicity and is known to induce anemia. Ribavirin is not approved for monotherapy against HCV; it is approved in combination with interferon alpha-2a or interferon alpha-2b for the treatment of HCV.

A further preferred combination is a combination of a compound of the invention (e.g., a compound of Formula I or any subformulae thereof) with a non-immunosuppressive cyclophilin-binding cyclosporine, with mycophenolic acid, a salt or a prodrug thereof, and/or with a SIP receptor agonist, e.g. FTY720.

Additional examples of compounds that can be used in combination or alternation treatments include:

(1) Interferons, including interferon alpha 2a or 2b and pegylated (PEG) interferon alpha 2a or 2b, for example:
(a) Intron-A®, interferon alfa-2b (Schering Corporation, Kenilworth, N.J.);
(b) PEG-Intron®, peginteferon alfa-2b (Schering Corporation, Kenilworth, N.J.);
(c) Roferon®, recombinant interferon alfa-2a (Hoffmann-La Roche, Nutley, N.J.);
(d) Pegasys®, peginterferon alfa-2a (Hoffmann-La Roche, Nutley, N.J.);
(e) Berefor®, interferon alfa 2 available (Boehringer Ingelheim Pharmaceutical, Inc., Ridgefield, Conn.);
(f) Sumiferon®, a purified blend of natural alpha interferons (Sumitomo, Japan)
(g) Wellferon®, lymphoblastoid interferon alpha n1 (GlaxoSmithKline);
(h) Infergen®, consensus alpha interferon (InterMune Pharmaceuticals, Inc., Brisbane, Calif.);
(i) Alferon®, a mixture of natural alpha interferons (Interferon Sciences, and Purdue Frederick Co., Conn.);
(j) Viraferon®;
(k) Consensus alpha interferon from Amgen, Inc., Newbury Park, Calif.,

Other forms of interferon include: interferon beta, gamma, tau and omega, such as Rebif (Interferon beta 1a) by Serono, Omniferon (natural interferon) by Viragen, REBIF (interferon beta-1a) by Ares-Serono, Omega Interferon by BioMedicines; oral Interferon Alpha by Amarillo Biosciences; an interferon conjugated to a water soluble polymer or to a human albumin, e.g., Albuferon (Human Genome Sciences), an antiviral agent, a consensus interferon, ovine or bovine interferon-tau

Conjugates of interferon to a water-soluble polymer are meant to include especially conjugates to polyalkylene oxide homopolymers such as polyethylene glocol (PEG) or polypropylene glycols, polyoxyethylenated polyols, copolymers thereof and block copolymers thereof. As an alternative to polyalkylene oxid-based polymers, effectively non-antigenic materials such as dextran, polyvinyl pyrrolidones, polyacrylamides, polyvinyl alcohols, carbohydrate-based polymers and the like can be used. Since the polymeric modification sufficiently reduces antigenic response, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

(2) Ribavirin, such as ribavirin (1-beta-D-ribofuranosyl-1H-1,2,4-triazole-3-carboxamide) from Valeant Pharmaceuticals, Inc., Costa Mesa, Calif.); Rebetol® from Schering Corporation, Kenilworth, N.J., and Copegus® from Hoffmann-La Roche, Nutley, N.J.; and new ribavirin analogues in development such as Levovirin and Viramidine by Valeant,

(3) Thiazolidine derivatives which show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193;

(4) Thiazolidines and benzanilides identified in Kakiuchi N. et al. J. FEBS Letters 421, 217-220; Takeshita N. et al. Analytical Biochemistry, 1997, 247, 242-246;

(5) A phenan-threnequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griseofulvum, which demonstrates activity in a scintillation proximity assay (Chu M. et al, Bioorganic and Medicinal Chemistry Letters 9, 1949-1952);

(6) Protease inhibitors.

Examples include substrate-based NS3 protease inhibitors (Attwood et al., Antiviral peptide derivatives, PCT WO 98/22496, 1998; Attwood et al., Antiviral Chemistry and Chemotherapy 1999, 10, 259-273; Attwood et al, Preparation and use of amino acid derivatives as anti-viral agents, German Patent Pub. DE 19914474; Tung et al. Inhibitors of serine proteases, particularly hepatitis C virus NS3 protease; PCT WO 98/17679), including alphaketoamides and hydrazinoureas, and inhibitors that terminate in an electrophile such as a boronic acid or phosphonate (Llinas-Brunet et al. Hepatitis C inhibitor peptide analogues, PCT WO 99/07734) are being investigated.

Non-substrate-based NS3 protease inhibitors such as 2,4,6-trihydroxy-3-nitro-benzamide derivatives (Sudo K. et al., Biochemiscal and Biophysical Research Communications, 1997, 238 643-647; Sudo K. et al. Antiviral Chemistry and Chemotherapy, 1998, 9, 186), including RD3-4082 and RD3-4078, the former substituted on the amide with a 14 carbon chain and the latter processing apara-phenoxyphenyl group are also being investigated.

Sch 68631, a phenanthrenequinone, is an HCV protease inhibitor (Chu M et al., Tetrahedron Letters 37:7229-7232, 1996). In another example by the same authors, Sch 351633, isolated from the fungus Penicillium grieofulvum, was identified as a protease inhibitor (Chu M. et al., Bioorganic and Medicinal Chemistry Letters 9:1949-1952). Nanomolar potency against the HCV NS3 protease enzyme has been achieved by the design of selective inhibitors based on the macromolecule eglin c. Eglin c, isolated from leech, is a potent inhibitor of several serine proteases such as S. griseus proteases A and B, ∀-chymotrypsin, chymase and subtilisin. Qasim M. A. et al., Biochemistry 36:1598-1607, 1997.

U.S. patents disclosing protease inhibitors for the treatment of HCV include, for example, U.S. Pat. No. 6,004,933 to Spruce et al (incorporated herein by reference in its entirety) which discloses a class of cysteine protease inhibitors for inhibiting HCV endopeptidase 2; U.S. Pat. No. 5,990,276 to Zhang et al. (incorporated herein by reference in its entirety) which discloses synthetic inhibitors of hepatitis C virus NS3 protease; U.S. Pat. No. 5,538,865 to Reyes et al. (incorporated herein by reference in its entirety). Peptides as NS3 serine protease inhibitors of HCV are disclosed in WO 02/008251 to Corvas International, Inc., and WO 02/08187 and WO 02/008256 to Schering Corporation (incorporated herein by reference in their entireties). HCV inhibitor tripeptides are disclosed in U.S. Pat. Nos. 6,534,523, 6,410,531 and 6,420,380 to Boehringer Ingelheim and WO 02/060926 to Bristol Myers Squibb (incorporated herein by reference in their entireties). Diaryl peptides as NS3 serine protease inhibitors of HCV are disclosed in WO 02/48172 to Schering Corporation (incorporated herein by reference). Imidazoleidinones as NS3 serine protease inhibitors of HCV are disclosed in WO 02/18198 to Schering Corporation and WO 02/48157 to Bristol Myers Squibb (incorporated herein by reference in their entireties). WO 98/17679 to Vertex Pharmaceuticals and WO 02/48116 to Bristol Myers Squibb also disclose HCV protease inhibitors (incorporated herein by reference in their entireties).

HCV NS3-4A serine protease inhibitors including BILN 2061 by Boehringer Ingelheim, VX-950 by Vertex, SCH 6/7 by Schering-Plough, and other compounds currently in preclinical development;

Substrate-based NS3 protease inhibitors, including alphaketoamides and hydrazinoureas, and inhibitors that terminate in an elecrophile such as a boronic acid or phosphonate; Non-substrate-based NS3 protease inhibitors such as 2,4,6-trihydroxy-3-nitro-benzamide derivatives including RD3-4082 and RD3-4078, the former substituted on the amide with a 14 carbon chain and the latter processing a para-phenoxyphenyl group; and Sch68631, a phenanthrenequinone, an HCV protease inhibitor.

Sch 351633, isolated from the fungus Penicillium griseofulvum was identified as a protease inhibitor. Eglin c, isolated from leech is a potent inhibitor of several serine proteases such as S. griseus proteases A and B, a-chymotrypsin, chymase and subtilisin.

U.S. Pat. No. 6,004,933 (incorporated herein by reference in its entirety) discloses a class of cysteine protease inhibitors from inhibiting HCV endopeptidase 2; synthetic inhibitors of HCV NS3 protease (pat), HCV inhibitor tripeptides (pat), diaryl peptides such as NS3 serine protease inhibitors of HCV (pat), Imidazolidindiones as NS3 serine protease inhibitors of HCV (pat).

Thiazolidines and benzanilides (ref). Thiazolidine derivatives which show relevant inhibition in a reverse-phase HPLC assay with an NS3/4A fusion protein and NS5A/5B substrate especially compound RD-16250 possessing a fused cinnamoyl moiety substituted with a long alkyl chain, RD4 6205 and RD4 6193

Phenan-threnequinone possessing activity against protease in a SDS-PAGE and autoradiography assay isolated from the fermentation culture broth of Streptomyces sp, Sch68631 and Sch351633, isolated from the fungus Penicillium griseofulvum, which demonstrates activity in a scintillation proximity assay.

(7) Nucleoside or non-nucleoside inhibitors of HCV NS5B RNA-dependent RNA polymerase, such as 2′-C-methyl-3′-O-L-valine ester ribofuranosyl cytidine (Idenix) as disclosed in WO 2004/002422 A2 (incorporated herein by reference in its entirety), R803 (Rigel), JTK-003 (Japan Tabacco), HCV-086 (ViroPharma/Wyeth) and other compounds currently in preclinical development;

gliotoxin (ref) and the natural product cerulenin;

2′-fluoronucleosides;

other nucleoside analogues as disclosed in WO 02/057287 A2, WO 02/057425 A2, WO 01/90121, WO 01/92282, and U.S. Pat. No. 6,812,219, the disclosures of which are incorporated herein by reference in their entirety.

Idenix Pharmaceuticals discloses the use of branched nucleosides in the treatment of flaviviruses (including HCV) and pestiviruses in International Publication Nos. WO 01/90121 and WO 01/92282 (incorporated herein by reference in their entireties). Specifically, a method for the treatment of hepatitis C infection (and flaviviruses and pestiviruses) in humans and other host animals is disclosed in the Idenix publications that includes administering an effective amount of a biologically active 1′, 2′, 3′ or 4′-branced B-D or B-L nucleosides or a pharmaceutically acceptable salt or prodrug thereof, administered either alone or in combination with another antiviral agent, optionally in a pharmaceutically acceptable carrier. Certain preferred biologically active 1′, 2′, 3′, or 4′ branched B-D or B-L nucleosides, including Telbivudine, are describedi n U.S. Pat. Nos. 6,395,716 and 6,875,751, each of which are incorporated herein by reference.

Other patent applications disclosing the use of certain nucleoside analogs to treat hepatitis C virus include: PCTCA00/01316 (WO 01/32153; filed Nov. 3, 2000) and PCT/CA01/00197 (WO 01/60315; filed Feb. 19, 2001) filed by BioChem Pharma, Inc., (now Shire Biochem, Inc.); PCT/US02/01531 (WO 02/057425; filed Jan. 18, 2002) and PCT/US02/03086 (WO 02/057287; filed Jan. 18, 2002) filed by Merck & Co., Inc., PCT/EP01/09633 (WO 02/18404; published Aug. 21, 2001) filed by Roche, and PCT Publication Nos. WO 01/79246 (filed Apr. 13, 2001), WO 02/32920 (filed Oct. 18, 2001) and WO 02/48165 by Pharmasset, Ltd. (the disclosures of which are incorporated herein by reference in their entireties)

PCT Publication No. WO 99/43691 to Emory University (incorporated herein by reference in its entirety), entitled “2′-Fluoronucleosides” discloses the use of certain 2′-fluoronucleosides to treat HCV.

Eldrup et al. (Oral Session V, Hepatitis C Virus, Flaviviridae; 16^{th} International Conference on Antiviral Research (Apr. 27, 2003, Savannah, Ga.)) described the structure activity relationship of 2′-modified nucleosides for inhibition of HCV.

Bhat et al. (Oral Session V, Hepatitis C Virus, Flaviviridae, 2003 (Oral Session V, Hepatitis C Virus, Flaviviridae; 16^{th} International conference on Antiviral Research (Apr. 27, 2003, Savannah, Ga.); p A75) describes the synthesis and pharmacokinetic properties of nucleoside analogues as possible inhibitors of HCV RNA replication. The authors report that 2′-modified nucleosides demonstrate potent inhibitory activity in cell-based replicon assays.

Olsen et al. (Oral Session V, Hepatitis C Virus, Flaviviridae; 16^{th} International Conference on Antiviral Research (Apr. 27, 2003, Savannah, Ga.) p A76) also described the effects of the 2′-modified nucleosides on HCV RNA replication.

(8) Nucleotide polymerase inhibitors and gliotoxin (Ferrari R. et al. Journal of Virology, 1999, 73, 1649-1654), and the natural product cerulenin (Lohmann V. et al. Virology, 1998, 249, 108-118);

(9) HCV NS3 helicase inhibitors, such as VP_{—}50406 by ViroPhama and compounds from Vertex. Other helicase inhibitors (Diana G. D. et al., Compounds, compositions and methods for treatment of hepatitis C, U.S. Pat. No. 5,633,358 (incorporated herein by reference in its entirety); Diana G. D. et al., Piperidine derivatives, pharmaceutical compositions thereof and their use in the treatment of hepatitis C, PCT WO 97/36554);

(10) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 199, 181, 251-257); such as ISIS 14803 by Isis Pharm/Elan, antisense by Hybridon, antisense by AVI bioPharma,

(11) Inhibitors of IRES-dependent translation (Ikeda N et al., Agent for the prevention and treatment of hepatitis C, Japanese Patent Pub. JP-08268890; Kai Y et al. Prevention and treatment of viral diseases, Japanese Patent Pub. JP-10101591); such as ISIS 14803 by Isis Pharm/Elan, IRES inhibitor by Anadys, IRES inhibitors by Immusol, targeted RNA chemistry by PTC Therapeutics

(12) Ribozymes, such as nuclease-resistant ribozymes (Maccjak, D. J. et al., Hepatology 1999, 30, abstract 995) and those directed in U.S. Pat. No. 6,043,077 to Barber et al., and U.S. Pat. Nos. 5,869,253 and 5,610,054 to Draper et al. (incorporated herein by reference in their entireties) for example, HEPTAZYME by RPI

(13) siRNA directed against HCV genome

(14) HCV replication inhibitor of any other mechanisms such as by VP50406ViroPharama/Wyeth, inhibitors from Achillion, Arrow

(15) An inhibitor of other targets in the HCV life cycle including viral entry, assembly and maturation

(16) An immune modulating agent such as an IMPDH inhibitor, mycophenolic acid, a salt or a prodrug thereof sodium mycophenolate or mycophenolate mofetil, or Merimebodib (VX-497); thymosin alpha-1 (Zadaxin, by SciClone); or a S1P receptor agonist, e.g. FTY720 or analogue thereof optionally phosphorylated.

(17) An anti-fibrotic agent, such as a N-phenyl-2-pyrimidine-amine derivative, imatinib (Gleevac), IP-501 by Indevus, and Interferon gamma 1b from InterMune

(18) Therapeutic vaccine by Intercell, Epimmune/Genecor, Merix, Tripep (Chron-VacC), immunotherapy (Therapore) by Avant, T cell therapy by CellExSys, monoclonal antibody XTL-002 by STL, ANA 246 and ANA 246 BY Anadys,

(19) Other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antitoxidants (U.S. Pat. No. 5,922,757 to Chojkier et al.), amantadine, bile acids (U.S. Pat. No. 5,846,99964 to Ozeki et al.), N-(phosphonoacetl)-L-aspartic acid, (U.S. Pat. No. 5,830,905 to Diana et al.), benzenedicarboxamides (U.S. Pat. No. 5,633,388 to Diane et al.), polyadenylic acid derivatives (U.S. Pat. No. 5,496,546 to Wang et al.), 2′3′-dideoxyinosine (U.S. Pat. No. 5,026,687 to Yarchoan et al.), benzimidazoles (U.S. Pat. No. 5,891,874 to Colacino et al.), plant extracts (U.S. Pat. No. 5,837,257 to Tsai et al., U.S. Pat. No. 5,725,859 to Omer et al., and U.S. Pat. No. 6,056,961) and piperidines (U.S. Pat. No. 5,830,905 to Diana et al.); the disclosures of which are incorporated herein by reference in their entireties. Also, squalene, telbivudine, N-(phosphonoacetyl)-L-aspartic acid, benzenedicarboxamides, polyadenylic acid derivatives, glycosylation inhibitors, and nonspecific cytoprotective agents that block cell injury caused by the virus infection.

(20) Any other compound currently in preclinical or clinical development for the treatment of HCV, including Interleukin-10 (Schering-Plough), AMANTADINE (Symmetrel) by Endo Labs Solvay, caspase inhibitor IDN-6556 by Idun Pharma, HCV/MF59 by Chiron, CIVACIR (Hepatitis C Immune Globulin) by NABI, CEPLENE (histamine dichloride) by Maxim, IDN-6556 by Idun PHARM, T67, a beta-tubulin inhibitor, by Tularik, a therapeutic vaccine directed to E2 by Innogenetics, FK788 by Fujisawa Helathcare, IdB1016 (Siliphos, oral silybin-phosphatidyl choline phytosome), fusion inhibitor by Trimeris, Dication by Immtech, hemopurifier by Aethlon Medical, UT 231B by United Therapeutics.

(21) Purine nucleoside analog antagonists of TlR7 (toll-like receptors) developed by Anadys, e.g., Isotorabine (ANA245) and its prodrug (ANA975), which are described in European applications EP348446 and EP636372, International Publications WO03/045968, WO05/121162 and WO05/25583, and U.S. Pat. No. 6/973322, each of which is incorporated by reference.

(21) Non-nucleoside inhibitors developed by Genelabs and described in International Publications WO2004/108687 WO2005/12288 and WO2006/076529 each of which is incorporated by reference.

(22) Other co-agents (e.g., non-immunomodulatory or immunomodulatory compounds) that may be used in combination with a compound of this invention include, but are not limited to, those specified in WO 02/18369, which is incorporated herein by reference.

Methods of this invention may also involve administration of another component comprising an additional agent selected from an immunomodulatory agent; an antiviral agent; an inhibitor of HCV protease; an inhibitor of another target in the HCV life cycle; a CYP inhibitor; or combinations thereof.

Accordingly, in another embodiment, this invention provides a method comprising administering a compound of the invention and another anti-viral agent, preferably an anti-HCV agent. Such anti-viral agents include, but are not limited to, immunomodulatory agents, such as α, β, and δ interferons, pegylated derivatized interferon-a compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds of U.S. Pat. Nos. 5,807,876, 6,498,178, 6,344,465, 6,054,472, WO 97/40028, WO 98/40381, WO 00/56331, and mycophenolic acid and derivatives thereof, and including, but not limited to VX-497, VX-148, and/or VX-944); or combinations of any of the above.

In accordance with the foregoing the present invention provides in a yet further aspect:
A pharmaceutical combination comprising a) a first agent which is a compound of the invention, e.g. a compound of formula I or any subformulae thereof, and b) a co-agent, e.g. a second drug agent as defined above.
A method as defined above comprising co-administration, e.g. concomitantly or in sequence, of a therapeutically effective amount of a compound of the invention, e.g. a compound of formula I or any subformulae thereof, and a co-agent, e.g. a second drug agent as defined above.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time. Fixed combinations are also within the scope of the present invention. The administration of a pharmaceutical combination of the invention results in a beneficial effect, e.g. a synergistic therapeutic effect, compared to a monotherapy applying only one of its pharmaceutically active ingredients.

Each component of a combination according to this invention may be administered separately, together, or in any combination thereof. As recognized by skilled practitioners, dosages of interferon are typically measured in IU (e.g., about 4 million IU to about 12 million IU).

If an additional agent is selected from another CYP inhibitor, the method would, therefore, employ two or more CYP inhibitors. Each component may be administered in one or more dosage forms. Each dosage form may be administered to the patient in any order.

The compound of the invention and any additional agent may be formulated in separate dosage forms. Alternatively, to decrease the number of dosage forms administered to a patient, the compound of the invention and any additional agent may be formulated together in any combination. For example, the compound of the invention inhibitor may be formulated in one dosage form and the additional agent may be formulated together in another dosage form. Any separate dosage forms may be administered at the same time or different times.

Alternatively, a composition of this invention comprises an additional agent as described herein. Each component may be present in individual compositions, combination compositions, or in a single composition.

### Exemplifcation of the Invention

The invention is further illustrated by the following examples, which should not be construed as further limiting. The assays used throughout the Examples are accepted. Demonstration of efficacy in these assays is predictive of efficacy in subjects.

### General Synthesis Methods

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

HPLC (Method A):

Instrument: Agilent system
column: Macherey-Nagel Nucleosil 100-3 C18 HD, particle size 3.5 μm, pore size 100 Å, length 70 mm, internal diameter 4 mm, flow 1.0 ml/min
solvent: CH₃CN (0.1% CF₃CO₂H); H₂O (0.1% CF₃CO₂H)
gradient: 0-6 min: 20-100% CH₃CN, 1.5 min: 100% CH₃CN, 0.5 min 100-20% CH₃CN
   HPLC (Method B):
Instrument: Agilent system
column: waters symmetry C18, 3.5 μm, 2.1×50 mm, flow 0.6 ml/min
solvent: CH₃CN (0.1% CF₃CO₂H); H₂O (0.1% CF₃CO₂H)
gradient: 0-3.5 min: 20-95% CH₃CN, 3.5-5 min: 95% CH₃CN, 5.5-5.55 min 95% to 20% CH₃CN
   Preparative HPLC (Method C):
Instrument: Gilson
Column: Sun-Fire prep C18 OBD 5 μm, Column 19×50 mm (flow 20 mL/min) or
Column 30×100 mm (flow 40 mL/min)
Solvent: CH₃CN (0.1% CF₃CO₂H) and H₂O (0.1% CF₃CO₂H)
Gradient: 0-20 min: 5-100% CH₃CN
   Preparative HPLC (Method D):
Instrument: Gilson system
column: waters C18 ODB, 5 μm, 50×19 mm
solvent: CH₃CN (0.1% HCO₂H); H₂O (0.1% HCO₂H)
   MS (Method E):
Instrument: Agilent 1100 Series
Detection: API-ES, positive/negative
LC-MS (method F):
Instrument: Agilent system
Column: Waters symmetry, 3.5 μm, 50×2.1 mm, 5 min, 20% to 95% CH₃CN
solvent: CH₃CN (0.1% HCO₂H); H₂O (0.1% HCO₂H)
gradient: 0-3.5 min: 20-95% CH₃CN, 3.5-5 min: 95% CH₃CN, 5.5-5.55 min 95% to 20% CH₃CN

### EXAMPLE 1

### {[(1R,2S)-1-(3-Benzyloxy-benzenesulfonylaminocarbonyl)-2-vinyl-cyclopropylcarbamoyl]-methyl}-cyclopentylmethyl-carbamic acid tert-butyl ester

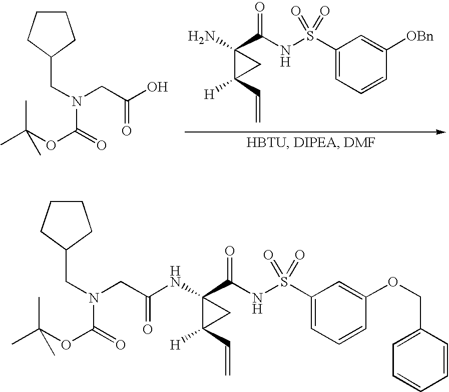

To a 10 mL round bottom flask containing (tert-Butoxycarbonyl-cyclopentylmethyl-amino)-acetic acid (38 mg, 0.15 mmol), N-((1R,2S)-1-Amino-2-vinyl-cyclopropanecarbonyl)-3-benzyloxy-benzenesulfonamide (61 mg, 0.15 mmol) and DIPEA (0.13 mL, 0.74 mmol) in DMF (3 mL) is added at 0° C. HBTU (68 mg, 0.18 mmol). After stirring overnight at RT, the reaction mixture is directly purified by preparative RP HPLC (method C) to furmish the product (45 mg, 0.07 mmol).

HPLC (method A) t_{R}=5.89

MS (method E)=610 [M−H]⁺

TLC (CH₂Cl₂/MeOH: 19:1) Rf=0.43

### Preparation of tert-Butoxycarbonyl-cyclopentylmethyl-amino)-acetic acid

### Step 1-1

### (Cyclopentylmethyl-amino)-acetic acid methyl ester

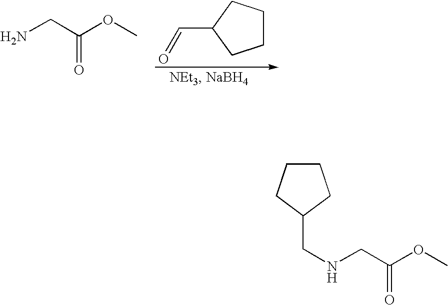

To a 500 mL round bottom flask containing MeOH (250 mL) and 2 g molecular sieves (4 Å) is added Cyclopentanecarboxaldehyde (9 g, 89 mmol), Glycine-methylester (HCl-salt) (11.3 g, 89 mmol) and NEt₃ (18 mL, 116 mmol). After 30 min NaBH₄ (4.5 g, 116 mmol) is added at 0° C. in 5 portions. After stirring for 2 h at RT the reactions is quenched by addition of NaHCO₃ (Sat'd, 50 mL), sat. bicarbonate. The solvent is removed in vacuo dissolved in water (100 mL) and extracted with CH₂Cl₂ (3×100 mL). The organic phase is dried with Na₂SO₄, filtered and the solvent is removed in vacuo. The residue is purified by FCC (Hexane/EtOAc 1:1) to furnish the product (5.9 g, 34 mmol).

MS (method E)=172 [M+H]⁺

TLC (Hexane/EtOAc: 1:1) Rf=0.55

### Step 1-2

### (tert-Butoxycarbonyl-cyclopentylmethyl-amino)-acetic acid methyl ester

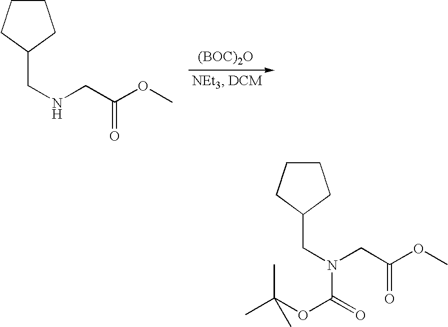

To a 250 mL round bottom flask containing (Cyclopentylmethyl-amino)-acetic acid methyl ester (1 g, 6.2 mmol) in CH₂Cl₂ (60 mL) is added at 0° C. NEt₃ (1.7 mL, 12.4 mmol) followed by (BOC)2O (2.0 g, 9.3 mmol). After 15 min the mixture is warmed to RT and stirred for 2 h. The reaction is quenched by addition of NaHCO₃ (Sat'd, 50 mL), extracted with CH₂Cl₂ (3×50 mL), dried with Na₂SO₄, filtered and the solvent is removed in vacuo. The residue is purified by FCC (Hexane/EtOAc 1:1) to furnish the product (1.3 g, 4.8 mmol).

MS (method E)=216 [M−55]⁺

TLC (Hexane/EtOAc: 1:1) Rf=0.86

### Step 1-3

### (tert-Butoxycarbonyl-cyclopentylmethyl-amino)-acetic acid

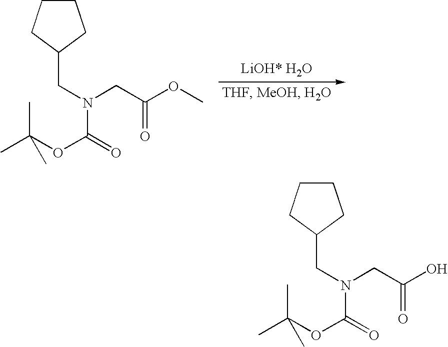

To a 50 mL round bottom flask containing (tert-Butoxycarbonyl-cyclopentylmethyl-amino)-acetic acid methyl ester (1.22 g, 4.5 mmol) in 40 mL THF/MeOH/H₂O (2:1:1) is added LiOH (0.56 g, 13.5 mmol) at RT and the mixture is stirred overnight. The solvent is removed in vacuo, the residue is acidified with 4 N HCl, extracted with EtOAc (3×50 mL), washed with brine, dried with Na₂SO₄, filtered and the solvent is removed in vacuo. The residue is purified by FCC (CH₂Cl₂/MeOH: 19:1) to furnish the product (1.20 g, 4.5 mmol).

MS (method E)=256 [M−H]⁺

TLC (CH₂Cl₂/MeOH: 19:1) Rf=0.34

### Preparation of N-((1R,2S)-1-Amino-2-vinyl-cyclopropanecarbonyl)-3-benzyloxy-benzenesulfonamide

### Step 1-4

### 1-Benzyloxy-3-bromo-benzene

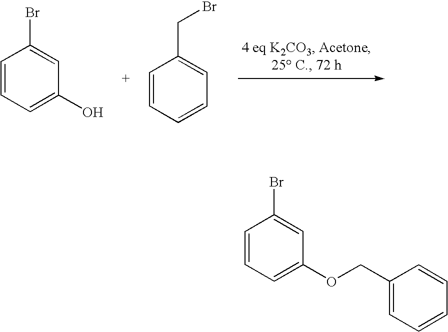

3-Bromophenol (19 g) and benzylbromide (15.7 mL) in acetone (200 mL) are treated with potassium carbonate (60.1 g) and the reaction mixture is stirred at RT for 72 hours. The reaction is filtered and the filter cake is washed with acetone. The filtrate is concentrated and purified via chromatography on SiO₂ gel (eluent hexanes/EtOAc 96:4) to give 1-benzyloxy-3-bromobenzene as a white solid.

### Step 1-5

### 3-Benzyloxy-benzenesulfonamide

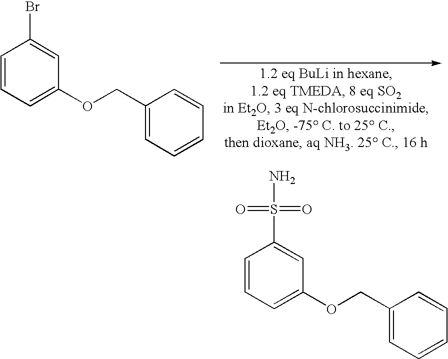

A solution of 1-benzyloxy-3-bromobenzene (28.3 g) in Et₂O (375 mL) is cooled to −70° C. and treated with TMEDA (19.2 mL) and n-BuLi in hexane (1.6 M, 79 mL). The solution is stirred at −70° C. for 1 h and transferred into a cooled solution (−70° C.) of SO₂ (54.4 g) in Et₂O (375 mL). The mixture is kept at −70° C. for 15 minutes, then allowed to warm to RT over 1 h. The solvent is evaporated and the residue is suspended in aqueous sodium phosphate (1M, 750 mL, pH 6). EtOAc (500 mL) is added and the solution is cooled to 0° C. N-Chlorosuccinimide (43.5 g) is slowly added and the pH is readjusted to pH 6 by addition of Na₃PO₄. The reaction mixture is stirred vigorously for 1 h. The phases are separated and the aqueous phase is extracted twice with EtOAc. The combined organic phases are washed with H₂O and brine, dried and concentrated to give a yellowish oil. The residue is taken up in dioxane (400 mL) and NH₃ in H₂O (28%, 200 mL) is added. The reaction mixture is stirred for 12 h and then concentrated to dryness. The residue is chromatographed on SiO₂ gel (eluent hexanes/EtOAc 4:1 to 3:7) to give 3-benzyloxy-benzenesulfonamide as a white powder.

API-MS: M−1=262.

### Step 1-6

### [(1R,2S)-1-(3-Benzyloxy-benzenesulfonylaminocarbonyl)-2-vinyl-cyclopropyl]-carbamic acid tert-butyl ester

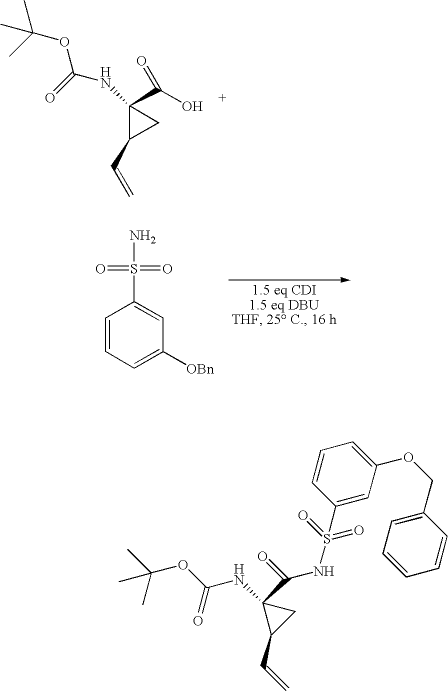

A solution of 0.7 g of (1R,2S)-1-tert-butoxycarbonylamino-2-vinyl-cyclopropane-carboxylic acid (prepared as described in Journal of Organic Chemistry, 2005, 5869-5879) in THF (10 mL) is treated with carbonyldiimidazole (0.789 g) and the reaction mixture is stirred at 65° C. for 30 min. The mixture is allowed to cool to RT and 3-benzyloxy-benzenesulfonamide (1.05 g) and DBU (0.697 ml) are added. The solution is stirred at RT for 12 h. The reaction mixture is taken up in EtOAc, washed with 0.1N aqueous HCl, aqueous NaHCO₃ and brine, dried with Na₂SO₄ and concentrated. The residue is chromatographed on SiO₂ gel (eluent hexanes/EtOAc 7:3 to EtOAc, then EtOAc/MeOH 9:1) to give [(1R,2 )-1-(3-benzyloxy-benzenesulfonylaminocarbonyl)-2-vinyl-cyclopropyl]-carbamic acid tert-butyl ester.

API-MS: M+1=473.

### Step 1-7

### N-((1R,2S)-1-Amino-2-vinyl-cyclopropanecarbonyl)-3-benzyloxy-benzenesulfonamide

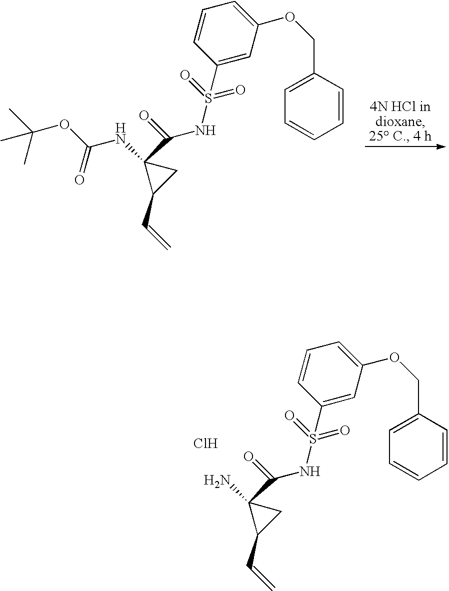

A solution of [(1R,2S)-1-(3-benzyloxy-benzenesulfonylaminocarbonyl)-2-vinyl-cyclopropyl]-carbamic acid tert-butyl ester (0.85 g) in dioxane (5 mL) is treated with HCl in dioxane (4N, 10 mL) and is stirred at RT for 4 h. The reaction mixture is evaporated to give N-((1R, 2S)-1-amino-2-vinyl-cyclopropanecarbonyl)-3-benzyloxy-benzenesulfonamide hydrochloride.

API-MS: M+1=373.

### EXAMPLE 2

### [(S)-1-({[(1R,2S)-1-(3-Benzyloxy-benzenesulfonylaminocarbonyl)-2-vinyl-cyclopropylcarbamoyl]-methyl-cyclopentylmethyl-carbamoyl)-2-methyl-propyl]-carbamic acid tert-butyl ester

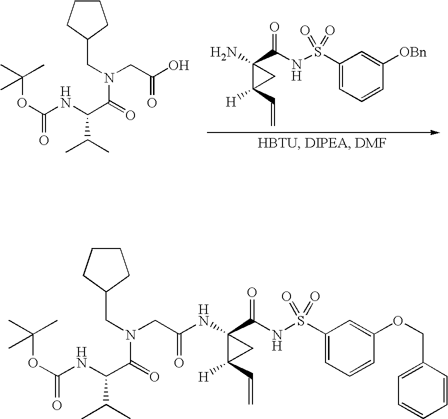

To a 10 mL round bottom flask containing [((S)-2-tert-Butoxycarbonylamino-3-methyl-butyryl)-cyclopentylmethyl-amino]-acetic acid (54 mg, 0.15 mmol), N-((1R,2 S)-1-Amino-2-vinyl-cyclopropanecarbonyl)-3-benzyloxy-benzenesulfonamide (50 mg, 0.12 mmol) and DIPEA (0.10 mL, 0.61 mmol) in DMF (2 mL) is added at 0° C. HBTU (55 mg, 0.15 mmol). After stirring overnight at RT, the reaction mixture is directly purified by preparative RP HPLC (method C) to furnish the product (59 mg, 0.08 mmol).

HPLC (method A) t_{R}=6.06

MS (method E)=709 [M−H]⁺

TLC (CH₂Cl₂/MeOH: 19:1) Rf=0.49

### Preparation of [((S)-2-tert-Butoxycarbonylamino-3-methyl-butyryl)-cyclopentylmethyl-amino]-acetic acid

### Step 2-1

### [((S)-2-tert-Butoxycarbonylamino-3-methyl-butyryl)-cyclopentylmethyl-amino]-acetic acid methyl ester

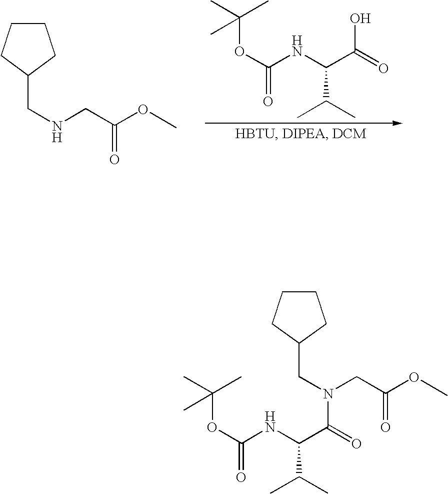

To a 250 mL round bottom flask containing (Cyclopentylmethyl-amino)-acetic acid methyl ester (1 g, 6.2 mmol) in CH₂Cl₂ (60 mL) is added at RT N-BOC-L-Valine (1.3 g, 5.8 mmol) and DIPEA, (4.0 mL, 23.4 mmol). The mixture is cooled to 0° C. and HBTU (2.8 g, 5.8 mmol) is added. After 60 min the mixture is warmed to RT and stirred overnight. The reaction is quenched by addition of NaHCO₃ (Sat'd, 50 mL), washed with water (2×30 mL), dried with Na₂SO₄, filtered and the solvent is removed in vacuo. The residue is purified by FCC (Hexane/EtOAc 9:1) to furnish the product (1.93 g, 5.2 mmol).

MS (method E)=371 [M+H]⁺

TLC (Hexane/EtOAc: 1:1) Rf=0.66

### Step 2-2

### [((S)-2-tert-Butoxycarbonylamino-3-methyl-butyryl)-cyclopentylmethyl-amino]-acetic acid

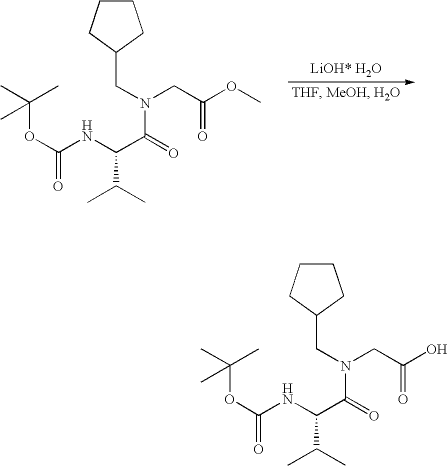

To a 50 mL round bottom flask containing (tert-Butoxycarbonyl-cyclopentylmethyl-amino)-acetic acid methyl ester (1.9 g, 5.2 mmol) in 40 mL THF/MeOH/H₂O (2:1:1) is added LiOH (0.66 g, 15.6 mmol) at RT and the mixture is stirred overnight. The solvent is removed in vacuo, the residue is acidified with 4 N HCl, extracted with EtOAc (3×50 mL), washed with brine, dried with Na₂SO₄, filtered and the solvent is removed in vacuo. The residue is purified by FCC (CH₂Cl₂/MeOH: 19:1) to furnish the product (1.60 g, 4.5 mmol).

MS (method E)=357 [M+H]⁺

TLC (CH₂Cl₂/MeOH: 19:1) Rf=0.30

### EXAMPLE 3

### [(S)-1-(Cyclopentylmethyl-{[(1R,2S)-1-(2-methylamino-benzenesulfonylaminocarbonyl)-2-vinyl-cyclopropylcarbamoyl]-methyl}-carbamoyl)-2-methyl-propyl]-carbamic acid tert-butyl ester

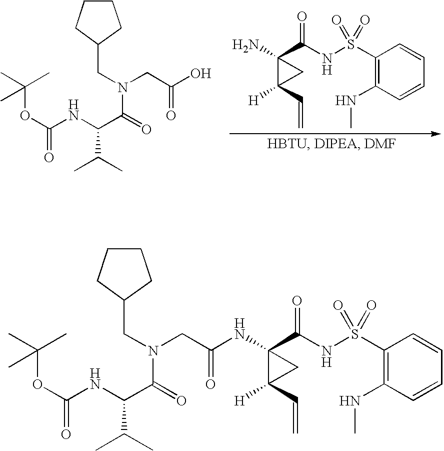

To a 10 mL round bottom flask containing [((S)-2-tert-Butoxycarbonylamino-3-methyl-butyryl)-cyclopentylmethyl-amino]-acetic acid (75 mg, 0.20 mmol), N-((1R,2S)-1-Amino-2-vinyl-cyclopropanecarbonyl)-2-methylamino-benzenesulfonamide (50 mg, 0.17 mmol) and DIPEA (0.15 mL, 0.95 mmol) in DMF (2 mL) is added at 0° C. HBTU (77 mg, 0.20 mmol). After stirring overnight at RT, the reaction mixture is directly purified by preparative RP HPLC (method C) to furnish the product (53 mg, 0.10 mmol).

HPLC (method A) t_{R}=5.59 min

MS (method E)=634 [M+H]⁺

TLC (CH₂Cl₂/MeOH: 19:1) Rf=0.52

### Preparation of N-((1R,2S)-1-Amino-2-vinyl-cyclopropanecarbonyl)-2-methylamino-benzenesulfonamide

### Step 3-1

### [(1R,2S)-1-(2-Amino-benzenesulfonylaminocarbonyl)-2-vinyl-cyclopropyl]-carbamic acid tert-butyl ester

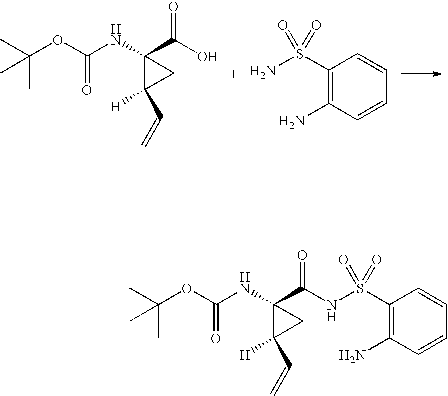

To a solution of 6.3 g (28 mmol) (1R,2S)-1-tert-Butoxycarbonylamino-2-vinyl-cyclopropane-carboxylic acid (prepared according to WO 2000009558 A1) in 90 mL abs. THF is added 6.95 g (42 mmol) CDI and the mixture is refluxed for 2 h. After cooling to rt 5.1 g (29 mmol) 2-Aminobenzenesulfonamide and 6.5 g (42 mmol) DBU is added and stirring is continued for 45 min. The reaction mixture is diluted with 250 mL EtOAc and washed with 100 mL 0.5 N HCl and brine. The organic phase is dried with Na₂SO₄, filtered and the solvent is removed in vacuo. The residue is purified by FC on silica (eluent: CH₂Cl₂/MeOH 98:2) to give the title compound as a colorless solid.

HPLC (method A) t_{R}=3.99 min

MS (method E)=382 [M+H]⁺

TLC, Rf (CH₂Cl₂/MeOH 19:1)=0.35

### Step 3-2

### [(1R,2S)-1-(2-Methylamino-benzenesulfonylaminocarbonyl)-2-vinyl-cyclopropyl]-carbamic acid tert-butyl ester

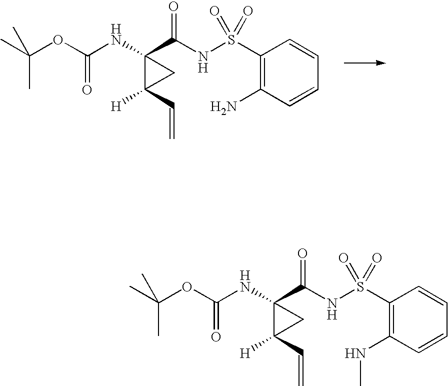

Methyl iodide (0.18 mL, 2.83 mmol) is added to amixture of [(1R,2S)-1-(2-Amino-benzenesulfonylaminocarbonyl)-2-vinyl-cyclopropyl]-carbamic acid tert-butyl ester (1.08 g, 2.83 mmol) and K₂CO₃ (435 mg, 3.11 mmol) in DMF (30 mL). After stirring for 1 hour, the reaction mixture is concentrated in vacuo and the residue is chromatographed by preparative reverse phase HPLC (Method D) to give [(1R,2S)-1-(2-Methylamino-benzenesulfonylamino-carbonyl)-2-vinyl-cyclopropyl]-carbamic acid tert-butyl ester as a white solid.

LC-MS (method F) t_{R}=4.03; [M+H]=396.0

### N-((1R,2S)-1-Amino-2-vinyl-cyclopropanecarbonyl)-2-methylamino-benzene sulfonamide hydrochloride

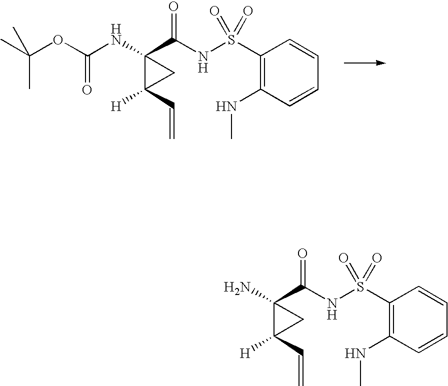

A mixture of [(1R,2S)-1-(2-Methylamino-benzenesulfonyl-aminocarbonyl)-2-vinyl-cyclopropyl]-carbamic acid tert-butyl ester (558 mg, 1.41 mmol) in 3.5 ml HCl (4M in dioxane) and 3.5 ml dioxane is stirred at room temperature for 2 hours. Evaporation of the solvent affords N-((1R,2S)-1-Amino-2-vinyl-cyclopropanecarbonyl)-2-methylamino-benzene sulfonamide hydrochloride as a yellowish solid.

HPLC (method B) t_{R}=0.95 min

LC-MS (method F) t_{R}=0.87; [M+H]=296.0

### EXAMPLE 4

### [(S)-1-(Cyclopentylmethyl-{[(1R,2S)-1-(2-methylamino-benzenesulfonylaminocarbonyl)-2-vinyl-cyclopropylcarbamoyl]-methyl}-carbamoyl)-2-methyl-propyl]-carbamic acid tert-butyl ester

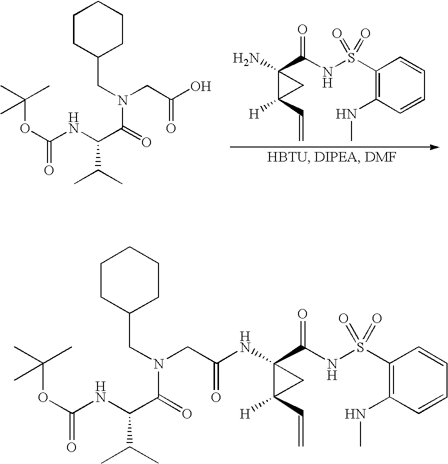

The title compound is prepared analogously as described in Example 3 using [((S)-2-tert-Butoxycarbonylamino-3-methyl-butyryl)-cyclohexylmethyl-amino]-acetic acid (75 mg, 0.20 mmol), N-((1R,2S)-1-Amino-2-vinyl-cyclopropanecarbonyl)-2-methylamino-benzene-sulfonamide (50 mg, 0.17 mmol), DIPEA (0.15 mL, 0.85 mmol) and HBTU (77 mg, 0.20 mmol) in DMF (2 mL).

HPLC (method A) t_{R}=5.86 min

MS (method E)=648 [M+H]⁺

TLC (CH₂Cl₂/MeOH: 1:1) Rf=0.49

### Step 4-1

### (Cyclohexylmethyl-amino)-acetic acid methyl ester

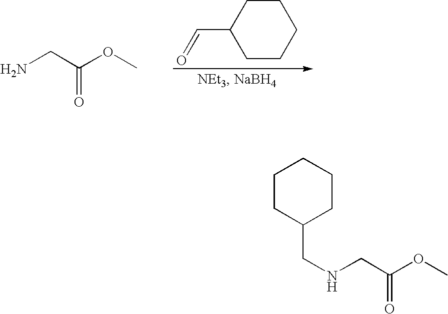

The title compound is prepared analogously as described in Example 1 (step 1) using Cyclohexanecarboxaldehyde (11.2 g, 100 mmol), Glycine-methylester (HCl-salt) (12.5 g, 100 mmol), NEt₃ (18 mL, 130 mmol) and NaBH₄ (5.2 g, 130 mmol) in MeOH (300 mL).

MS (method E)=186 [M+H]⁺

TLC (Hexane/EtOAc: 1:1) Rf=0.37

### Step 4-2

### [((S)-2-tert-Butoxycarbonylamino-3-methyl-butyryl)-cyclohexylmethyl-amino]-acetic acid methyl ester

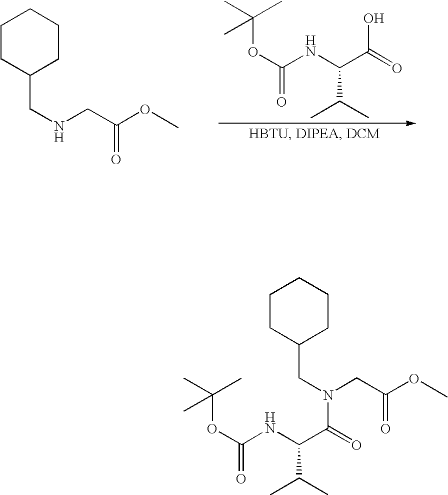

The title compound is prepared analogously as described in Example 2 (step 1) using (Cyclohexylmethyl-amino)-acetic acid methyl ester (1.85, 10 mmol), N-BOC-L-Valine (2.2 g, 10 mmol), DIPEA (6.8 mL, 40 mmol) and HBTU (4.7 g, 12.5 mmol) in in CH₂Cl₂ (100 mL).

MS (method E)=385 [M+H]⁺

TLC (Hexane/EtOAc: 1:1) Rf=0.80

### Step 4-3

### [((S)-2-tert-Butoxycarbonylamino-3-methyl-butyryl)-cyclohexylmethyl-amino]-acetic acid

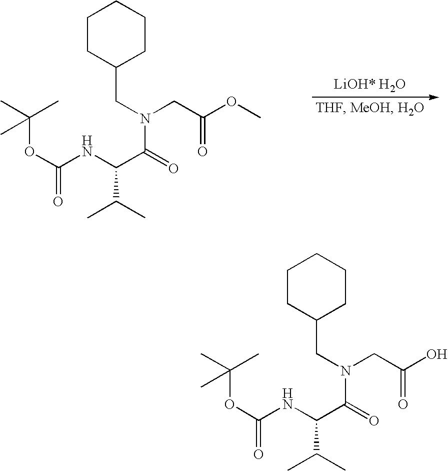

The title compound is prepared analogously as described in Example 2 (step 2) using [((S)-2-tert-Butoxycarbonylamino-3-methyl-butyryl)-cyclohexylmethyl-amino]-acetic acid methyl ester (3.61 g, 9.4 mmol) and LiOH (1.2 g, 28 mmol) in 100 mL THF/MeOH/H₂O (2:1:1).

MS (method E)=370 [M−H]⁺

TLC (Hexane/EtOAc: 1:1) Rf=0.26

### EXAMPLE 5

### [(S)-1-(Cyclopentylmethyl-{[(1R,2S)-1-(2-methylamino-benzenesulfonylaminocarbonyl)-2-vinyl-cyclopropylcarbamoyl]-methyl}-carbamoyl)-2-methyl-propyl]-carbamic acid tert-butyl ester

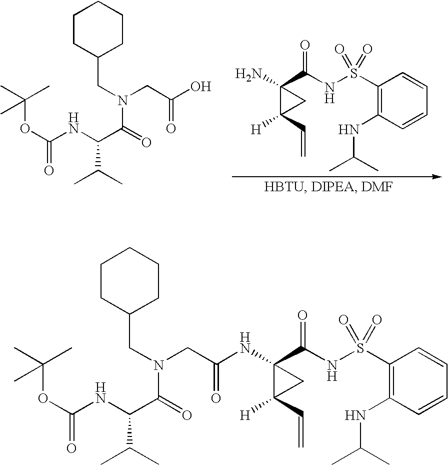

The title compound is prepared analogously as described in Example 3 using [((S)-2-tert-Butoxycarbonylamino-3-methyl-butyryl)-cyclohexylmethyl-amino]-acetic acid (74 mg, 0.20 mmol), N-((1R,2S)-1-Amino-2-vinyl-cyclopropanecarbonyl)-2-isopropylamino-benzenesulfonamide (HCl-salt) (79 mg, 0.17 mmol), DIPEA (0.10 mL, 0.6 mmol) and HATU (114 mg, 0.30 mmol) in DCM (10 mL).

HPLC (method A) t_{R}=6.22 min

MS (method E)=676 [M]⁺

TLC (DCM/MeOH: 19:1) Rf=0.20

### Step 5-1

### 2-Isopropylamino-benzenesulfonamide

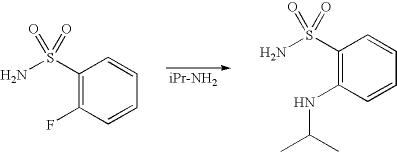

A 10 mL-microwave vial is charged with 2-Fluorobenzenesulfonamide (1.1 g, 6.3 mmol) and iso-propylamine (1.8 g, 31.4 mmol). The vial is sealed and heated for 3 h at 130° C. in a microwave (PersonalChemistry, Emrys Optimizer). The solvent is removed in vacuo and the residue is purified by FCC (DCM/MeOH 98:2->95:5) to furnish the product (1.1 g, 5.1 mmol).

HPLC (method A) t_{R}=3.24 min

MS (method E)=215 [M+H]⁺

TLC (DCM/MeOH: 19:1) Rf=0.49

### Step 5-2

### [(1R,2S)-1-(2-Isopropylamino-benzenesulfonylaminocarbonyl)-2-vinyl-cyclopropyl]-carbamic acid tert-butyl ester

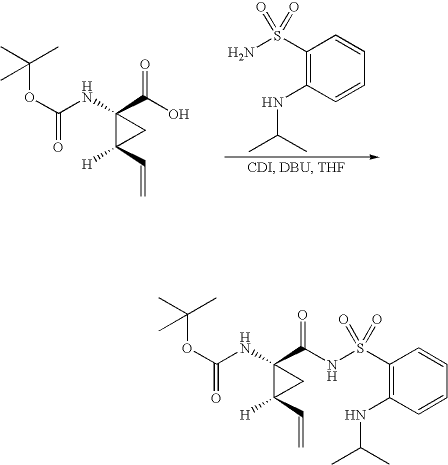

A solution of (1R,2S)-1-tert-Butoxycarbonylamino-2-vinyl-cyclopropanecarboxylic acid (0.68 g, 3.0 mmol) and CDI (0.73 g, 4.5 mmol) in THF (20 mL) is refluxed for 2 h. After cooling to ambient temperature 2-Isopropylamino-benzenesulfonamide (0.67 g, 3.1 mmol) and DBU (0.68 g, 4.5 mmol) are added and stirring is continued at RT overnight. The reaction mixture is diluted with EtOAc (50 mL) and washed with 0.5 N aq. HCl (30 mL) and brine (30 mL). The solvent is removed in vacuo and the residue is purified by FCC (DCM/MeOH 98:2→95:5) to furnish the product (0.85 g, 2.0 mmol).

HPLC (method A) t_{R}=5.01 min

MS (method E)=424 [M+H]⁺

TLC (DCM/MeOH: 19:1) Rf=0.45

### Step 5-3

### N-((1R,2S)-1-Amino-2-vinyl-cyclopropanecarbonyl)-2-isopropylamino-benzenesulfonamide

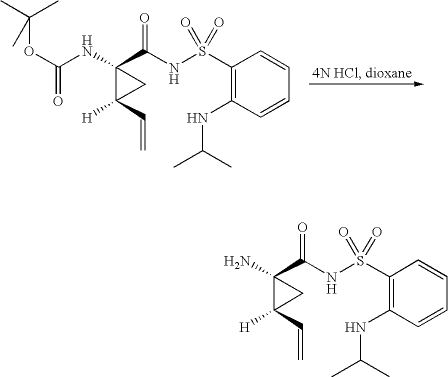

To a solution of [(1R,2S)-1-(2-Isopropylamino-benzenesulfonylaminocarbonyl)-2-vinyl-cyclopropyl]-carbamic acid tert-butyl ester (0.80 g, 1.9 mmol) in 1,4-dioxane (2 mL) is added HCl (4N in 1,4-dioxane, 4 mL). After stirring overnight at RT the solvent is removed in vacuo and the residue is used without further purification.

HPLC (method A) t_{R}=1.96 min

MS (method E)=324 [M+H]⁺

### EXAMPLE 6

### [(S)-1-(Cyclohexylmethyl-{[(1S,2R)-2-(1H-indole-7-sulfonylaminocarbonyl)-bicyclopropyl-2-ylcarbamoyl]-methyl}-carbamoyl)-2-methyl-propyl]-carbamic acid tert-butyl ester

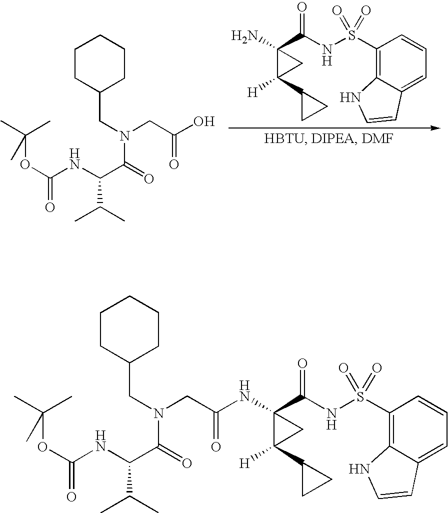

The title compound is prepared analogously as described in Example 3 using [((S)-2-tert-Butoxycarbonylamino-3-methyl-butyryl)-cyclohexylmethyl-amino]-acetic acid (80 mg, 0.22 mmol), 1H-Indole-7-sulfonic acid ((1S,2R)-2-amino-bicyclopropyl-2-carbonyl)-amide (HCl-salt) (77 mg, 0.22 mmol), DIPEA (0.11 mL, 0.65 mmol) and HATU (123 mg, 0.32 mmol) in DCM (5 mL).

HPLC (method A) t_{R}=5.94 min

MS (method E)=670 [M−H]⁺

### Step 6-1

### (1S,2R)-2-tert-Butoxycarbonylamino-bicyclopropyl-2-carboxylic acid methyl ester

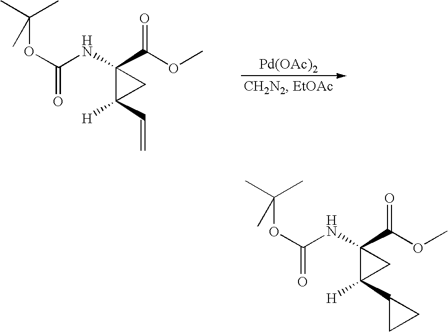

A 250 mL Erlenmeyer flask containing 40% aq. KOH (40 mL) and 40 mL Diethylether is cooled in an ice-bath. N-Nitroso-N-methylurea (1.00 g, 9.95 mmol) is added in one portion under vigorous stirring. After stirring for 15 min, the phases are separated and the diazomethane solution (40 mL, ˜0.25 M CH₂N₂ in Et₂O) is added at RT to a solution of (1R,2S)-1-tert-Butoxycarbonylamino-2-vinyl-cyclopropanecarboxylic acid methyl ester (0.48 g, 2.0 mmol) and Pd(OAc)₂ (45 mg, 0.2 mmol) in EtOAc (50 mL). After stirring overnight at RT the solvent is removed in vacuo and the residue is purified by FCC (Hexane/EtOAc 4:1) to furnish the product (0.43 g, 1.7 mmol).

HPLC (method A) t_{R}=4.10 min

MS (method E)=156 [M-BOC]⁺

TLC (Hexane/EtOAc 4:1) Rf=0.50

### Step 6-2

### (1S,2R)-2-tert-Butoxycarbonylamino-bicyclopropyl-2-carboxylic acid

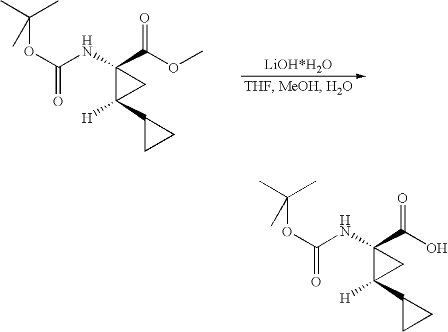

The title compound is prepared analogously as described in Example 2 (step 2) using (1S,2R)-2-tert-Butoxycarbonylamino-bicyclopropyl-2-carboxylic acid methyl ester (0.42 g, 1.6 mmol) and LiOH (98 mg, 4.1 mmol) in 10 mL THF/MeOH/H₂O (2:1:1).

MS (method E)=142 [M-BOC]⁺

TLC (DCM/MeOH 9:1) Rf=0.5

### Step 6-3

### [(1S,2R)-2-(1H-Indole-7-sulfonylaminocarbonyl)-bicyclopropyl-2-yl]-carbamic acid tert-butyl ester

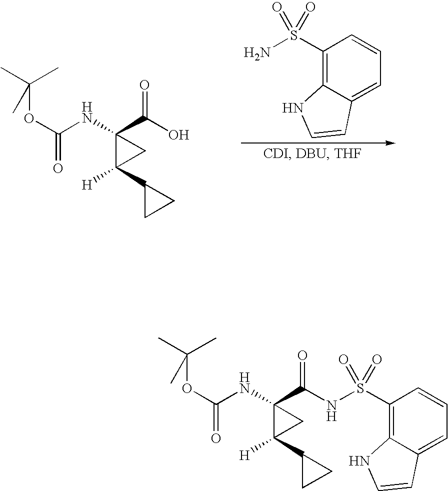

A solution of (1S,2R)-2-tert-Butoxycarbonylamino-bicyclopropyl-2-carboxylic acid (0.40 g, 1.6 mmol) and CDI (0.40 g, 2.5 mmol) in THF (20 mL) is refluxed for 2 h. After cooling to ambient temperature 1H-Indole-7-sulfonic acid amide (0.34 g, 1.7 mmol, prepared as described in U.S. Pat. No. 468,300, July 1987) and DBU (0.38 g, 2.5 mmol) are added and stirring is continued at RT overnight. The reaction mixture is diluted with EtOAc (50 mL) and washed with 0.5 N aq. HCl (30 mL) and brine (30 mL). The solvent is removed in vacuo and the residue is purified by FCC (DCM/MeOH 98:2->95:5) to furnish the product (0.47 g, 1.1 mmol).

HPLC (method A) t_{R}=4.59 min

MS (method E)=418 [M−H]⁺

TLC (DCM/MeOH: 19:1) Rf=0.37

### Step 6-4

### 1H-Indole-7-sulfonic acid((1S,2R)-2-amino-bicyclopropyl-2-carbonyl)-amide

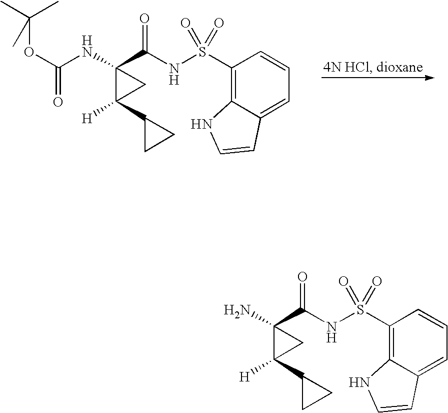

To a solution of [(1S,2R)-2-(1H-Indole-7-sulfonylaminocarbonyl)-bicyclopropyl-2-yl]-carbamic acid tert-butyl ester (0.40 g, 0.9 mmol) in 1,4-dioxane (2 mL) is added HCl (4N in 1,4-dioxane, 5 mL). After stirring overnight at RT the solvent is removed in vacuo and the residue is used without further purification.

HPLC (method A) t_{R}=2.60 min

MS (method E)=318 [M−H]⁺

### EXAMPLE 7

### [(S)-1-(Cyclopentylmethyl-{[(1R,2S)-1-(1H-indole-7-sulfonylaminocarbonyl)-2-vinyl-cyclopropylcarbamoyl]-methyl}-carbamoyl)-2-methyl-propyl]-carbamic acid tert-butyl ester

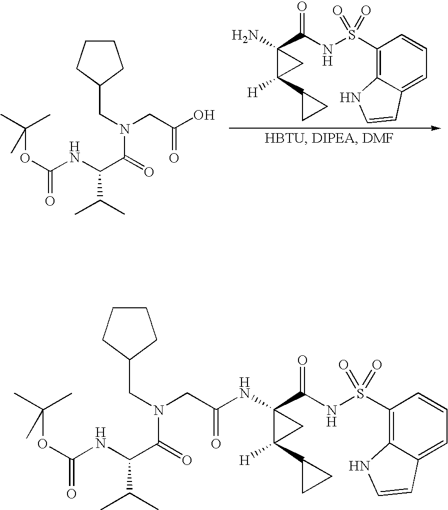

The title compound is prepared analogously as described in Example 3 using [((S)-2-tert-Butoxycarbonylamino-3-methyl-butyryl)-cyclopentylmethyl-amino]-acetic acid (73 mg, 0.16 mmol), 1H-Indole-7-sulfonic acid ((1R,2S)-1-amino-2-vinyl-cyclopropane-carbonyl)-amide (HCl-salt) (50 mg, 0.16 mmol), DIPEA (0.14 mL, 0.82 mmol) and HBTU (75 mg, 0.20 mmol) in DMF(2 mL).

HPLC (method A) t_{R}=5.69 min

MS (method E)=642 [M−H]⁺

TLC (DCM/MeOH: 19:1) Rf=0.54

### Preparation of 1H-Indole-7-sulfonic acid((1R,2S)-1-amino-2-vinyl-cyclopropane-carbonyl)-amide

### Step 7-1

### [(1R,2S)-1-(1H-Indole-7-sulfonylaminocarbonyl)-2-vinyl-cyclopropyl]-carbamic acid tert-butyl ester

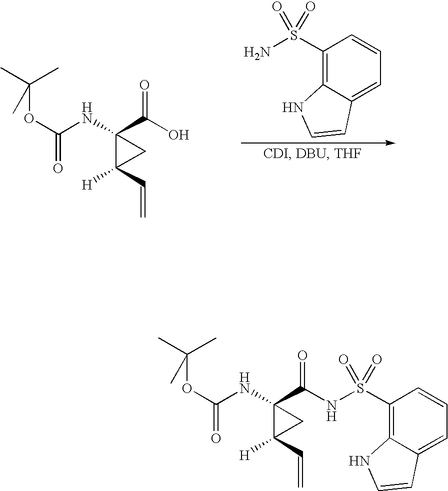

A mixture of 8.3 g (37 mmol) (1R,2S)-1-tert-Butoxycarbonylamino-2-vinyl-cyclopropane-carboxylic acid and 9.0 g (55 mmol) CDI in 200 mL THF is refluxed for 1 h, cooled to RT and 8.6 g (44 mmol) 1H-Indole-7-sulfonic acid amide (prepared as described in U.S. Pat. No. 468,300, July 1987) and 8.3 mL (55 mmol) DBU are added. The mixture is stirred at RT overnight, diluted with EtOAc and washed three times with aq. NaHCO₃-solution. The combined aq. layers are extracted with EtOAc and the combined organic layers are dried over Na₂SO₄ and concentrated under reduced pressure. The residue is purified by FCC (silica gel, eluent: DCM/MeOH 19:1) to give the title compound.

LC-MS (Method F): t_{R}=3.803, M+H=404.2

TLC (hexane/EtOAc: 1:1): Rf=0.52

### Step 7-2

### [(1R,2S)-1-(1H-Indole-7-sulfonylaminocarbonyl)-2-vinyl-cyclopropyl]-carbamic acid tert-butyl ester (Hydrochloride)

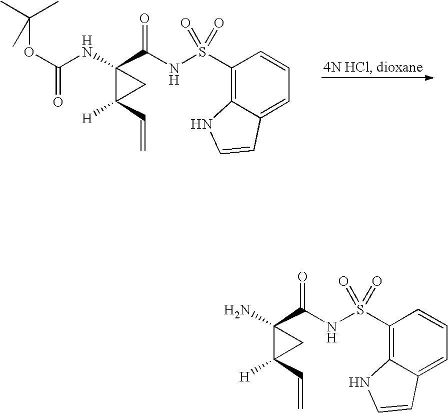

A mixture of 8.2 g (20 mmol) [(1R,2S)-1-(1H-Indole-7-sulfonylaminocarbonyl)-2-vinyl-cyclopropyl]-carbamic acid tert-butyl ester and 38 mL HCl (4 M in dioxane) in 38 mL dioxane is stirred at RT for 1.5 h. The mixture is concentrated under reduced pressure and coevaporated with DCM to give the title compound.

LC-MS (Method F): t_{R}=1.025, M+H=304.1

### EXAMPLE 8

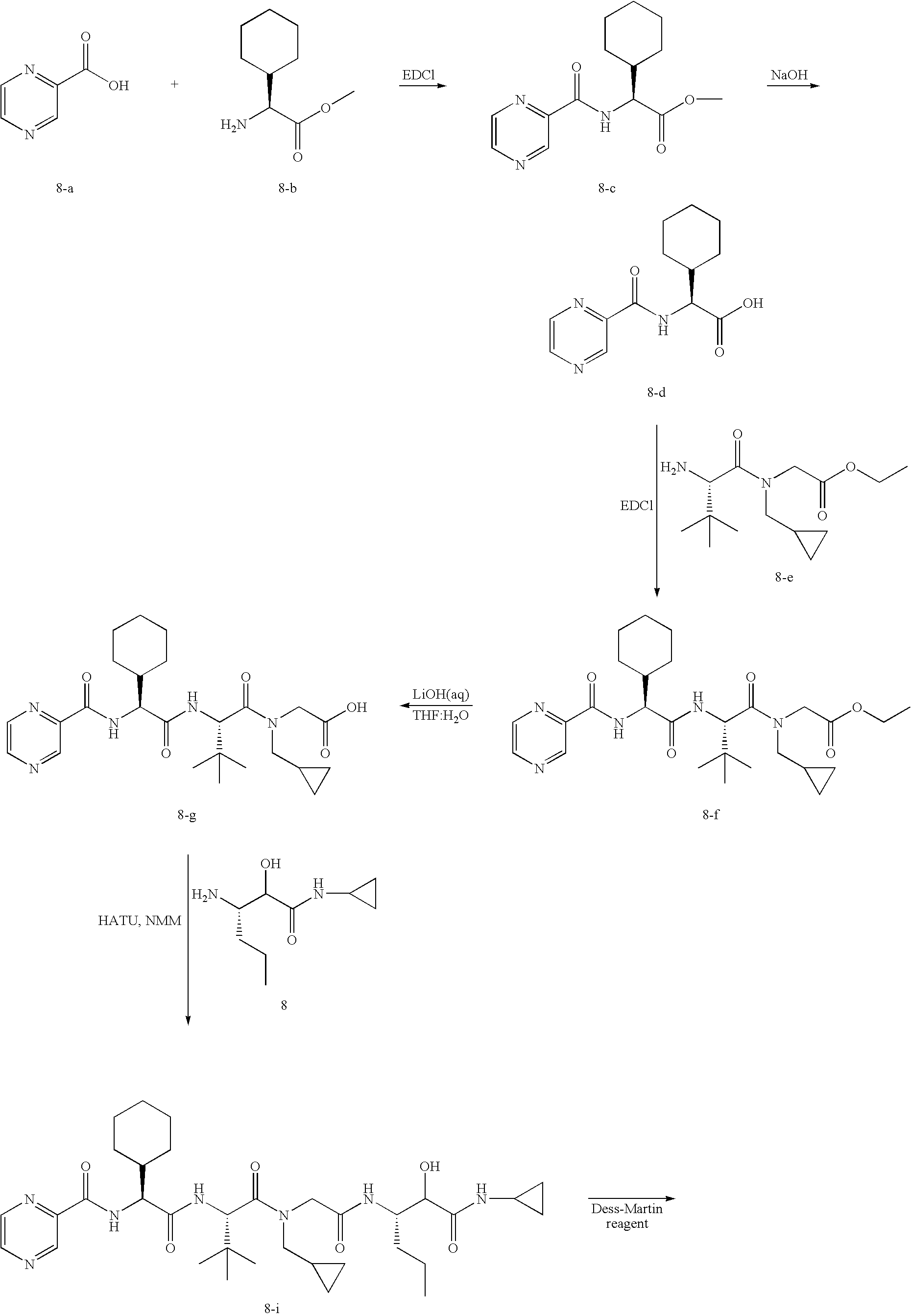

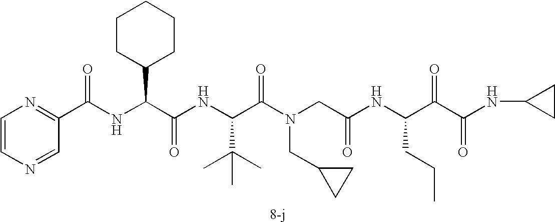

### Step 8-1: Synthesis of Compound 8-c

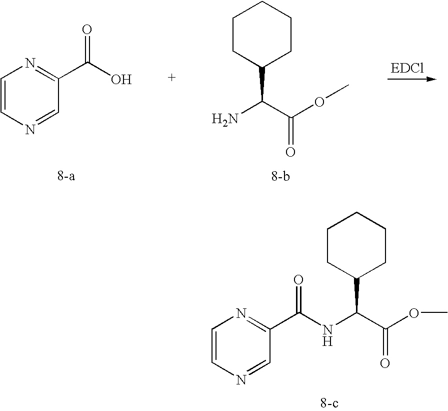

To a solution of 2-pyrazinecarboxylic acid (8-a1.44 g, 11.6 mmol) dissolved in THF (30 mL) at RT is added EDC (2.23 g, 11.6 mmol), HOBt (1.57 g, 11.6 mmol) and the solution stirred for 20 min. Cyclohexylglycine methyl ester (8-b, 2.0 g, 9.6 mmol, Cat# 12003, Chemilmpex), Diisopropylamine (2.5 g, 19.3 mmol) is added and the reaction stirred at overnight. The sample is then concentrated and then dissolved in EtOAc (100 mL) then washed with NH₄Cl (50 mL), NaHCO₃ (Sat'd, 50 mL), NaCl (Sat'd, 50 mL) then dried over MgSO₄ and concentrated to yield a yellow oil. The sample is then purified by FCC to furnish the white solid (2.6 g, 9.4 mmol).

ES-MS:[M+H]⁺=278.2

### Step 8-2: Synthesis of Compound 8-d

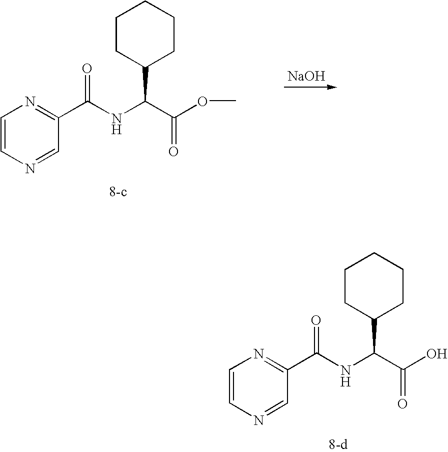

The ester (8-c 416 mg, 1.5 mmol) in MeOH (12 mL) is added NaOH (2N, 1.9 mL, 3.75 mmol) and the solution allowed to stir overnight at RT. The reaction mixture is then acidified with resin (IR-120 H⁺). The solids are filtered off and the filtrate concentrated to afford a solid (395 mg, 1.50 mmol). The acid is used directly in the next step.

ES-MS:[M+H]⁺=264.0.

### Step 8-3: Synthesis of Compound 8-f

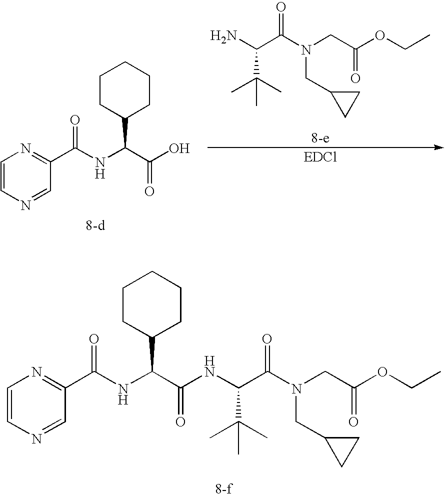

To a solution of acid (8-d, 249 mg, 0.90 mmol) dissolved in THF (2.8 mL) at RT is added EDC (172 mg, 0.90 mmol), HOBt (121 mg, 0.90 mmol) and the solution stirred for 20 min. Amine (8-e, 274 mg, 0.90 mmol) dissolved in DMF:THF (0.2 mL: 0.8 mL), Diisopropylamine (231 mg, 1.80 mmol) is added and the reaction stirred at RT overnight. The sample is then concentrated and then dissolved in EtOAc (50 mL) then washed with NH₄Cl (20 mL), NaHCO₃ (Sat'd, 20 mL), NaCl (Sat'd, 20 mL) then dried over MgSO₄ and concentrated to yield a yellow oil. The sample is then purified by FCC (EtOAc:Hexane 1:1) to furnish the product (406 g, 0.78 mmol).

ES-MS:[M+H]⁺=516.1

### Step 8-4: Synthesis of Compound 8-g

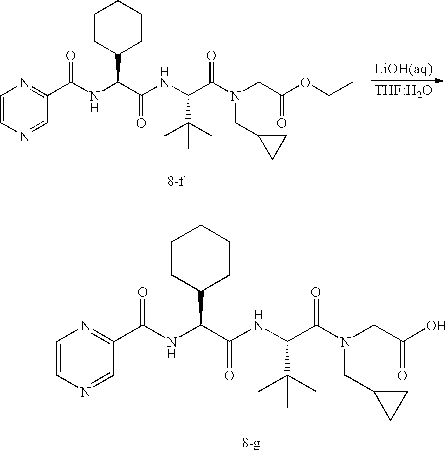

To a solution of the ethyl ester (8-f, 495 mg, 0.97 mmol) dissolved in THF: H2O (3:1, 3.9:1.3 mL) cooled at 0° C. is added LiOH (1.3 mL, 1.3 mmol, 1.3 M solution) dropwise over a 10 min interval. The solution is allowed to warm to RT and stirred overnight. The reaction mixture is then acidified with resin (IR-120 H⁺). The solids are filtered off and the filtrate concentrated to afford a solid (472 mg, 0.97 mmol). ES-MS:[M+H]⁺=488.1.

### Step 8-5: Synthesis of Compound 8-i

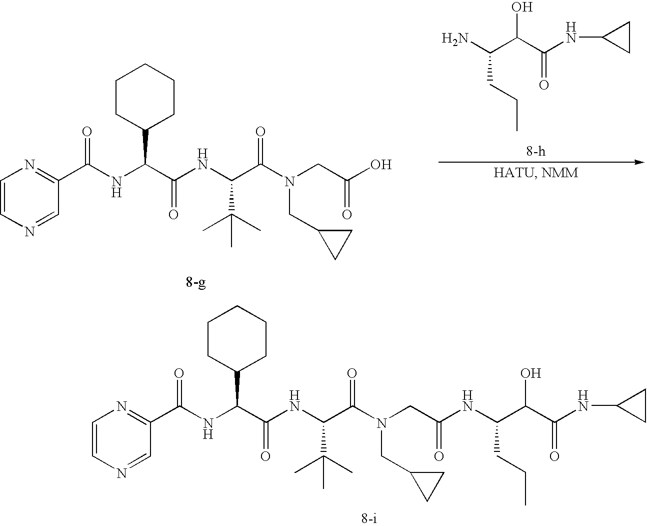

The acid (8-g, 474 mg, 0.98 mmol) is dissolved CH₂Cl₂:DMF (1:1 20 mL) and the solution cooled to 0° C. and treated with HATU (525 mg, 1.38 mmol). The amine (8-h, 218 mg, 1.17 mmol) is then added in small portions followed by dropwise addition of NMM (397 mg, 3.92 mmol). The reaction mixture is allowed to warm to RT and then stirred overnight. The sample is then concentrated and then dissolved in EtOAc (50 mL) then washed with Citric acid (20 mL, 10%), NaHCO₃ (Sat'd, 20 mL), NaCl (Sat'd, 20 mL) then dried over MgSO₄ and concentrated off whit solid. The sample is then purified by FCC (Acetone:Heptane 1:1) to furnish the product (614 mg, 0.91 mmol).

ES-MS:[M+H]⁺=656.4.

### Step 8-6: Synthesis of Compound 8-j

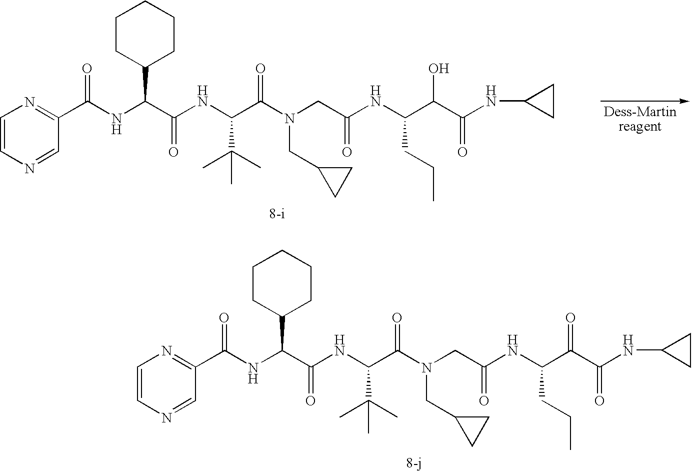

To a solution of the alcohol (8-i, 740 mg, 1.13 mmol) in DCM (24 mL) is added DMP reagent and the mixture is stirred at RT for 1 h. The mixture is then filtered through a pad of celite. The celite is washed with additional DCM (2×20 mL) and the combined filtrate washed with Na₂SO₃ (15 mL, 1M solution), NaHCO₃ (sat'd, 15 mL), NaCl (sat'd, 15 mL) then dried over MgSO₄ and evaporated to dryness. FCC (Acetone:Heptane 1:1) gave a white solid (309 mg, 0.47 mmol).

ES-MS:[M+H]⁺=654.3.

### EXAMPLE 9

### Example 9 is Not a Final Compound, But an Intermediate (Remove!)

### Preparation of Intermediate 8-e in Example 8

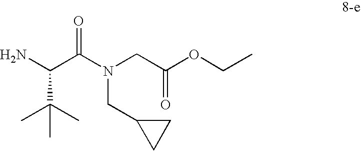

### Step 9-1: Synthesis of Compound 9-b

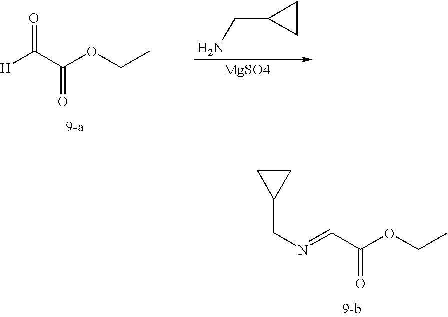

To a 250 mL round bottom flask containing CH₂Cl₂ (40 mL) is added Cyclopropanemethylamine (10 g, 138 mmol), MgSO₄ (6 g, 44 mmol) and the mixture stirred for 5 mins under Nitrogen. Ethyl glyoxalate (9-a, 27.6 g, 138 mmol) is added slowly over the course of 10 min. The reaction is then stirred at RT for 2 h. The slurry is then filtered and the filtrate concentrated to afford orange oil (22 g, 138 mmol).

### Step 9-2: Synthesis of Compound 9-c

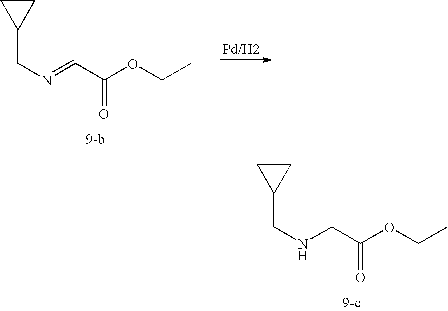

The Imine (9-b, 20.3 g, 128 mmol) is dissolved in EtOAc (45 mL) and purged with N₂. Palladium (10% on Activated C, 8.18g, 76.9 mmol) is added and the reaction purged under N₂ again. The solution is then purged under H₂ and the reaction stirred overnight at RT. The reaction mixture is then filtered through a pad of celite and the filtrate concentrated and then purified by FCC to yield a yellow oil (15 g, 95 mmol), The product is purified by FCC to afford a yellow oil.

### Step 9-3: Synthesis of Compound 9-d

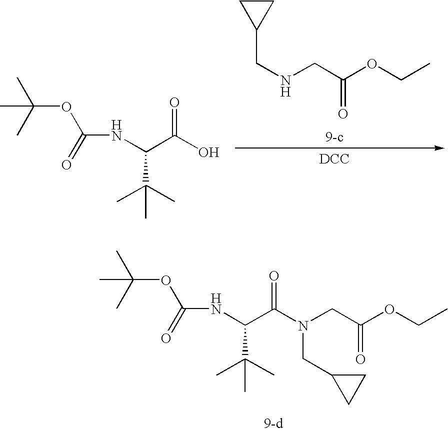

The acid:Boc-L-tert-leucine (270 mg, 1.72 mmol) is dissolved in a mixture of CH₂Cl₂ (7 mL). DCC (390 mg, 1.89 mmol) followed by HOBt (255 mg, 1.89 mmol) is added and the reaction stirred for 30 min. To this solution is then added the cyclopropylmethylamine glycine methlyester (9-c, 438 mg, 1.89 mmol, dissolved in 3.5 mL THF) and the reaction allowed to stir overnight at RT. The mixture is then filtered through a sintered glass funnel (fine) and the filter cake washed with CH₂Cl₂ (2×10 mL). The filtrate is concentrated to furnish the product which is purified by FCC (EtOAc:Hexane, 1:5), (210 mg, 0.58 mmol).

ES-MS:[M+H]⁺=371.1.

### Step 9-4: Synthesis of Compound 8-e

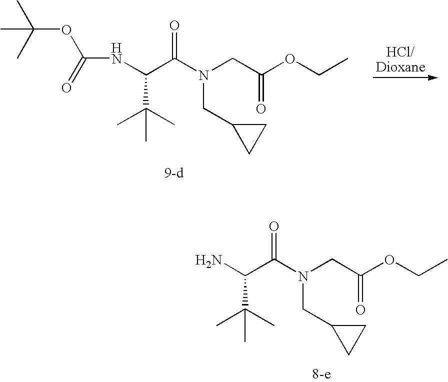

To a solution of the ethyl ester (9-d, 188 mg, 0.51 mmol) is dissolved in dioxane (1.2 mL) and the solution cooled to ° C. A solution of 4N HCl in Dioxane (1.3 mL, 5.09 mmol) is added and the mixture stirred at RT overnight. The mixture is then evaporated to yield a white solid (155 mg, 0.51 mmol) which is used without purification.

ES-MS: [M+H]⁺=271.1.

### EXAMPLE 10

### (S)-2-(3-tert-Butyl-ureido)-N-[(2-carbamoyl-1-cyclobutylmethyl-2-oxo-ethylcarbamoyl)-methyl]-3,3-dimethyl-N-(1-phenyl-cyclopropylmethyl)-butyramide

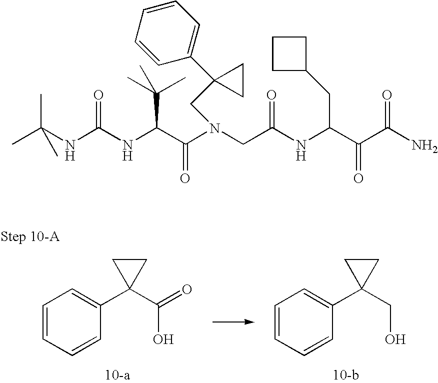

Carbonyl diimidazole (2 g, 12.3 mmol) is added to a solution of 1-phenylcyclopropanecarboxylic acid (10-a, 2.0 g, 12.3 mmol) in THF (20 mL) and the mixture is stirred at room temperature for 10 minutes. A solution of sodium borohydride (744 mg, 19.7 mmol) in water (8 mL) is added and the mixture is stirred at room temperature overnight. It is quenched by addition of HCl (1M) and the product is extracted into ethyl acetate (2×100 mL). The combined organic extracts are washed with aqueous saturated sodium bicarbonate solution and brine, dried over MgSO₄ and concentrated in vacuo to give the product as a colourless liquid (1.49 g).

¹H NMR (CDCl₃): δ 7.4-7.2 (m, 5H), 3.7 (s, 2H), 0.9 (m, 2H), 0.85 (m, 2H).

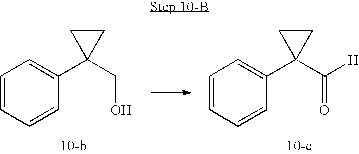

A solution of 10-b (1.49 g, 10 mmol) in dichloromethane (10 mL) is added in one portion to a suspension of pyridinium chlorochromate (3.26 g, 15 mmol) and celite in dry dichloromethane (15 mL). The resultant mixture is stirred at room temperature under nitrogen for 2 hours. The solid is removed by filtration and washed with further dichloromethane. The filtrate is evaporated to dryness and the residue is purified by chromatography on silica eluting with a mixture of ethyl acetate and cyclohexane (1:1) to give the product 10-c as a pale yellow liquid (1.1 g).

¹H NMR (CDCl₃): δ 9.3 (s, 1H), 7.4-7.25 (m, 5H), 1.6 (m, 2H), 1.4 (m, 2H).

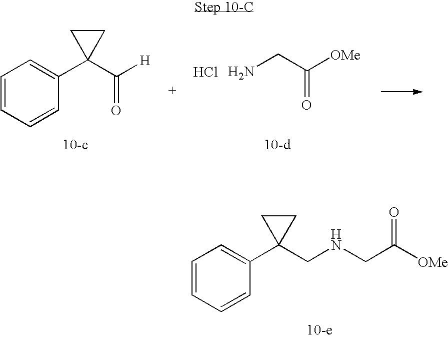

Triethylamine (1.36 mL, 990 mg, 9.8 mmol) and glycine methyl ester hydrochloride (10-d, 1.04 g, 8.3 mmol) are added to a solution of 10-c (1.1 g, 7.5 mmol) in methanol (10 mL) and molecular sieve (4A). The resultant mixture is stirred at room temperature overnight then cooled to 0° C. Sodium borohydride (371 mg, 9.8 mmol) is added in portions and the mixture is stirred at room temperature for 2 hours. Water is added and the mixture is extracted with dichloromethane, washed with brine, dried over MgSO₄ and filtered. The filtrate is evaporated to dryness to give the product 10-e as a colourless liquid (1.33 g).

Found m/z ES+=220.

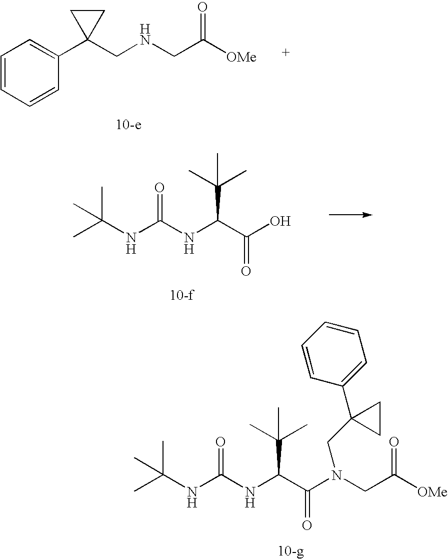

A stirred solution of 10-f (935 mg, 3.4 mmol) in a mixture of dichloromethane (7 mL) and N,N-dimethylformamide (7 mL) under nitrogen is cooled to 0° C. and treated with HATU (1.87 g, 4.9 mmol). 10-e (900 mg, 4.1 mmol) is then added in portions followed by dropwise addition of N-methylmorpholine (1.81 mL, 1.65 g, 16.4 mmol). The mixture is allowed to warm to room temperature and stirred for 6 hours. It is concentrated in vacuo and the residue is dissolved in ethyl acetate and washed with aqueous citric acid solution (10%), saturated aqueous sodium bicarbonate solution and brine then dried over MgSO₄. It is filtered and the filtrate is evaporated to dryness. The residue is purified by chromatography on silica (gradient:ethyl acetate and cyclohexane 1:95 to 1:4) to give the product 10-g as a white foam (803 mg).

Found m/z ES+=432.

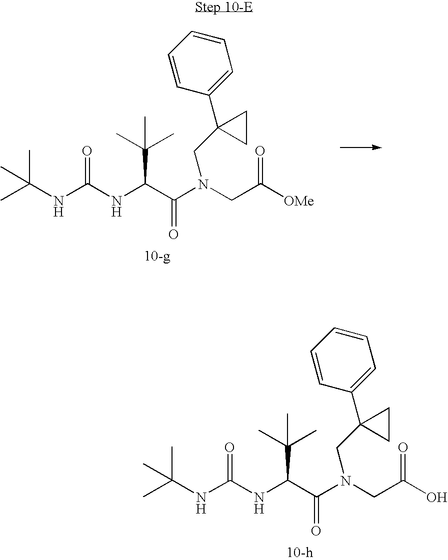

10-g (804 mg, 1.9 mmol) is dissolved in a mixture of THF (4 mL) and water (1.3 mL) and the solution is cooled to 0° C. An aqueous solution of lithium hydroxide (1.3 M, 1.8 mL, 2.4 mmol) is added slowly. The resultant mixture is allowed to warm to room temperature and stirred for 2 hours. The mixture is treated with hydrochloric acid (1M) and extracted with ethyl acetate, washed with brine, dried over MgSO₄ and filtered. The filtrate is evaporated to dryness to give the product 10-h as a white solid (726 mg).

Found m/z ES+=418.

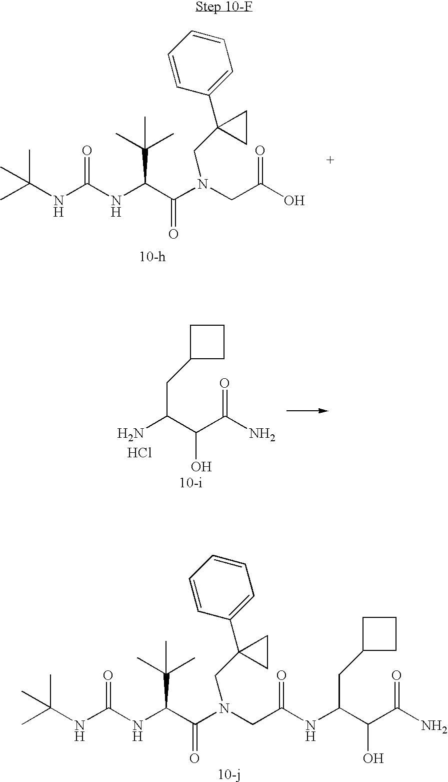

10-i (195 mg, 0.931 mmol) is suspended in a mixture of dichloromethane (4 mL) and N,N-dimethylformamide (4 mL) and the mixture is cooled to 0° C. HATU (532 mg, 1.4 mmol) is added followed by 10-h (324 mg, 0.776 mmol) and finally by N-methylmorpholine (0.32 mL, 293 mg, 2.91 mmol). The resultant mixture is stirred at room temperature for 7 hours. It is concentrated in vacuo and the residue is dissolved in ethyl acetate, washed with aqueous citric acid solution (10%), saturated aqueous sodium bicarbonate solution and brine, dried over MgSO₄ and filtered. The filtrate is evaporated to dryness and the residue is purified by chromatography on silica (gradient:acetone and pentane 1:4 to 1:1) to give the product 10-j as a white solid (380 mg).

Found m/z ES+=572 and ES-570.

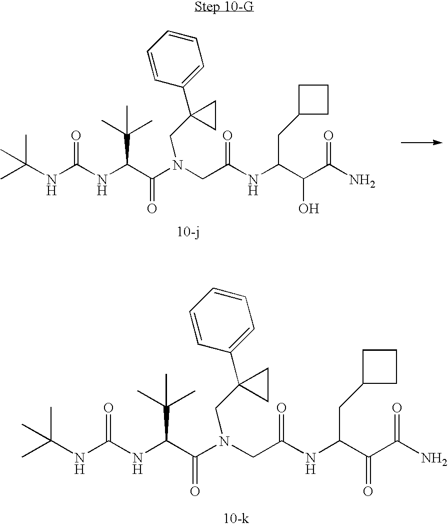

A solution of sulphur trioxide-pyridine complex (64 mg, 0.4 mmol) in dry DMSO (1 mL) is added to a solution of 10-j (114 mg, 0.2 mmol) and N,N-di-isopropyl N-ethyl amine (0.141 mL, 105 mg, 0.8 mmol) in dry DMSO (1 mL) and the mixture is stirred at room temperature overnight. Further sulphur trioxide-pyridine complex (100 mg, 0.63 mmol) is added and the mixture is stirred for 5 hours. Further N,N-di-isopropyl N-ethyl amine (0.15 mL, 111 mg, 0.86 mmol) and sulphur trioxide-pyridine complex (60 mg, 0.38 mmol) are added and the mixture is stirred at room temperature overnight. Aqueous ammonium chloride solution is added and the mixture is extracted with ethyl acetate, washed with brine, dried over MgSO₄ and filtered. The filtrate is evaporated to dryness and the residue is purified by chromatography on silica (gradient: acetone and pentane 1:4 to 1:1) to give the product 10-k as a white solid (40 mg).

Found m/z ES+=570.

### EXAMPLE 11

### Synthesis of (S)-2-(3-tert-Butyl-ureido)-N-[(2-carbamoyl-1-cyclobutylmethyl-2-oxo-ethylcarbamoyl)-methyl]-3,3-dimethyl-N-(1-phenyl cyclopentyl methyl)-butyramide

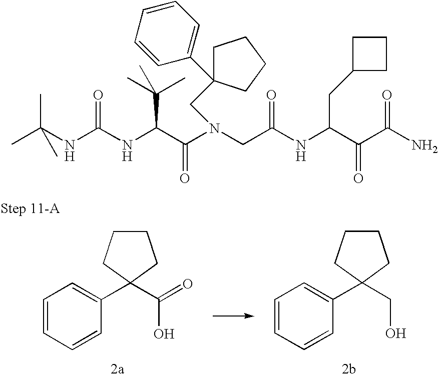

11-b is prepared from 11-a by proceeding in a manner similar to that used for the preparation of 10-b (Step 10-A).

¹H NMR (CDCl₃): δ 7.35-7.1 (m, 5H), 3.45 (s, 2H), 1.95 (m, 2H), 1.8 (m, 2H), 1.65 (m, 4H).

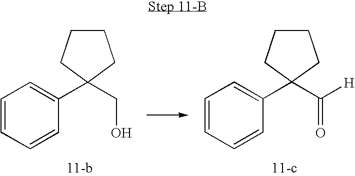

11-c is prepared from 11-b by proceeding in a manner similar to that used for the preparation of 10-c (Step 10-B).

¹H NMR (CDCl₃): δ 9.4 (s, 1H), 7.4-7.2 (m, 5H), 2.55 (m, 2H), 1.9 (m, 2H), 1.75 (m, 2H), 1.65 (m, 2H).

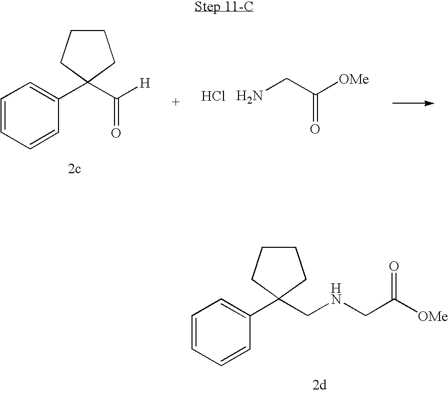

11-d is prepared from 11-c and 10-d by proceeding in a manner similar to that used for the preparation of 10-e (Step 10-C).

¹H NMR (CDCl₃): δ 7.3 (m, 4H), 7.2 (m, 1H), 3.65 (s, 3H), 3.25 (s, 2H), 2.7 (s, 2H), 2.0 (m, 2H), 1.9 (m, 2H), 1.7 (m, 4H).

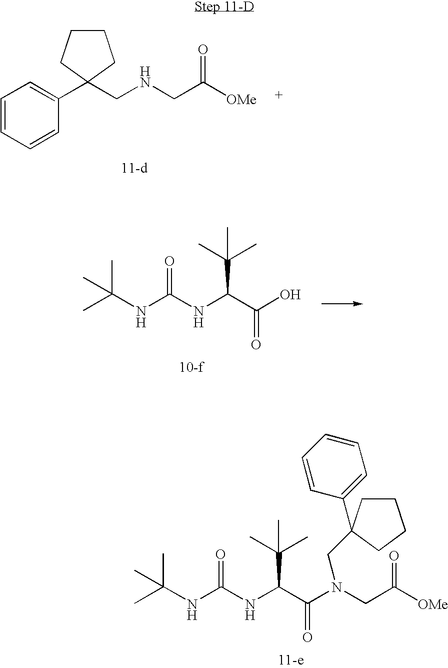

11-e is prepared from 11-d and 10-f by proceeding in a manner similar to that used for the preparation of 10-g (Step 10-D).

Found m/z ES+=460.

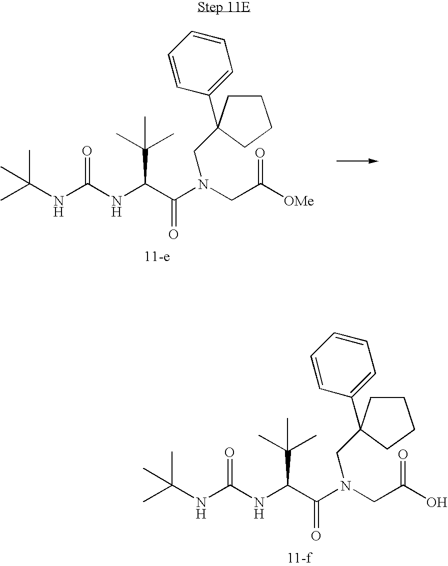

11-f is prepared from 11-e by proceeding in a manner similar to that used for the preparation of 10-h (Step 10-E).

Found m/z ES+=446.

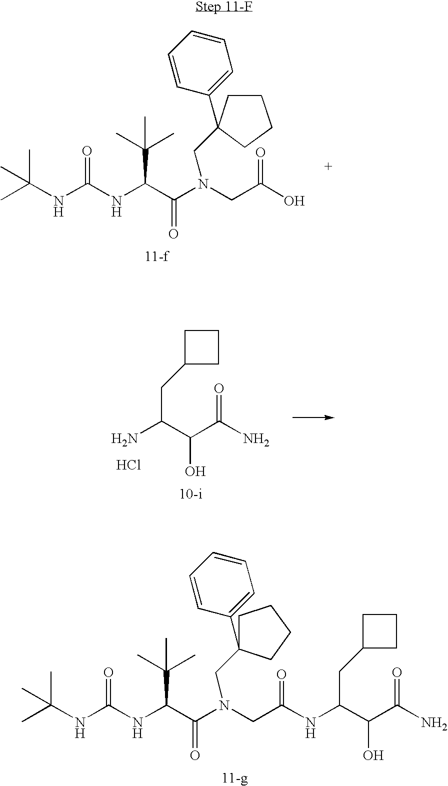

11-g is prepared from 11-f and 10-i by proceeding in a manner similar to that used for the preparation of 10-j (Step 10-F).

Found m/z ES+=400.

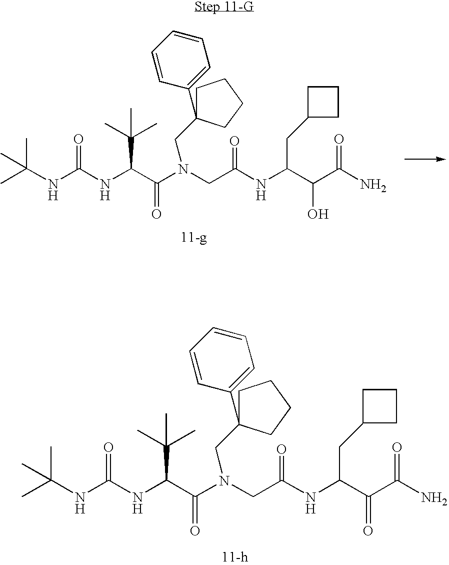

A solution of sulphur trioxide-pyridine complex (281 mg, 1.76 mmol) in dry DMSO (1.5 mL) is added to a solution of 11-g (151 mg, 0.25 mmol) and N,N-di-isopropyl N-ethyl amine (0.37 mL, 275 mg, 2.12 mmol) in dry DMSO (1.5 mL) under an atmosphere of nitrogen. The mixture is stirred at room temperature under nitrogen for 2 hours. Ammonium chloride is added and the mixture is extracted with ethyl acetate, washed with brine, dried over MgSO₄ and filtered. The filtrate is evaporated to dryness and the residue is purified by chromatography on silica (gradient:acetone and heptane 1:95 to 3:7). The resultant product is purified by dissolving in dichloromethane and precipitating the product by addition of petroleum ether to give the product 11-h as a white solid (20 mg).

Found m/z ES+=598.

### Biological Activity

### EXAMPLE 12

### HCV NS3-4A Protease Assay

The inhibitory activity of certain compounds of Table A against HCV NS3-4A serine protease is determined in a homogenous assay using the full-length NS3-4A protein (genotype 1a, strain HCV-1) and a commercially available internally-quenched fluorogenic peptide substrate as described by Taliani, M., et al. 1996 Anal. Biochem. 240:60-67, which is incorporated by reference in its entirety.

### EXAMPLE 13

### Luciferase-Based HCV Replicon Assay

The antiviral activity and cytotoxicity of certain compounds of Table A is determined using a subgenomic genotype 1b HCV replicon cell line (Huh-Luc/neo-ET) containing a luciferase reporter gene, the expression of which is under the control of HCV RNA replication and translation. Briefly, 5,000 replicon cells are seeded in each well of 96-well tissue culture plates and are allowed to attach in complete culture media without G418 overnight. On the next day, the culture media are replaced with media containing a serially diluted compound of Table A in the presence of 10% FBS and 0.5% DMSO. After a 48-h treatment with the compound of Table A, the remaining luciferase activities in the cells are determined using BriteLite reagent (Perkin Elmer, Wellesley, Mass.) with a LMaxII plate reader (Molecular Probe, Invitrogen). Each data point represents the average of four replicates in cell culture. IC₅₀ is the concentration of the at which the luciferase activity in the replicon cells is reduced by 50%. The cytotoxicity of the compound of Table A is evaluated using an MTS-based cell viability assay.

Compounds Table A supra have been tested in at least one of the protease assay of Example 12 or the replicon assay of Example 13 and exhibit an IC₅₀ of less than about 10 μM or less in at least one of the assays recited in Example 12 and 13.

### Equivalents

Those skilled in the art will recognize, or be able to ascertain using no more than routine experimentation, many equivalents to the specific embodiments and methods described herein. Such equivalents are intended to be encompassed by the scope of the following claims.

What is claimed is:

1. A compound of the Formula I: 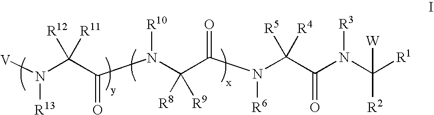
and pharmaceutically acceptable salts and stereoisomers thereof, wherein
x is 0 or 1;
y is 0 or 1;
R¹, R², R⁴, R⁵, R⁶, W, R¹³ and V are each, independently, selected from hydrogen or from the group consisting of alkyl, aralkylaralkyl, heteroalkyl, heterocyclyl, heteroaryl, aryl-heteroaryl, alkyl-heteroaryl, cycloalkyl, alkyloxy, aralkylaralkyloxy, aryloxy, heteroaryloxy, heterocyclyloxy, cycloalkyloxy, amino, mono- and di-alkylamino, arylamino, aralkylamino, heteroarylamino, cycloalkylamino, carboxyalkylamino, arlylalkyloxy and heterocyclylamino; each of which may be further independently substituted one or more times with X¹ and X²; wherein X¹ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl; wherein X¹ can be independently substituted with one or more of X² moieties which can be the same or different and are independently selected; wherein X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;
W is also selected from the group consisting of C(O)OH, C(O)OR²⁴, C(O)-amine, C(O)—C(O)OH, C(═N—O—R²⁴)—C(O)-amine, C(O)N(H)S(O)₂R²⁴, C(O)—C(O)-amine, CON(H)SO₂-amine and C(O)—[C(O)]ₐ-heterocycle, wherein the heterocycle may be substituted or unsubstituted, wherein a is 0 or 1, wherein each R²⁴ is independently selected from the group consisting of H, halogen, hydroxy, COOH, amino, C(O)NH₂, C₁₋₄-alkyl, C₃₋₆-cycloalkylC₀₋₄alkyl, C₃₋₆-cycloalkylC₀₋₄alkoxy, mono- and diC₁₋₄alkylamino, aryl, aryloxy, aralkyl, aralkyloxy, heterocycleC₀₋₄alkyl, and heterocycleC₀₋₄alkoxy;
V is also selected from the group consisting of -Q¹-Q², wherein Q¹ is absent, C(O), N(H), N(C₁₋₄-alkyl), C═N(CN), C═N(SO₂CH₃), or C═N—COH, and Q² is H or is selected from the group consisting of C₁₋₄-alkyl, O—C₁₋₄-alkyl, NH₂, N(H)—C₁₋₄-alkyl, N(C₁₋₄-alkyl)₂, SO₂-aryl, SO₂-C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄-alkyl substituted by one or more halogen atoms, or C₃₋₆-cycloalkyl;
R³, R⁸, R⁹, R¹⁰, R¹¹ and R¹³ are each, independently, selected from the group consisting of H, C₁₋₄-alkyl and C₃₋₆cycloalkylC₀₋₄alkyl; and
R¹² is selected from the group consisting of H, C₁₋₄-alkyl, C₃₋₆cycloalkylC₀₋₄alkyl and aryl;
or R¹ and R² may together form a 3, 4, 5, 6 or 7-membered ring that is aromatic or non-aromatic and may contain one or more heteroatoms, wherein the ring may be further substituted one or more times;
or R¹¹ and V may together form a 3, 4, 5, 6 or 7-membered ring that is aromatic or non-aromatic and may contain one or more additional heteroatoms, wherein the ring may be further substituted one or more times;
or when x and y are 0, R⁶ and V may together form a 3, 4, 5, 6 or 7-membered ring that is aromatic or non-aromatic and may contain one or more additional heteroatoms, wherein the ring may be further substituted one or more times.

2. The compound of claim 1, wherein
y is 0 or 1;
R¹ is selected from the group consisting of H and C₁₋₄-alkyl;
R² is selected from the group consisting of C₁₋₄-alkyl, C(O)C₁₋₄-alkyl, C(O)OC₁₋₄-alkyl, and C₃₋₆cycloalkylC₀₋₄alkyl;
or R¹ and R² may together form a 3, 4, 5, 6 or 7-membered ring that is aromatic or non-aromatic and may contain one or more heteroatoms, wherein the ring may be further substituted one or more times;
W is also selected from the group consisting of C(O)OH, C(O)OR²⁴, C(O)-amine, C(O)—C(O)OH, C═N—O—R²⁴)—C(O)-amine, C(O)N(H)S(O)₂ R²⁴, C(O)—C(O)-amine, CON(H)SO₂-amine and C(O)—[C(O)]ₐ-heterocycle, wherein the heterocycle may be substituted or unsubstituted, wherein a is 0 or 1, wherein each ^{R24} is independently selected from the group consisting of H, halogen, hydroxy, COOH, amino, C(O)NH₂, C₁₋₄-alkyl, C₃₋₆-cycloalkylC₀₋₄alkyl, C₃₋₆-cycloalkylC₀₋₄alkoxy, mono- and diC₁₋₄alkylamino, aryl, aryloxy, aralkyl, aralkyloxy, heterocycleC₀₋₄alkyl, and heterocycleC₀₋₄alkoxy;
R³ is selected from the group consisting of H and C₁₋₄-alkyl;
R⁴ and R⁶ are each, independently, selected from the group consisting of H, C₁₋₄-alkyl, C₃₋₆-cycloalkyl, C₃₋₆cycloalkylC₀₋₄alkyl, aryl, aralkyl and heterocycle, each of which may be independently substituted one or more times;
R⁵ is H;
R⁸, R¹⁰ and R¹¹ are each, independently, selected from the group consisting of H and C₁₋₄-alkyl;
R¹³ are H;
R⁹ is selected from the group consisting of H, C₁₋₄-alkyl and C₃₋₆-cycloalkyl;
R¹² is selected from the group consisting of H, C₁₋₄-alkyl, C₃₋₆-cycloalkyl and aryl; and
V is selected from the group consisting of -Q¹-Q², wherein Q¹ is absent, C(O), S(O)₂, N(H), N(C₁₋₄-alkyl), C═N(CN), C═N(SO₂CH₃), C═N—COH, or C═N—COC₁₋₄alkyl, and Q² is H or is selected from the group consisting of C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄-alkyl substituted by one or more halogen atoms, or C₃₋₆-cycloalkyl;
or R¹¹ and V form the following 5-membered ring which may be further substituted:

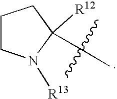

3. The compound of claim 1, wherein R¹¹ and V form the following structure:

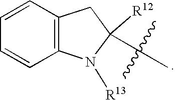

4. The compound of claim 2, wherein R¹⁰ is C(O)C₁₋₄-alkyl.

5. The compound of claim 2, wherein R¹² is

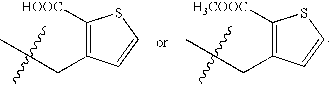

6. The compound of claim 1, wherein R⁶ is selected from the group consisting of H, CH₂-cyclopentyl, CH₂-cyclopropyl, cyclopentyl, cyclopropyl and benzyl.

7. The compound of claim 1, wherein R¹² is selected from the group consisting of t-butyl and cyclohexyl.

8. The compound of claim 1, wherein R⁸ is selected from the group consisting of H and t-butyl.

9. The compound of claim 1, wherein Formula I is represented by a compound of the Formula II: 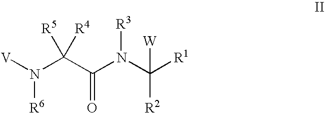
wherein R¹, R², R³, R⁴, R⁵, R⁶, W and V have the meanings set forth for Formula I.

10. The compound of claim 9, wherein R⁴ and R⁵ are H.

11. The compound of claim 9, wherein V is —C(O)CH₃ or

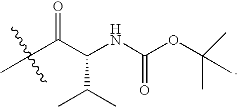

12. The compound of claim 9, wherein R⁶ is CH₂-cyclopentyl or CH₂-naphthyl.

13. The compound of claim 9, wherein R⁶ and V form together the following 6-membered ring:

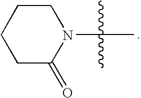

14. The compound of any one of the above claims, wherein R² is selected from the group consisting of pentyl and CH₂-cyclobutyl.

15. The compound of any one of the above claims, wherein R² is selected from the group consisting of propyl and 2-cyclobutyl-ethyl.

16. The compound of claim 1, wherein R¹¹ is H and R¹² is C₃₋₆-cycloalkyl.

17. The compound of any one of the above claims, wherein W, R¹ and R² form a substituent of the following formulas: 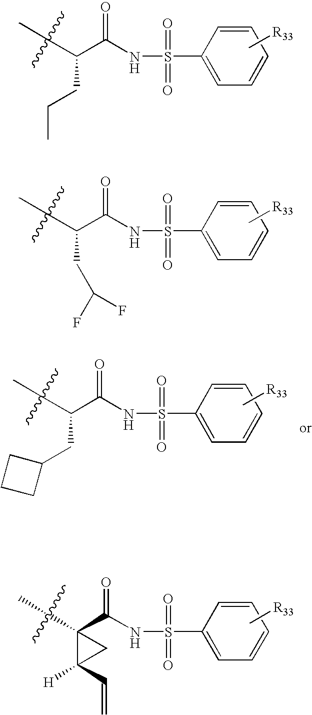
wherein R³³ is selected from the group consisting of H, phenyl, methyl, CF₃, tBu, NO₂, Cl, CN, NH₂, OH, NHCH₃, NHCH₂CH₃, NHCH(CH₃)₂, OCH₃, NHPh, OPh, NHCOCH₃, NHCOPh, OCH2Ph, COCH₃, CO₂Et, CO₂CH₃, CONHPh and CONHCH₃, or R³³ can be a ring fused which taken in combination with the phenyl ring form a naphthyl ring system or a indolyl ring system.

18. The compound of any one of the above claims, wherein W, R¹ and R² form substituents selected from the group consisting of

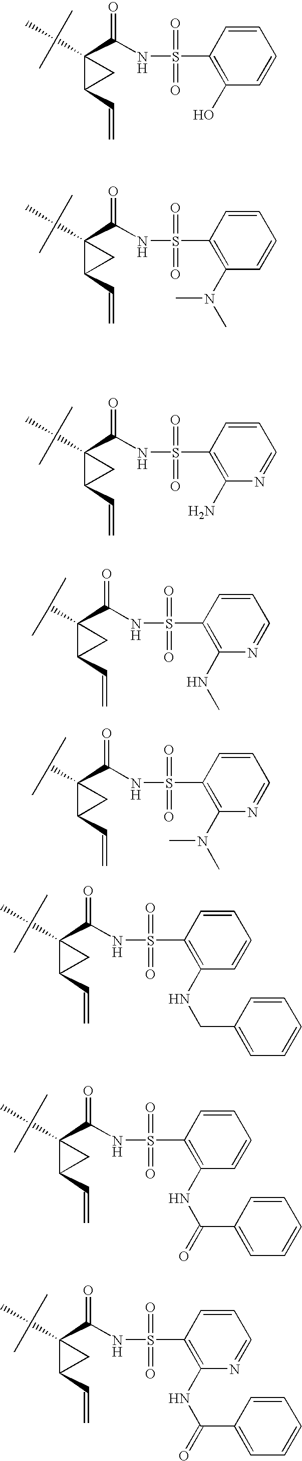

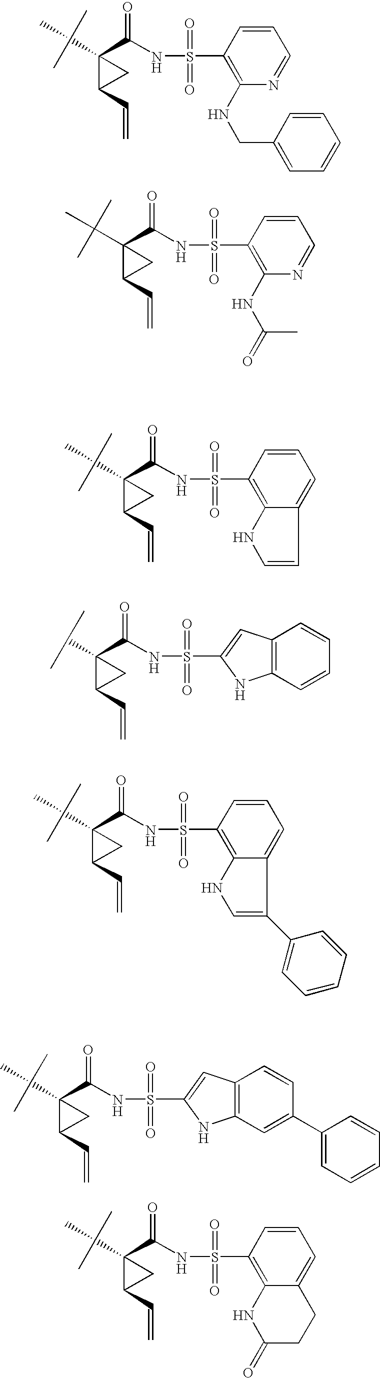

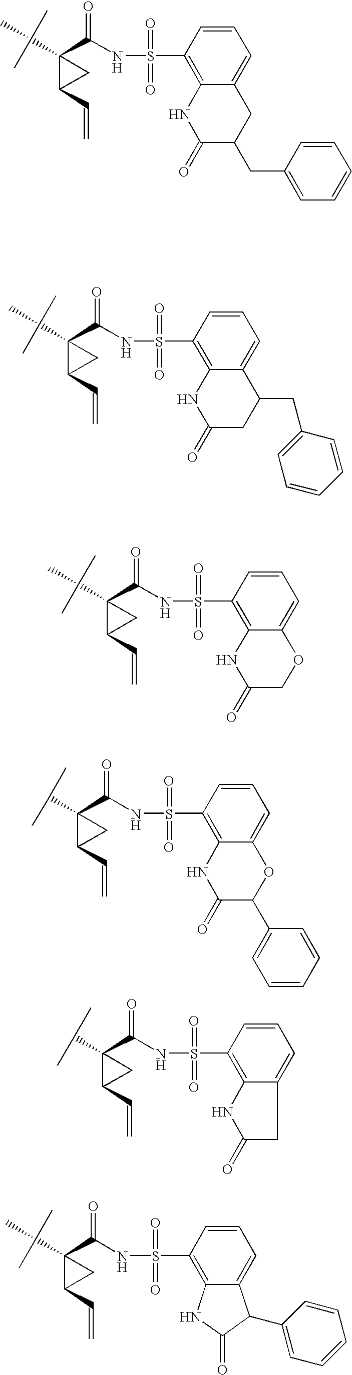

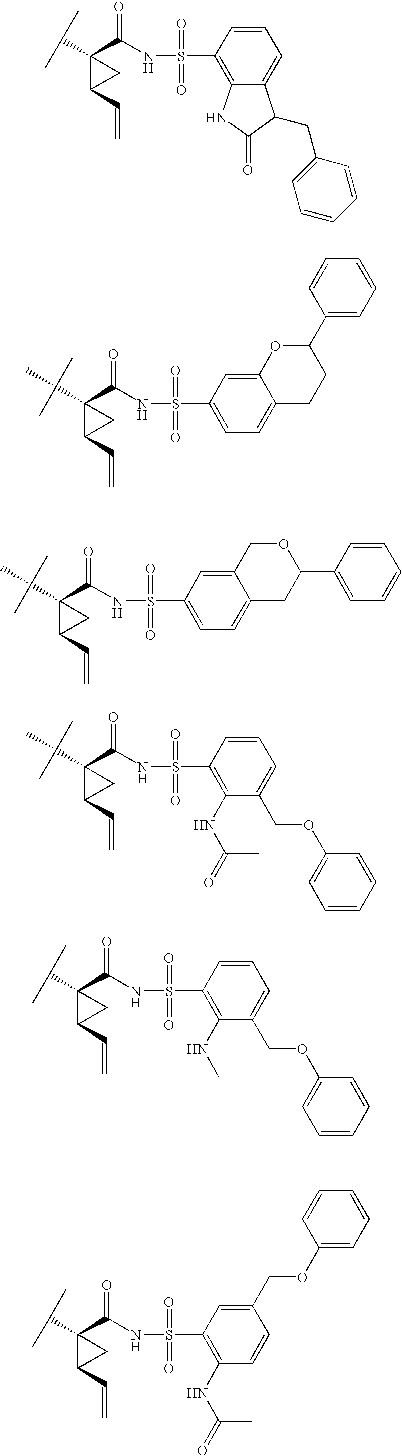

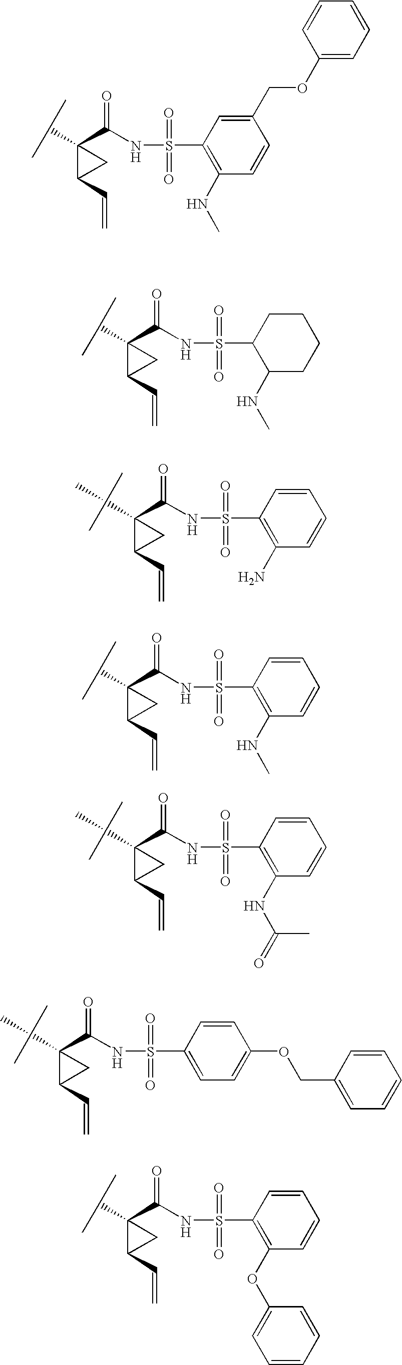

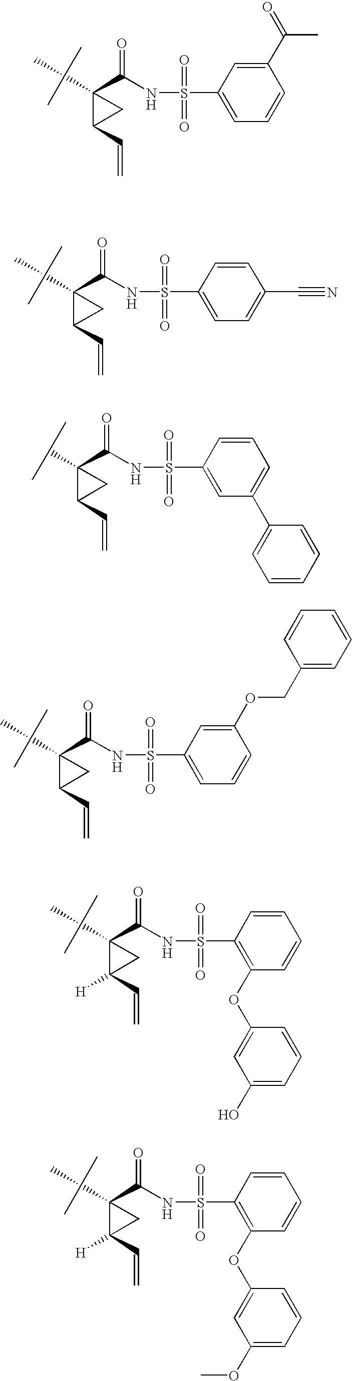

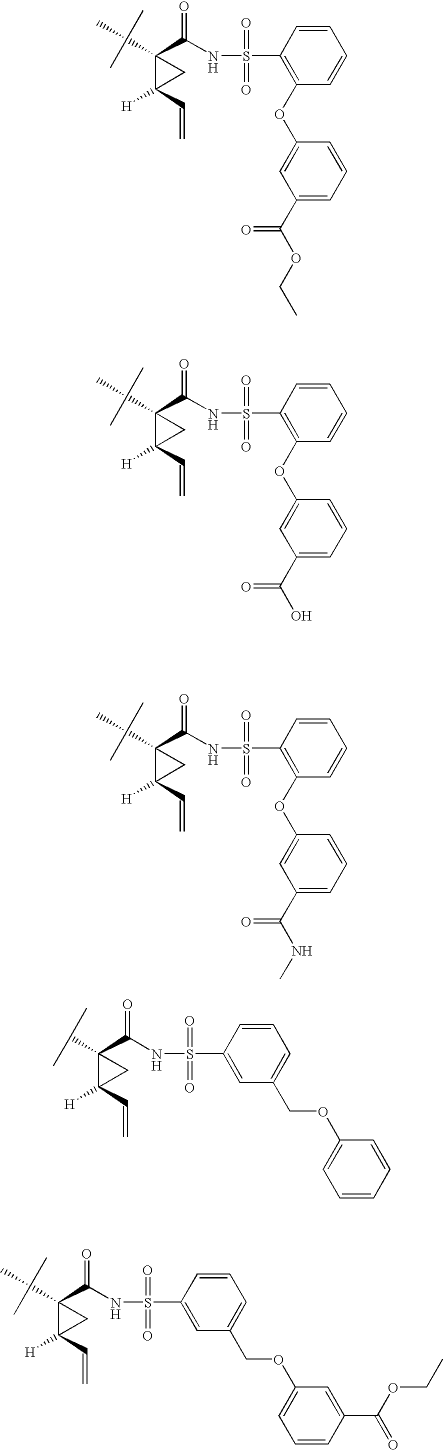

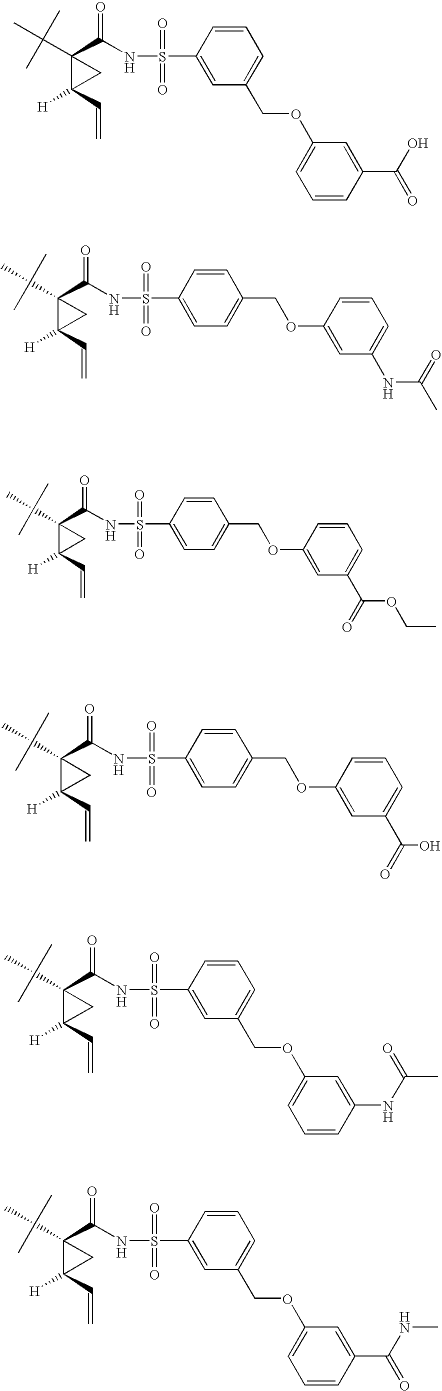

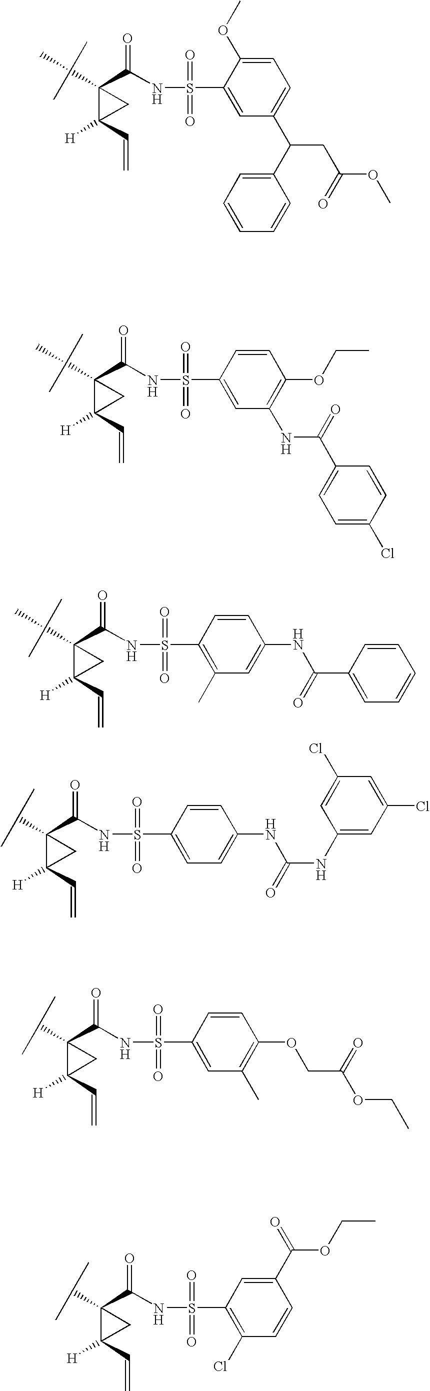

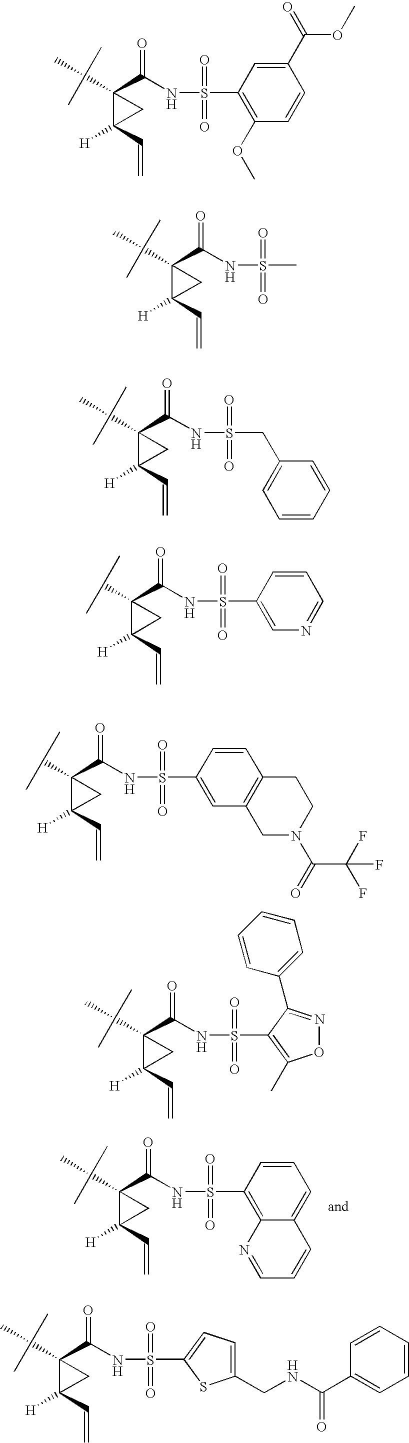

19. The compound of any one of the above claims, wherein any of the heterocycle groups are independently selected from the group consisting of acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline, benzoimidazolyl, benzofuranyl, benzofurazanyl, benzopyrazolyl, benzotriazolyl, benzothiophenyl, benzoxazolyl, carbazolyl, carbolinyl, cinnolinyl, fuiranyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, each of which may be independently further substituted one or more times with a halogen atom, C₁₋₄-alkyl, C₁₋₄-alkyl substituted by one or more halogen atoms, or C₃₋₆-cycloalkyl.

20. The compound of any one of the above claims, wherein W is C(O)—C(O)—N(H)-cyclopropyl or C(O)—C(O)—N(H)—NH₂.

21. The compound of any one of the above claims, wherein V is selected from the group consisting of C(O)R²⁴, C(O)N(H)R²⁴ and C(O)OR²⁴, wherein each R²⁴ is independently selected from the group consisting of H, halogen, hydroxy, COOH, amino, C(O)NH₂, C₁₋₄-alkyl, C₃₋₆-cycloalkylC₀₋₄alkyl, C₃₋₆-cycloalkylC₀₋₄alkoxy, mono- and diC₁₋₄alkylamino, aryl aryloxy, aralkyl, aralkyloxy, heterocycleC₀₋₄alkyl, and heterocycleC₀₋₄alkoxy.

22. The compound of any one of the above claims, wherein V is selected from the group consisting of benzyl, substituted benzyl, naphthyl, C₁₋₄-alkyl, and

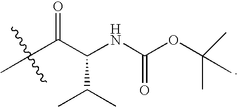

23. The compound of any one of the above claims, wherein any of the C₃₋₆-cycloalkyl groups may be independently substituted one or more times with a halogen atom, aryl, trihalomethyl, or C₁₋₄-alkyl.

24. The compound of any one of the above claims, wherein W is selected from the group consisting of C(O)—C(O)N(R²³)₂, wherein R²³ is independently selected from hydrogen or from the group consisting of C₁₋₄-alkyl, C₃₋₆-cycloalkylC₀₋₄alkyl, aryl and heterocycle, each of which may be independently substituted one or more times with a halogen atom or C₁₋₄-alkyl.

25. The compound of any one of the above claims, wherein W is selected from the group consisting of C(O)—C(O)NH₂, C(O)—C(O)N(H)-cyclopropyl, C(O)-benzothiazole, C(O)-benzoimidazole, C(O)-oxazole, C(O)-imidazole, and C(O)-oxadiazole, wherein the benzothiazole, benzoimidazole, oxazole and oxadiazole groups may be independently substituted one or more times with a halogen atom, aryl, trihalomethyl, C₃₋₆-cycloalkylC₀₋₄alkyl or C₁₋₄-alkyl.

26. The compound of any one of the above claims, wherein W is selected from the group consisting of 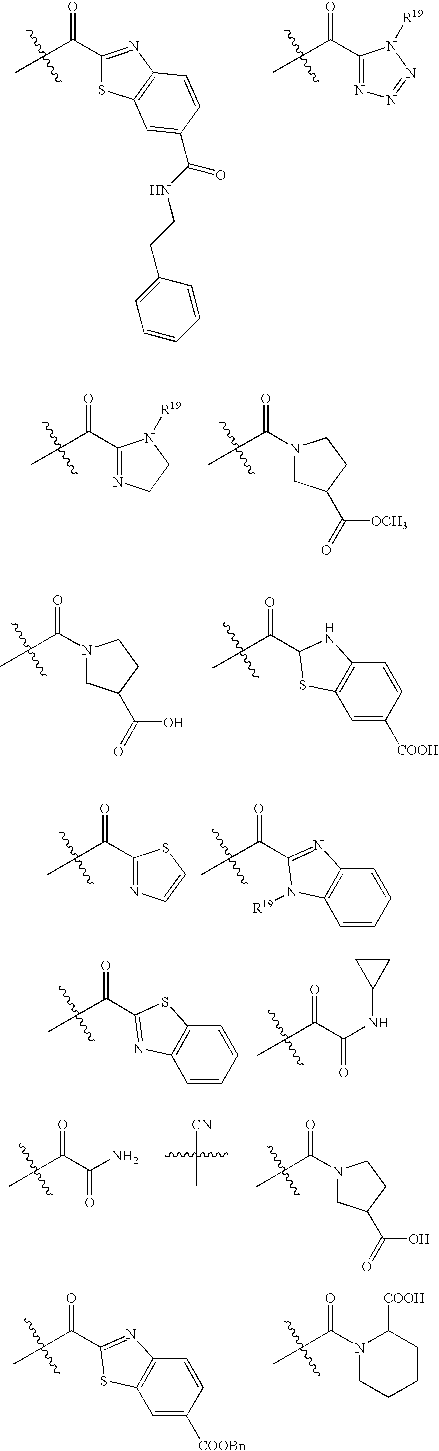 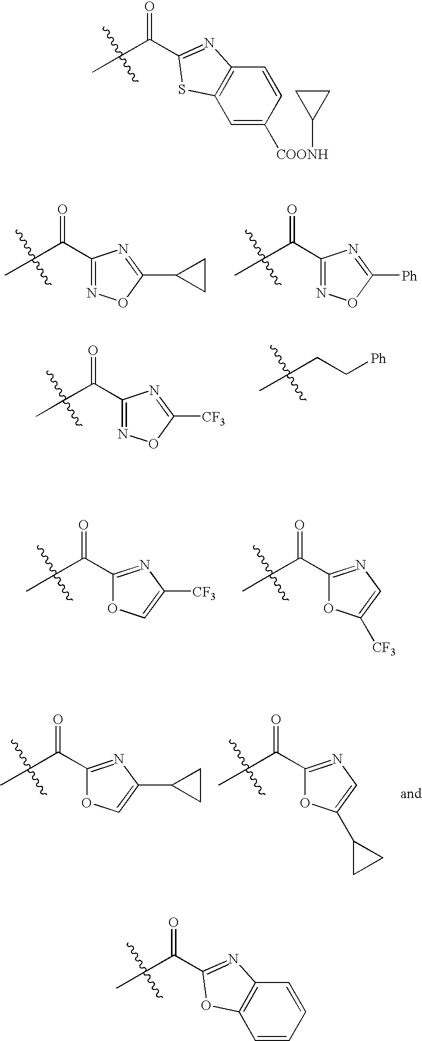
wherein R¹⁹ is selected from the group consisting of hydrogen, a halogen atom, aryl, trihalomethyl, and C₁₋₄-alkyl.

27. The compound of any one of the above claims, wherein R² is selected from the group consisting of 2,2-difluoroethyl, propyl, cyclobutyl-methyl and 2-cyclobutyl-ethyl.

28. The compound of claim 1, wherein R¹¹ is H and R¹² is C₃₋₆-cycloalkyl.

29. The compound of claim 1, wherein R¹² is cyclohexyl.

30. The compound of any one of the above claims, wherein V is C(O)—N(H)-t-butyl.

31. The compound of any one of the above claims, wherein V is C(O)—R²⁰, wherein R²⁰ is selected from the group consisting of C₃₋₆-cycloalkyl, phenyl, pyrazine, benzooxazole, 4,4-dimethyl-4,5-dihydro-oxazole, benzoimidazole, pyrimidine, benzothiazole 1,1-dioxide and quinazoline, each of which may be further independently substituted with a halogen atom, CF₃, C₁₋₄-alkyl or C₃₋₆-cycloalkyl.

32. The compound of any one of the above claims, wherein V is C(O)—R²⁰, wherein R²⁰ is selected from the group consisting of 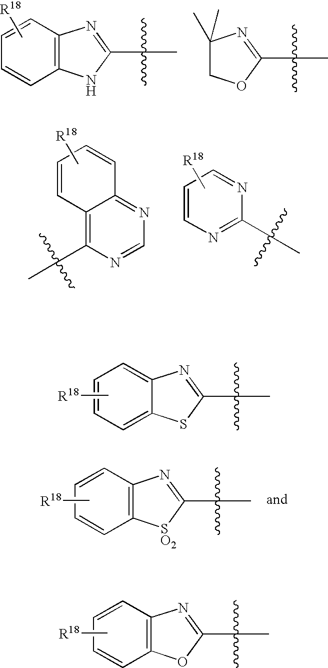
wherein R¹⁸ is selected from the group consisting of hydrogen, a halogen atom, aryl, trihalomethyl, and C₁₋₄-alkyl.

33. The compound of any one of the above claims, wherein V is C(O)—R²⁰, wherein R²⁰ is selected from the group consisting of 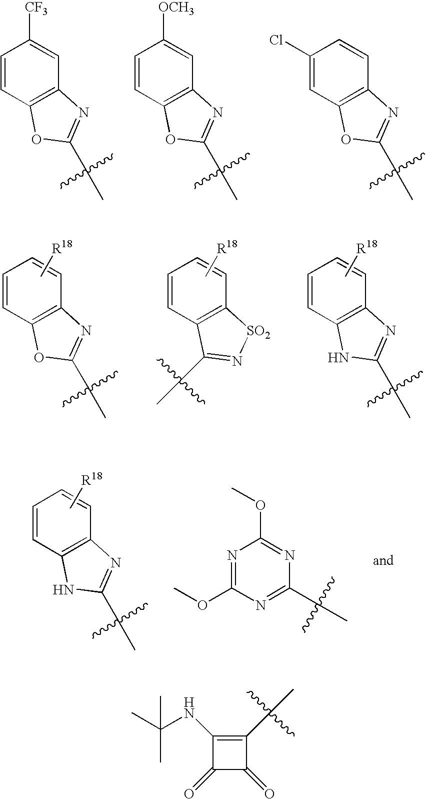
wherein R¹⁸ is selected from the group consisting of hydrogen, a halogen atom, aryl, trihalomethyl, and C₁₋₄-alkyl.

34. The compound of any one of the above claims, wherein V is selected from the group consisting of C₃₋₆-cycloalkyl, phenyl, pyrazine, benzooxazole, 4,4-dimethyl-4,5-dihydro-oxazole, benzoimidazole, pyrimidine, benzothiazole 1,1-dioxide and quinazoline, each of which may be further independently substituted with a halogen atom, CF₃, C₁₋₄-alkyl or C₃₋₆-cycloalkyl.

35. The compound of any one of the above claims, wherein V is selected from the group consisting of 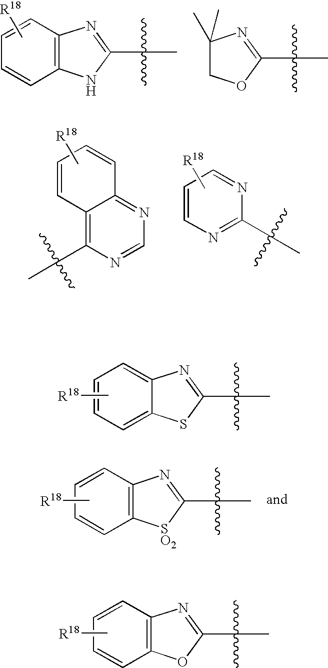
wherein R¹⁸ is selected from the group consisting of hydrogen, a halogen atom, aryl, trihalomethyl, and C₁₋₄-alkyl.

36. The compound of any one of the above claims wherein V is selected from the group consisting of 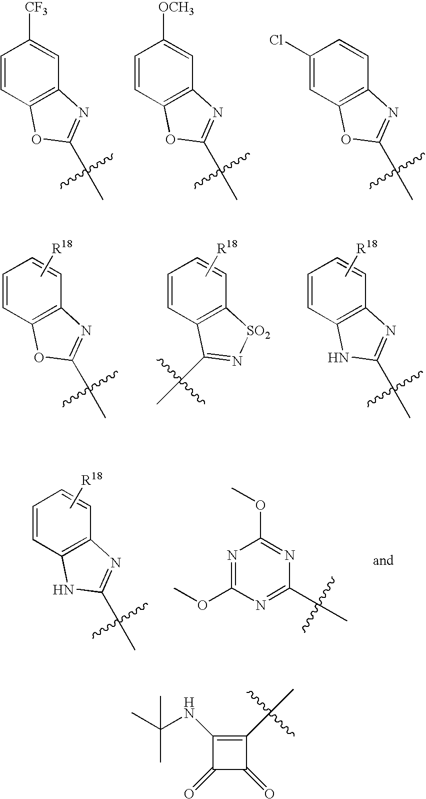
wherein R¹⁸ is selected from the group consisting of hydrogen, a halogen atom, aryl, trihalomethyl, and C₁₋₄-alkyl.

37. The compound of any one of the above claims, wherein W is C(O)—C(O)-amino.

38. The compound of claim 1, wherein R¹³ is H and V is selected from the group consisting of C═N(H)NH₂, C═N(CN)NH₂ and C(O)NH₂.

39. The compound of any one of the above claims, wherein W is C(O)N(H)S(O)₂R²⁴, wherein R²⁴ is selected from the group consisting of H, C₁₋₄-alkyl, (CH₂)₀₋₄-C₃₋₆-cycloalkyl, substituted or unsubstituted aryl and substituted or unsubstituted heterocycle, each of which may be independently substituted one or more times with a halogen atom or C₁₋₄-alkyl.

40. The compound of any one of the above claims, wherein W is COOH, R¹ is H, and R² is selected from the group consisting of propyl, 2,2-difluoroethyl and CH₂-cyclobutyl, or R¹ and R² form together a cyclopropyl group that may be further substituted with a vinyl group.

41. The compound of any one of the above claims, wherein R¹ and R² form a substituent of the following Formula:

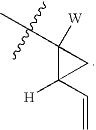

42. The compound of any one of the above claims, wherein W, R¹ and R² form a substituent of the following formula:

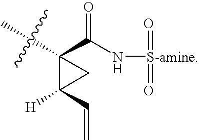

43. The compound of any one of the above claims, wherein W, R¹ and R² form a substituent of the following formula: 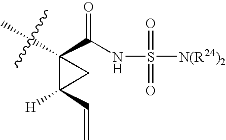
wherein each R²⁴ is independently selected from the group consisting of H, substituted or unsubstituted-C₁₋₄-alkyl, substituted or unsubstituted C₃₋₆-cycloalkylC₀₋₄alkyl, substituted or unsubstituted aryl and substituted or unsubstituted heterocycle.

44. The compound of any one of the above claims, wherein R²⁴ is selected from the group consisting of

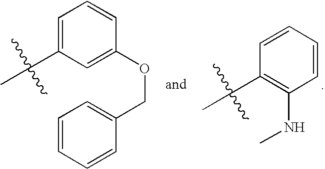

45. The compound of any one of the above claims, wherein W, R¹ and R² form a substituent selected from the group consisting of:

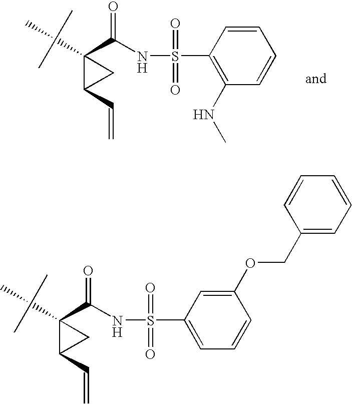

46. The compound of any one of the above claims, wherein V is selected from the group consisting of acyl, SO₂—R²⁴, C(O)N(R²⁴)₂, C(O)O(R²⁴)₂, and N(H)R²⁴, wherein each R²⁴ is hydrogen or is independently selected from the group consisting of amino, C₁₋₄-alkyl, mono- and di-C₁₋₄alkylamino, C₃₋₆-cycloalkylC₀₋₄alkyl, aryl, aryloxy and heterocycle, each of which may be independently substituted one or more times with a halogen atom or C₁₋₄-alkyl.

47. A method of treating an HCV-associated disorder comprising administering to a subject in need thereof a pharmaceutically acceptable amount of a compound of Formula I or II, such that the HCV-associated disorder is treated.

48. The method of claim 47, wherein the HCV-associated disorder is selected from the group consisting of HCV infection, liver cirrhosis, chronic liver disease, hepatocellular carcinoma, cryoglobulinaemia, non-Hodgkin's lymphoma, and a suppressed innate intracellular immune response.

49. A method of treating an HIV infection comprising administering to a subject in need thereof a pharmaceutically acceptable amount of a compound of Formula I or II.

50. A method of treating, inhibiting or preventing the activity of HCV in a subject in need thereof, comprising administering to the subject a pharmaceutically acceptable amount of a compound of Formula I or II.

51. A method of inhibiting the activity of a serine protease, comprising the step of contacting said serine protease with a compound according to claim 50.

52. The method of claim 50, wherein the activity of the NS2 protease is inhibited.

53. The method of claim 50, wherein the activity of the NS3 protease is inhibited.

54. The method of claim 50, wherein the activity of the NS3 helicase is inhibited.

55. The method of claim 50, wherein the activity of the NS5a protein is inhibited.

56. The method of claim 50, wherein the activity of the NS5b polymerase is inhibited.

57. The method of claim 50, wherein the interaction between the NS3 protease and NS4A cofactor is disrupted.

58. The method of claim 50, wherein the severing one or more of the NS4A-NS4B, NS4B-NS5A and NS5A-NS5B junctions of the HCV is prevented or altered.

59. The method of claim 50, wherein an HCV-associated disorder is treated in a subject in need thereof.

60. The method of claim 59, wherein the HCV-associated disorder is selected from the group consisting of HCV infection, liver cirrhosis, chronic liver disease, hepatocellular carcinoma, cryoglobulinaemia, non-Hodgkin's lymphoma, and a suppressed innate intracellular immune response.

61. A method of treating, inhibiting or preventing the activity of HCV in a subject in need thereof, comprising administering to the subject a pharmaceutically acceptable amount of a compound of Formula I or II, herein the compound interacts with any target in the HCV life cycle.

62. The method of claim 61, wherein the target is selected from the group consisting of NS2 protease, NS3 protease, NS3 helicase, NS5a protein and NS5b polymerase.

63. A method of decreasing the HCV RNA load in a subject in need thereof comprising administering to the subject a pharmaceutically acceptable amount of a compound of Formula I or II, such that the HCV RNA load in the subject is decreased.

64. A compound exhibiting HCV protease activity, wherein the compound is of the Formula I or II.

65. The compound of claim 64, wherein the compound is a HCV NS3-4A protease inhibitor.

66. A method of treating an HCV-associated disorder in a subject, comprising administering to a subject in need thereof a pharmaceutically acceptable amount of a compound of the Formula I or II, and a pharmaceutically acceptable carrier, such that the HCV-associated disorder is treated.

67. A method of treating an HCV-associated disorder comprising administering to a subject in need thereof a pharmaceutically effective amount of a compound of the formula I or II, in combination with a pharmaceutically effective amount of an additional HCV-modulating compound, such that the HCV-associated disorder is treated.

68. The method of claim 67, wherein the additional HCV-modulating compound is selected from the group consisting of Sch 503034 and VX-950.

69. The method of claim 67 wherein the additional HCV-modulating compound is interferon or derivatized interferon.

70. The method of claim 69, wherein the interferon is selected from the group consisting of interferon alpha 2B, pegylated interferon alpha, consensus interferon, interferon alpha 2A, lymphoblastoid interferon, and interferon tau; and said compound having anti-hepatitis C virus activity is selected from the group consisting of interleukin 2, interleukin 6, interleukin 12, a compound that enhances the development of a type 1 helper T cell response, double stranded RNA, double stranded RNA complexed with tobramycin, Imiquimod, ribavirin, an inosine 5′-monophosphate dehydrogenase inhibitor, amantadine, and rimantadine.

71. The method of claim 67 wherein the additional HCV-modulating compound is a cytochrome P450 monooxygenase inhibitor.

72. The method of claim 71, wherein the cytochrome P450 inhibitor is selected from the group consisting of ritonavir, ketoconazole, troleandomycin, 4-methyl pyrazole, cyclosporin, and clomethiazole.

73. The method of claim 66, wherein the HCV-associated disorder is selected from the group consisting of HCV infection, liver cirrhosis, chronic liver disease, hepatocellular carcinoma, cryoglobulinaemia, non-Hodgkin's lymphoma, and a suppressed innate intracellular immune response.

74. A method of inhibiting hepatitis C virus replication in a cell, comprising contacting said cell with a compound of Formula I or II.

75. A packaged HCV-associated disorder treatment, comprising an HCV-modulating compound of the Formula I or II, packaged with instructions for using an effective amount of the HCV-modulating compound to treat an HCV-associated disorder.

76. The treatment of claim 52, wherein the HCV-associated disorder is selected from the group consisting of HCV infection, liver cirrhosis, chronic liver disease, hepatocellular carcinoma, cryoglobulinaemia, non-Hodgkin's lymphoma, and a suppressed innate intracellular immune response.

77. A method of treating HCV infection, liver cirrhosis, chronic liver disease, hepatocellular carcinoma, cryoglobulinaemia, non-Hodgkin's lymphoma, and/or a suppressed innate intracellular immune response in subject in need thereof comprising administering to the subject a pharmaceutically acceptable amount of a compound of Formula I or II.

78. The method of claim 50, wherein the HCV is selected from any HCV genotype.

79. The method of claim 78, wherein the HCV is selected from HCV genotype 1, 2 and/or 3.

80. The method of claim 67, wherein the HCV-associated disorder is selected from the group consisting of HCV infection, liver cirrhosis, chronic liver disease, hepatocellular carcinoma, cryoglobulinaemia, non-Hodgkin's lymphoma, and a suppressed innate intracellular immune response.
